(19) United States
(12) Patent Application Publication
(10) Pub. No.: US 2026/0226101 A1
(43) Pub. Date: Aug. 6, 2026

(54) PEPTIDE WITH ANTI-INFLAMMATORY AND ANTI-MICROBIAL ACTIVITY

(71) Applicant: Ramakrishna Reddy ISANAKA, Hyderabad, Telangana (IN)

(72) Inventor: Ramakrishna Reddy ISANAKA, Hyderabad, Telangana (IN)

(21) Appl. No.: 19/147,138

(22) PCT Filed: Sep. 26, 2024
(86) PCT No.: PCT/IB2024/059383

§ 371 Date: Jul. 10, 2025

(30) Foreign Application Priority Data
Oct. 30, 2023 (IN) 202341073695

(51) Int. Cl. C07K 7/06; A61K 38/00; A61P 31/04; A61P 37/02; C07K 1/22

(52) U.S. Cl. CPC C07K 7/06; A61P 31/04; A61P 37/02; C07K 1/22; A61K 38/00

(57) ABSTRACT

The present invention relates to a novel synthetic peptide IS 111 with anti-inflammatory and anti-microbial activity. The present invention also discloses the synthetic peptide IS 111 as a potent inhibitor of sepsis.

### FIELD OF INVENTION

The present invention relates to a novel synthetic peptide IS 111 with anti-inflammatory and anti-microbial activity. The present invention also discloses the synthetic peptide IS 111 as a potent inhibitor of sepsis.

### BACKGROUND OF INVENTION

Sepsis is a severe clinical condition marked by disrupted inflammatory balance in response to infection(s), posing a life-threatening emergency. It most commonly affects vulnerable groups, such as the elderly, immunocompromised individuals, children, and infants. Recent data shows that sepsis was present in 32.5% of 200 COVID-19 hospitalizations, with 70.8% attributed solely to SARS-COV-2, 26.2% to both SARS-COV-2 and other infections, and 3.1% solely to bacterial infection. COVID-19 pneumonia is a significant contributor to sepsis in patients.

Sepsis can initiate in various body parts, including the lungs, urinary tract, skin, or gastrointestinal tract, and swiftly lead to tissue damage, organ failure, and death. According to the Centers for Disease Control and Prevention (CDC), one in three hospital deaths involve sepsis, and its incidence has risen over the past 30 years.

Infections, whether bacterial, fungal, or viral, can lead to sepsis. Gram-positive and Gram-negative bacteria, such as Staphylococcus aureus, Pseudomonas spp., and Escherichia coli, are common culprits. Escherichia coli infections are responsible for a significant portion of hospitalized patient infections and mortality worldwide.

Sepsis triggered by Gram-negative bacteria often involves lipopolysaccharide (LPS), which leads to systemic inflammatory responses. These responses result in the release of pro-inflammatory cytokines, causing a systemic inflammatory response syndrome characterized by symptoms like fever, tachycardia, and leukocytosis. This hyper-inflammatory response leads to organ failure, followed by a collapse of the host's defense mechanisms.

The frequency of sepsis is increasing, mortality rates are on the rise (50-60%), and hospitalization durations remain unchanged despite medical advancements. Developing anti-inflammatory antagonist-based therapies is crucial for viable antisepsis medication.

Currently, successful sepsis therapies rely on simple interventions: broad-spectrum antibiotics, early goal-directed therapies, and lung-protective ventilation strategies. These approaches face limited acceptance due to cost, bleeding risk, and lack of proven clinical benefit. Early and appropriate therapy remains essential for sepsis survival, as there is no specific treatment.

Sepsis remains a leading cause of death in intensive care units (ICU), and despite advances in diagnosis and pathogenesis understanding, no satisfactory therapy has emerged. The emergence of antimicrobial-resistant infections further underscores the need for effective treatments.

It is imperative to find an effective therapy for sepsis, considering its impact on medical procedures and the treatment of chronic diseases. Host immune defenses may also play a role in drug resistance, emphasizing the urgency of developing immunomodulatory therapies to enhance long-term patient outcomes. Efforts focus on identifying effective immunomodulating agents with anti-infective properties to improve sepsis outcomes.

Antimicrobial and immunomodulatory agents can be targeted to improve sepsis treatment. This peptide therapy offers an innovative approach to treating infectious diseases, combining antimicrobial and immunomodulatory properties. Synthetic peptides, frequently used in research and development, have seen over 150 peptide molecules enter clinical trials recently, indicating a shift in the pharmaceutical industry towards peptide-based drugs.

Antimicrobial peptides (AMPs) are newly discovered immune system components that act as effective agents against bacterial, yeast, and viral infections, potentially serving as alternatives to antibiotics. Designed antimicrobial peptides (dAMPs), inspired by natural peptides, show increased potency, specificity, and reduced toxicity. These peptides have demonstrated resistance to high solute levels and enhanced activity.

Immunomodulatory Synthetic Peptides, also known as Host Defense Peptides (HDPs), have gained recognition for their role in modulating innate immunity, often referred to as Innate Defense Regulator (IDR) peptides. These peptides not only combat infections but also influence immune-cell function, presenting a novel approach to infection treatment.

Antimicrobial peptides (AMPs) show promise as therapeutic agents against multi-drug resistant bacterial infections. They have advantages such as a broad activity range, minimal resistance development risk, and the ability to control host immune responses. However, limitations include low bioavailability and high cost, which are being addressed through various strategies, making AMPs a potential replacement for conventional antibiotics.

Immuno-Therapy: Targeting the Adaptive Immune System Research on human sepsis, particularly clinical trials, has been limited, with most studies being observational. Drugs targeting hyperinflammation must be administered early in the disease course, focusing on patients with elevated pro-inflammatory cytokine levels.

Sepsis patients often survive the hyper-inflammatory phase but remain hospitalized, at risk of immune compromise and secondary infections. Developing therapies to enhance host immune responses is crucial for reducing mortality in sepsis.

HDPs and their synthetic derivatives possess attributes that make them attractive for combating multi-drug resistant pathogens. Extensive research has explored their immunomodulatory properties, aiming to enhance their effectiveness.

Thus there is a need in the state of the art for peptides which have effective antimicrobial and immunomodulatory activities with a potential to treat sepsis.

Accordingly, the present invention provides a novel synthetic peptide IS 111, a seven-amino-acid synthetic β-sheet forming peptide (FAKKFAK), which exhibits dual antibacterial and immunomodulatory capabilities, representing an innovative approach to infectious disease treatment especially sepsis.

### BRIEF DESCRIPTION OF DRAWINGS

Other features, objects, and advantages of the present disclosure will become more apparent from the following detailed description of the non-limiting embodiments with reference to the accompanying drawings.

FIG. 1: Step wise procedure followed in in vitro activity.

FIG. 2: The picture representation of microdilution assay.

FIG. 3: Depiction of collection of peritoneal macrophages and the study parameters screened.

FIG. 4: Schematic representation of two most used sepsis models. (A) The injection of live bacteria E. coli (8739™) (Intraperitoneally). (B) The ceacal ligation and puncture (CLP) model by puncturing and ligating the cecum, faeces can reach the peritoneal cavity was established.

FIG. 5: Step wise procedure of the Cecum Ligation and Puncture (CLP) model. (a) Setup of the surgery table. (b) IP injection of anesthesia. (c) Confirmation of anesthesia by touch. (d) Shaving the surgery part of the mice. (e) Placing the mice on the surgery table and disinfecting the surgical part of the mice. (f) Skin incision. (g) Locating and exposing the cecum. (h&i) Ligated the cecum. (j) Puncturing the cecum with a 20-gauge needle. (k) Extruding the cecal matter/suspension. (l) Replacing the cecum back into peritoneal sac of the mice. (m&n) Skin closing suturing steps. (o) After the suturing. (p) Applying the betadine to the surgical part of the mice. (q & r) Placing the mice on the heating pad for the maintained of the body temperature and recovery of the anesthesia. (s) Post operated surgical mice after recovery from anesthesia.

FIG. 6: Study design and treatment regimen in CLP model.

FIG. 7: Confirmation of E. coli strain used in the study on UTI chrome plate.

FIG. 8: Study design and treatment regimen in E. coli-induced septic peritonitis model.

FIG. 9: This figure shows the collection of peritoneal lavages from the mice. [Meurer S K, 2016]

FIG. 10: HRMS chromatogram of peptide: IS 111

FIG. 11: HPLC chromatogram of peptide: IS 111.

FIG. 12: Morphological change in macrophage RAW 264.7 cells. (a-f) LPS-treated with test peptide IS 111 (3.12, 6.25, 12.5, 25, 50 and 100 μg/mL respectively, (g) Control/untreated, (h) LPS-(1 μg/mL) treated only, and (h) LPS-treated with dexamethasone (500 μg/mL).

FIG. 13(a): IL-1β secretion measured using ELISA.

FIG. 13(b): IL-6 secretion measured using ELISA.

FIG. 14(a): TNF-α secretion was measured using ELISA.

FIG. 14(b): IL-10 secretion was measured using ELISA.

FIGS. 15(a) to (d): Evaluation of the protein levels of P38 & ERK1/2 by western blot.

FIG. 16 shows effect of test peptide IS 111 on TNF-α mRNA expression was detected by RT-qPCR.

FIG. 17 (a, b) show effect of test peptide IS 111 on IL-6 & IL-1β mRNA expression was detected by RT-qPCR.

FIG. 18 shows effect of test peptide IS 111 on CCL2 mRNA expression was detected by RT-qPCR.

FIG. 19: Time-kill kinetics of test peptide IS 111 against S. aureus

FIG. 20: Time-kill kinetics of test peptide IS 111 against P. aeruginosa.

FIG. 21: Time-kill kinetics of test peptide IS 111 against E. coli.

FIG. 22: Time-kill kinetics of test peptide IS 111 against K. pneumoniae.

FIGS. 23(a) to (d): TNF-α (a), IL-6 (b) IL-1β (c) and IL-12p70 (d) cytokines level detection in vitro mice macrophages.

FIG. 24: IL-10 cytokines level detection in vitro mice macrophages.

FIG. 25(a): Molecular docking interactions of test peptide IS 111 with the receptor Map kinase. Active pocket of Map kinase in complex with 4-(4-fluorophenyl)-1-(4-piperidinyl)-5-(2-amino-4-pyrimidinyl)-imidazole. The color code representations for the picture as yellow: protein; magenta: co-crystal ligand. The amino acids represented in lines and co-ligand was in sticks.

FIG. 25(b): Molecular docking interactions of peptide IS 111 with the receptor Map kinase (1BL7).

FIG. 26: Molecular docking interactions of peptide IS 111 with the receptor TNF-alpha (4K8U).

FIG. 27: Molecular docking interactions of peptide IS 111 with the receptor VEGF1 (3HNG).

FIG. 28: Molecular docking interactions of peptide IS 111 with the receptor VGEF-2 (3VHE).

FIG. 29: Molecular docking interactions of peptide IS111 with the receptor VGEF-3 (4BSJ).

FIG. 30: This figure demonstrates that experimental mice show the signs of infection at 18 h after post E. coli ATCC 8739™ (5.0×10⁸ E. coli CFU/per mouse).

FIG. 31(a) shows that the short synthetic peptide IS 111 increases survival after 18 h of the treatment-after Sepsis Induction.

FIG. 31(b) shows the short synthetic peptide IS 111 increases survival after 5 days of the treatment-after Sepsis Induction.

FIGS. 32 (a) to (d): Detection of cytokines (IL-1B, IL-6, IL-12 & TNF-α) in the serum sample of animals after E. coli Infection, induction of sepsis and short synthetic peptide IS 111 treatment, after 18 h of polymicrobial sepsis.

FIG. 33: Detection of cytokines (IL-10) in the serum sample of animals after E. coli Infection, induction of sepsis and short synthetic peptide IS 111 treatment, after 18 h of polymicrobial sepsis.

FIG. 34 (a) and (b): Detection of cytokines (IL-6 & TNF-α) in the Peritoneal lavage fluid sample of animals after E. coli Infection, induction of sepsis and short synthetic peptide IS111 treatment, after 18 h of polymicrobial sepsis.

FIGS. 35 (a) to (c): Detection of Lymphocytes, WBC & Neutrophils counts in the serum sample of animals after E. coli infection, induction of sepsis and short synthetic peptide IS 111 treatment, after 18 h of polymicrobial sepsis.

FIG. 36: Photographs of representative sections of kidneys sections were prepared and stained with H&E. visualized at 200× magnification are shown. Data shown in mean±SEM from 3-4 mice of all groups of E. coli induced sepsis, treatments, and control animals-IV route −18 h.

FIG. 37: Photographs of representative sections of liver (a) and lungs (b) sections were prepared and stained with H&E. visualized at 200× magnification are shown. Data shown in mean±SEM from 3-4 mice of all groups of E. coli induced sepsis, treatments, and control animals-IV route-18 h.

FIG. 38: The short synthetic peptide IS 111 increases survival after 18 h of the treatment-after Sepsis Induction.

FIG. 39: The short synthetic peptide IS 111 increases survival after 7 days of the treatment—after Sepsis Induction.

FIGS. 40 (a) to (d): Detection of cytokines (IL-1β, IL-6, IL-12 & TNF-α) in the serum sample of animals after CLP surgery, induction of sepsis and short synthetic peptide IS 111 treatment, after 18 hrs of polymicrobial sepsis.

FIG. 41: Detection of cytokines (IL-10) in the serum sample of animals after CLP surgery, induction of sepsis and short synthetic peptide IS 111 treatment, after 18 hrs of polymicrobial sepsis.

FIG. 42: Photographs of representative sections of kidney sections were prepared and stained with H&E. visualized at 200× magnification are shown. Data shown in mean±SEM from 3-4 mice of all groups of CLP induced sepsis, treatments, and control animals-IV route-18 h.

FIG. 43: Photographs of representative sections of liver (a) and lungs (b) sections were prepared and stained with H&E. visualized at 200× magnification are shown. Data shown in mean±SEM from 3-4 mice of all groups of CLP induced sepsis, treatments, and control animals-IV route-18 h.

### SUMMARY OF INVENTION

The present invention provides a novel synthetic peptide IS 111 with anti-inflammatory and anti-microbial activity. The present invention also provides the synthetic peptide IS 111 as a potent inhibitor of sepsis.

The present invention investigates the anti-inflammatory and antimicrobial properties of a novel synthetic peptide IS 111 in both in vitro and in vivo settings using polymicrobial septic shock mouse models. To the best of the knowledge the present invention represents the first report demonstrating the potent inhibition of septic shock in mice by the novel peptide, IS 111.

In the studies conducted IS 111's anti-inflammatory effects on LPS-stimulated RAW 264.7 macrophage cells and murine macrophages were evaluated, along with its impact on MAPK signaling pathways to understand the inhibitory mechanism. Our methods included in vitro assays such as MTT assay, pro, and anti-inflammatory cytokine measurements, RT-qPCR, western blot, and in vivo studies involving E. coli-induced peritonitis and cecal ligation and puncture (CLP) models, accompanied by hematoxylin and eosin staining (H&E) to assess anti-inflammatory activity. The antibacterial activity through microdilution assays and time kill profiles against S. aureus, P. aeruginosa, E. coli, and K. pneumoniae was also examined.

The results indicated that LPS-induced inflammation led to increased pro-inflammatory cytokines TNF-α, IL-1β, and IL-6. IS 111 demonstrated a recovery from LPS-induced depression-like behaviour, associated with decreased pro-inflammatory cytokine production in both cell types without causing cytotoxicity at various dosages. Additionally, it suppressed the mRNA expression of pro-inflammatory mediators such as IL-1β, IL-6, TNF-α, and CCL2 (MCP1). IS 111 also showed a dose-dependent downregulation of IL-1β. Immunoblot analysis revealed that IS 111 induced anti-inflammatory signal transduction via the MAPK pathway, suggesting its potential as a potent anti-inflammatory agent.

The present invention also investigates the hypothesis that IS 111 administration could reduce organ failure and enhance survival in E. coli-induced peritonitis and CLP-induced sepsis mouse models. In the peritonitis model, IS 111 treatment at a dose of 1.2 mg/kg (IV) resulted in lower levels of IL-1β, IL-6, IL-12, and TNF-α in the serum and peritoneal fluid compared to the disease control group. Importantly, IS 111 administration significantly reduced mortality, serum IL-1β and TNF-α levels, macrophage infiltration into peritoneal fluid and lung tissues of CLP-mice. Peritoneal cells from IS 111-treated mice displayed characteristics of protective M2 macrophages, reducing excessive inflammation. IS 111 also inhibited disseminated intravascular coagulation, further preventing organ damage and improving survival. Therefore, IS 111 displayed a remarkable reduction in inflammation, along with antimicrobial activity, leading to improved septic mice survival.

Thus, IS 111 effectively suppressed inflammation in both in vitro and in vivo settings via the MAPK pathway and reduced proinflammatory cytokine production, neutrophil infiltration, and lung injury in septic mice. The findings disclosed herein suggest that IS 111 may serve as a novel therapeutic peptide for controlling sepsis, given its broad-spectrum antibacterial and anti-biofilm activity, coupled with immunomodulatory effects. It represents a promising therapy for inflammatory diseases associated with macrophage activation and presents a new potential target for sepsis treatment.

Accordingly the present invention discloses:
In vitro activity of peptide IS 111 for cytotoxicity and anti-inflammatory activity on murine macrophage RAW 264.7 cell lines stimulated with LPS.
In vitro activity of test peptide IS 111 for anti-microbial activity against Staphylococcus aureus, Pseudomonas aeruginosa, Escherichia coli and Klebsiella pneumoniae.
Confirmatory studies for in vitro activity of test peptide (IS 111) on BALB/C mice macrophages stimulated with LPS.
Acute toxicity of the test peptide IS 111.
Molecular docking studies of peptide IS 111 against about five targets, which have anti-inflammatory action.
Confirmatory in vivo studies to test the efficacy activity of test peptide IS 111 using E. coli induced peritonitis animal model & Cecal Ligation Puncture (CLP) Animal Model.

In an embodiment, the present invention provides a peptide with anti-inflammatory and anti-microbial activity, the peptide having an amino acid sequence of SEQ ID NO: 1 or variant thereof.

In another embodiment, the present invention provides a peptide with anti-inflammatory and anti-microbial activity, wherein the peptide has the chemical formula C₄₂H₆₆N₁₀O₈.

In one of the embodiments, the present invention provides that the peptide inhibits Interferon gamma (IFN-γ), thymus and activation regulated chemokine, Interleukin-8, thymic stromal lymphopoietin secretion, IL-β, IL-1β, IL-6, IL-10, IL-12, TNF-α, and CCL2 (MCP1).

In a further embodiment the present invention provides a formulation comprising a peptide of SEQ ID NO: 1 or a variant thereof or a peptide of chemical formula C₄₂H₆₆N₁₀O₈ and one or more suitable pharmaceutically acceptable excipients for treating, preventing, alleviating and/or ameliorating inflammatory and/or microbial diseases or one or more symptoms associated thereof.

In a still further embodiment the present invention provides that in the formulation said one or more suitable pharmaceutically acceptable excipients are selected from the group consisting of suitable carriers, starch, povidone, cellulose, lactose, magnesium stearate, microcrystalline cellulose, Poloxamer, Polysorbate 20, Sodium chloride, Sodium starch glycolate, anti-adherent, Calcium phosphate, stearic acid, colloidal silicon dioxide, colorants, sodium croscarmellose, diluents, crospovidone, glidant, mannitol, vehicles, disintegrant, swelling agent, antioxidant, buffer, bacteriostatic agent, emollient, emulsifier, plasticizer, penetration enhancer, preservative, cryoprotectant, neutralizer, fragrance additives, dispersants, surfactants, binders and lubricants.

In a yet another embodiment, the present invention provides that in the formulation the peptide is present in an amount of 0.01 μg/mL to 1000 μg/mL.

In another embodiment the present invention provides the peptide or the formulation of present invention for treatment, amelioration, alleviation and/or prevention of inflammatory and/or microbial disease and/or one or more symptoms associated thereof.

In one of the embodiments, the present invention provides a method of synthesizing peptide of SEQ ID No. 1 or a variant thereof comprising:
(a) preparing peptydil resin containing the peptide of SEQ ID No. 1 (IS111) or variant thereof with resin;
(b) performing cleavage of the peptydil resin cocktail obtained in step (b) to obtain crude peptide; and
(c) optionally purifying the crude peptide obtained in step (b) to obtain pure peptide.

In another embodiment the present invention provides that in the method of synthesizing peptide of SEQ ID NO: 1 or a variant thereof, step a) for preparing peptydil resin containing the peptide of SEQ ID NO: 1 (IS111) or variant thereof with resin comprises the following steps of:
(i) placing resin in a reaction vessel and swelling with at least one suitable solvent;
(ii) washing the resin with at least one suitable solvent;
(iii) deprotecting by adding piperdine in at least one suitable solvent to the resin followed by stirring and draining;
(iv) obtaining a solution of Fluorenylmethyloxycarbonyl tyrosine (Fmoc Tyr) and Hydroxybenzotriazole (HOBT) in at least one suitable solvent followed by adding N,N′-Diisopropylcarbodiimide (DIC) to the solution and adding solution to the resin;
(v) mixing and checking for free amino acid by Ninhydrine test;
(vi) repeating steps (i) to (v) with next amino acid to obtain peptydil resin cocktail.

In a further embodiment the present invention provides that in the method of synthesizing peptide of SEQ ID NO: 1 or a variant thereof, said suitable solvent in step a) is Dimethylformamide (DMF), acetonitrile, methanol, methyl ethyl ketone, l-butanol, t-butanol, tert-butyl methyl ether, trimethylamine, toluene or any combination thereof, preferably DMF.

In an embodiment the present invention provides a method of synthesizing peptide of SEQ ID NO: 1 or a variant thereof comprising:
(a) preparing peptydil resin containing the peptide of SEQ ID NO: 1 (IS111) or variant thereof with resin comprising the steps of
(i) placing resin in a reaction vessel of a synthesizer and swelling with Dimethylformamide (DMF);
(ii) washing the resin with DMF;
(iii) deprotecting by adding piperdine in DMF to the resin followed by stirring and draining;
(iv) obtaining a solution of Fluorenylmethyloxycarbonyl tyrosine (Fmoc Tyr) and Hydroxy benzotriazole (HOBT) in DMF N,N′-followed by adding Diisopropylcarbodiimide (DIC) to the solution and adding solution to the resin;
(v) mixing and checking for free amino acid by Ninhydrine test;
(vi) repeating steps (i) to (v) with next amino acid to obtain peptydil resin cocktail;
(b) performing cleavage of the peptydil resin cocktail obtained in step (b) to obtain crude peptide; and
(c) optionally purifying the crude peptide obtained in step (b) to obtain pure peptide.

In one of the embodiments, the present invention provides a method of treating a subject infected with inflammatory or microbial diseases for treating, preventing, alleviating and/or ameliorating said inflammatory or microbial diseases or one or more symptoms associated thereof comprising administering a therapeutically effective amount of a peptide of SEQ ID NO: 1 or variant thereof or peptide of chemical formula C₄₂H₆₆N₁₀O₈, or a formulation comprising a therapeutically effective amount of said peptide or variant thereof.

In a further embodiment, the present invention provides that in the method of treating a subject infected with inflammatory or microbial diseases wherein said formulation is capable of inhibiting one or more of Interferon gamma (IFN-γ), thymus and activation regulated chemokine, Interleukin-8, thymic stromal lymphopoietin secretion, IL-β, IL-1β, IL-6, IL-10, IL-12, TNF-α, and CCL2 (MCP1).

In a further embodiment the present invention provides that in the method of treating a subject said administration is in dosage form selected from oral, sub-cutaneous, topical, intraperitoneal, intra-venous or combination thereof.

In a further embodiment the present invention provides that in the method of treating a subject said therapeutically effective amount of the peptide or variant thereof in said formulation is 0.01 μg/ml to 1000 μg/ml.

In a still further embodiment the present invention provides that in the method of treating a subject said administration is at a dosage of about 0.01 mg/kg to 1000 mg/kg.

In one of the embodiments the present invention provides use of a peptide or a formulation of the invention for preparation of a medicament for treating, preventing, alleviating or ameliorating severity of inflammatory or microbial diseases or one or more symptoms associated thereof in an individual.

### DESCRIPTION OF INVENTION

For a better understanding of the present disclosure, various aspects of the present disclosure will be described in more detail with reference to the accompanying drawings. It should be understood that the detailed description is merely illustrative of the exemplary embodiments of the present disclosure and is not intended to limit the scope of the present disclosure in any way. Throughout the specification, the same reference numerals refer to the same elements. The expression “and/or” includes any and all combinations of one or more of the associated listed items.

The present invention provides a novel synthetic peptide IS 111 with anti-inflammatory and anti-microbial activity. The present invention also provides the synthetic peptide IS 111 as a potent inhibitor of sepsis.

The present invention provides a pharmaceutical formulation comprising a peptide of SEQ ID NO: 1 or a variant thereof or a peptide of chemical formula C₄₂H₆₆N₁₀O₈ and one or more suitable pharmaceutically acceptable excipients for treating, preventing, alleviating and/or ameliorating inflammatory and/or microbial diseases or one or more symptoms associated thereof.

The formulation of present invention comprises one or more suitable pharmaceutically acceptable excipients wherein said one or more suitable pharmaceutically acceptable excipients are selected from the group consisting of suitable carriers, starch, povidone, cellulose, lactose, magnesium stearate, microcrystalline cellulose, Poloxamer, Polysorbate 20, Sodium chloride, Sodium starch glycolate, anti-adherent, Calcium phosphate, stearic acid, colloidal silicon dioxide, colorants, sodium croscarmellose, diluents, crospovidone, glidant, mannitol, vehicles, disintegrant, swelling agent, antioxidant, buffer, bacteriostatic agent, emollient, emulsifier, plasticizer, penetration enhancer, preservative, cryoprotectant, neutralizer, fragrance additives, dispersants, surfactants, binders and lubricants. Further, the pharmaceutical formulation may include a pharmaceutical carrier. The pharmaceutical carrier may be any carrier as long as it is a non-toxic material suitable for delivering peptide or variant thereof to patients. For example, distilled water, alcohol, fats, waxes and inactive solids may be included as a carrier. Pharmaceutically acceptable adjuvants (buffering agents, dispersants) may also be included in the pharmaceutical formulations. In these formulations, the concentration of the peptide or variant thereof may vary greatly.

The pharmaceutically acceptable excipients in the formulations of the invention include, but are not limited to, fillers, diluents, disintegrants, binders, lubricants, antioxidants, surfactants, pH modifiers, anti-sticking, coating polymers, stabilizers and the like, and any combinations thereof.

Suitable grades of controlled release polymers may also be included in the formulation. Said polymers may include, but are not limited to, non-ionic soluble cellulose either, such as hydroxypropyl methylcellulose, hydroxypropyl methylcellulose acetyl succinate, hydroxypropyl cellulose, hydroxyethyl cellulose (HEC e.g., Natrosol); insoluble cellulose polymers such as ethyl cellulose etc., non-ionic homopolymers of ethylene oxide, such as poly(ethylene oxide) with a molecular weight range of 100,000 to 8000,000; water soluble natural gums of polysaccharides of natural origin, such as xanthan gum, alginate, and locust bean gum; water swellable, but insoluble, high molecular weight homopolymers and copolymers of acrylic acid chemically cross-linked with poly-alkenyl alcohols with varying degree of cross-linking or particle size (Carbopol 71G NF, 971P, 934P); polyvinyl acetate and povidone mixture (e.g. Kollidon S R); Cross-linked high amylose starch or Ionic methacrylate copolymers (Eudragit L30D) alone or in combination were selected.

Diluents that can be included in pharmaceutical formulations are, but are not limited to, microcrystalline cellulose (MCC), silicified MCC microfine cellulose, lactose, lactose monohydrate, starch, pregelatinized starch, sugar, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and any mixture thereof.

Binders that can be used in the pharmaceutical formulations to help hold tablets together after compression are, but are not limited to, acacia, guar gum, alginic acid, carbomers, dextrin, maltodextrin, methylcelluloses, ethyl celluloses, hydroxyethyl celluloses, hydroxypropyl celluloses, hydroxypropyl methylcelluloses, carboxymethylcellulose sodium, liquid glucose, magnesium aluminum silicate, polymethacrylates, polyvinylpyrrolidones, copovidone, gelatin, starches, and any mixtures thereof.

Disintegrants that can be used in the pharmaceutical formulations are, but are not limited to, croscarmellose Sodium, methylcelluloses, microcrystalline celluloses, carboxymethyl cellulose calcium, carboxymethyl cellulose sodium, crospovidones, povidones, guar gum, magnesium aluminum silicate, colloidal silicon dioxide (AEROSIL™), polacrilin potassium, starch, pregelatinized starch, sodium starch glycolate, sodium alginate, and any mixtures thereof.

Specifically, the pharmaceutical formulations may contain a formulation material for altering, maintaining, or conserving the pH, osmolarity, viscosity, transparency, color, isotonicity, odor, sterility, stability, dissolution or release rate, adsorption, or permeability of the composition. Examples of the suitable formulating material may include amino acids (e.g., glycine, glutamine, asparagine, arginine or lysine), anti-microorganism agents, anti-oxidants (e.g., ascorbic acid, sodium sulfite or sodium bisulfite), buffering agents (e.g., borate, bicarbonates, Tris-HCl, citrate, phosphate or other organic acids), bulking agents (e.g., mannitol or glycine), chelating agents (e.g., ethyelenediaminetetraacetic acid (EDTA)), complexing agents (e.g., caffeine, polyvinylpyrrolidione, β-cyclodextrin or hydroxypropyl-β-cyclodextrin), fillers, monosaccharides, disaccharides and other carbohydrates (e.g., glucose, mannose or dextrin), proteins (e.g., serum albumin, gelatin or immunoglobulin), coloring agents, flavoring agents, diluents, emulsifiers, hydrophilic polymers (e.g., polyvinylpyrrolidione), low molecular weight polypeptides, salt-forming counterions (e.g., sodium), preservatives (e.g., benzalkonium chloride, benzoic acid, salicylic acid, thimerosal, phenethyl alcohol, methylparaben, propylparaben, chlorhexidine, sorbic acid or hydrogen peroxide), solvents (e.g., glycerin, propylene glycol or polyethylene glycol), sugar alcohols (e.g., mannitol or sorbitol), suspending agents, surfactants or humectants (e.g., pluronics; PEG; sorbitan ester; polysorbate, e.g., polysorbate 20 or polysorbate 80; triton; tromethamine; lecithin; cholesterol or tyloxapol), stability improvers (e.g., sucrose or sorbitol), growth improvers (e.g., alkali metal halides, preferably, sodium chloride or potassium chloride; or mannitol, sorbitol), delivery vehicles, diluents, excipients and/or pharmaceutical adjuvants, but are not limited thereto.

The pharmaceutical formulations may include additional pharmaceutically acceptable excipients, including any one or more of glidants, lubricants, surfactants such as sodium lauryl sulphate, and other commonly used excipients. This list, and the foregoing listings of representative specific excipients, is not intended to be exhaustive, as those skilled in the art will be aware of other substances that can be used.

The formulations of the present invention may comprise the peptide or variant thereof in an amount of 0.01 μg/mL to 1000 μg/mL. The formulations of the present invention may comprise the peptide or variant thereof preferably in an amount of 0.1 μg/mL to 100 μg/mL.

The present invention also provides a method of treating a subject infected with inflammatory or microbial diseases comprising administering a therapeutically effective amount of a formulation comprising a therapeutically effective amount of peptide of SEQ ID No. 1 or variant thereof, or peptide of chemical formula C₄₂H₆₆N₁₀O₈.

This method includes, in particular, administering an effective amount of the peptide or variant thereof of the present invention to a subject in need thereof.

The pharmaceutical formulations of the present invention may be administered via any route. The formulations of the present invention may be provided to a subject directly (e.g., topically, by administering into tissue areas by injection, transplantation, or by topical administration) or systemically (e.g., by oral- or parenteral administration) via any appropriate means. When the composition of the present invention is parenterally provided via intravenous-, subcutaneous-, ophthalmic-, intraperitoneal-, intramuscular-, oral-, rectal-, intraorbital-, intracerebral-, intracranial-, intraspinal-, intraventricular-, intrathecal-, intracistenal-, intracapsular-, intranasal-, or aerosol administration, the formulation is preferably aqueous or may include a portion of a physiologically applicable body liquid suspension or solution. Accordingly, the carrier or vehicle may be added to the formulation and be delivered to a patient since it is physiologically applicable. Therefore, a physiologically-appropriate saline solution may generally be included as a carrier like a body fluid for formulations.

Further, the administration frequency may vary depending on the pharmacokinetic parameters of the peptide or variant thereof in the formulations to be used. Typically, physicians would administer the formulation until an administration dose to achieve a desired effect is reached. Accordingly, the formulation may be administered as a unit dose, at least two doses with time intervals (may or may not contain the same amount of a target peptide or variant thereof) or administered by a continuous injection via a transplantation device or catheter. The precision of addition of an appropriate administration dose may be routinely performed by those skilled in the art, and corresponds to the scope of work being routinely performed by them.

Additionally, the preferable unit dose of the peptide or variant thereof in a subject in need thereof may be in a range from 0.001 mg/kg to 1000 mg/kg of body weight, and more preferably from 0.1 mg/kg to 500 mg/kg of body weight. Although this is the optimal amount, the unit dose may vary depending on the disease to be treated or the presence/absence of adverse effects. Nevertheless, the optimal administration dose may be determined by performing a conventional experiment. The administration of the peptide or variant thereof may be performed by a periodic bolus injection, an external reservoir (e.g., an intravenous bag), or a continuous intravenous-, subcutaneous-, or intraperitoneal administration from the internal source (e.g., a bio-erodible implant).

In addition, the peptide or variant thereof of the present invention may be administered to a subject recipient along with other biologically active molecules. The optimal combination of the peptide or variant thereof and other molecule(s), dosage forms, and optimal doses may be determined by a conventional experiment well known in the art.

The simplified peptide sequence allows for rapid, cost-effective production and quicker clinical application. The novel synthetic peptide IS 111 (SEQ ID NO.: 1-Phe-Ala-Lys-Lys-Phe-Ala-Lys of the present invention), a seven-amino-acid synthetic β-sheet peptide with broad-spectrum antibacterial and immunomodulatory properties, demonstrates great potential.

The present invention provides a method of synthesising peptide of SEQ ID No. 1 or a variant thereof comprising:
(a) preparing peptydil resin containing the peptide of SEQ ID No. 1 (IS111) or variant thereof with resin;
(b) performing cleavage of the peptydil resin cocktail obtained in step (b) to obtain crude peptide; and
(c) optionally purifying the crude peptide obtained in step (b) to obtain pure peptide.

In the method of present invention the step a) for preparing peptydil resin containing the peptide of SEQ ID No. 1 (IS111) or variant thereof with resin comprises the following steps of:
(i) placing resin in a reaction vessel and swelling with at least one suitable solvent;
(ii) washing the resin with at least one suitable solvent;
(iii) deprotecting by adding piperdine in at least one suitable solvent to the resin followed by stirring and draining;
(iv) obtaining a solution of Fluorenylmethyloxycarbonyl tyrosine (Fmoc Tyr) and Hydroxybenzotriazole (HOBT) in at least one suitable solvent followed by adding N,N′-Diisopropylcarbodiimide (DIC) to the solution and adding solution to the resin;
(v) mixing and checking for free amino acid by Ninhydrine test;
(vi) repeating steps (i) to (v) with next amino acid to obtain peptydil resin cocktail.

In the method of synthesising peptide provided by the present invention, said suitable solvent in step a) is Dimethylformamide (DMF), acetonitrile, methanol, methyl ethyl ketone, l-butanol, t-butanol, tert-butyl methyl ether, trimethylamine, toluene or the likes or any combination thereof.

In the method of synthesizing peptide provided by the present invention, said suitable solvent in step a) is preferably DMF.

The present invention provides a method of synthesising peptide of SEQ ID NO: 1 or a variant thereof preferably comprising the following steps:
(a) preparing peptydil resin containing the peptide of SEQ ID NO: 1 (IS111) or variant thereof with resin comprising the steps of
(i) placing resin in a reaction vessel of a synthesizer and swelling with Dimethylformamide (DMF);
(ii) washing the resin with DMF;
(iii) deprotecting by adding piperdine in DMF to the resin followed by stirring and draining;
(iv) obtaining a solution of Fluorenylmethyloxycarbonyl tyrosine (Fmoc Tyr) and Hydroxybenzotriazole (HOBT) in DMF followed by adding N,N′-Diisopropylcarbodiimide (DIC) to the solution and adding solution to the resin;
(v) mixing and checking for free amino acid by Ninhydrine test;
(vi) repeating steps (i) to (v) with next amino acid to obtain peptydil resin cocktail;
(b) performing cleavage of the peptydil resin cocktail obtained in step (b) to obtain crude peptide; and
(c) optionally purifying the crude peptide obtained in step (b) to obtain pure peptide.

Due to these properties, including its antimicrobial and anti-inflammatory activity, peptide IS 111 was initially studied for its broad-spectrum antibacterial effects against both gram-positive and gram-negative strains. Subsequently, its immunomodulatory properties were discovered. The peptide was further evaluated for its effectiveness in treating sepsis induced by extra-intestinal pathogenic E. coli in animals and confirmed using the reliable Cecal Ligation and Puncture (CLP) sepsis model.

Table 1, below lists the reagents and antibodies used for conducting the studies of the present invention. Table 2, below lists the instruments used for conducting the studies of the present invention.

TABLE 1
List of the reagents and antibodies used in studies

| Material | Catalogue | Company |
|---|---|---|
| Test Peptide- IS 111 | — | Issar Pharmaceuticals Pvt. Ltd |
| Macrophage Cell line | TIB- 71 ™ | American Type Culture |
| Raw 264.7 cells | | Collection, USA. |
| Staphylococcus aureus subsp. | ATCC ® 6538 ™ | American Type Culture |
| aureus | | Collection, USA. |
| Pseudomonas aeruginosa | ATCC ® 9027 ™ | American Type Culture |
| | | Collection, USA. |
| Escherichia coli | ATCC ® 8739 ™ | American Type Culture |
| | | Collection, USA. |
| Klebsiella pneumoniae subsp. | ATCC ® 700603 ™ | American Type Culture |
| Pneumoniae | | Collection, USA. |
| Lipopolysaccharide (LPS, | LPS25 | Sigma-Aldrich (St. Louis, MO, |
| Escherichia coli 0111: B4) | | USA). |
| Dulbecco's modified Eagle's | 30-2002 ™ | American Type Culture |
| medium (DMEM) | | Collection, USA. |
| Fetal Bovine Serum (FBS) | Gibco, 10082147 | Gibco BRL Life Technologies |
| | | (Grand Island, NY, USA) |
| Antibiotics (streptomycin/ | 15140122 | Gibco BRL Life Technologies |
| penicillin) | | (Grand Island, NY, USA) |
| Ciprofloxacin | Honeywell Fluka | Fluka. |
| | R1678-100MG | |
| Methyl Thiazolyl Tetrazolium | M2128-100MG | Sigma (St Louis, MO, USA). |
| (MTT) | | |
| IL-6 | DY406-05 &M6000B | R&D Systems (USA). |
| TNF-α | DY410-05&MTA00B | R&D Systems (USA). |
| IL-1β | DY401-05 & MLB00C | R&D Systems (USA). |
| IL-10 | DY417-05 | R&D Systems (USA). |
| IL-12 | DY419 & D1200 | R&D Systems (USA). |
| Mouse procalcitonin ELISA | NBP2- 812212 | Novus. |
| kit (Calorimetric) | | |
| VEGF | SMMV00 | R&D Systems (USA) |
| cDNA synthesis kit | AB1453B | Thermo Fischer Scientific |
| Sybr mix | RR820A | TAKARA (USA) |
| Trizol | 9109 | TAKARA (USA) |
| PVDF membrane | 1620177 | Biorad (USA) |
| TRIS Base | 0210313301 | MP Biomedicals |
| RIPA | 20-188 | Sigma Aldrich (Germany) |
| Triton X-100 | T8787 | Sigma chemicals Co., MO, USA |
| BCA detection kit | 23225/BCA1 | Thermo Scientific, Rockford, IL, |
| | | USA/Sigma Aldrich (Germany) |
| PMSF | 10837091001 | Sigma Aldrich (Germany) |
| Protease Inhibitor | P8340-1ML | Sigma Aldrich (Germany) |
| Protein ladder | PG-PMT2922 | Invitrogen (USA) |
| BSA | MB083-100G | Himedia Laboratories |
| Non-fat dry milk powder | Sc-2325 | Santacruz |
| RNA iso Plus | 9108/9109 | Takara |
| Enhanced chemiluminescence | 32109 | Thermo Fisher Scientific, |
| (ECL) Western blotting | | Waltham, MA, USA |
| detection reagent | | |
| TLR4 | A5258 | ABclonal |
| TNF-α | ab205587 | ABclonal |
| IL-10 | A2171 | ABclonal |
| IL-6 | A1115 | ABclonal |
| p38 | 8690 | CST |
| phosphorylated (p)-38 | 4511 | CST |
| ERK | 4695 | CST |
| p-ERK | 4370 | CST |
| GAPDH | 5174 | CST |
| Dimethylsulfoxide (DMSO) | 673439 | Sigma Chemical Co. (St. Louis, |
| | | MO, USA). |
| Phosphate buffered saline | 10010023 | Gibco BRL Life Technologies |
| (PBS) | | (Grand Island, NY, USA) |
| Luria Bertani Agar/Miller | M1151 | (MHA, Mast Diagnostics, U.K.) |
| Mueller Hinton broth and agar | 70192 M | HiMedia Laboratories, Mumbai, |
| plates | | Maharashtra, India |

TABLE 2
List of instruments used in the study.

| Instrument Name | Make |
|---|---|
| 96-Well Fast Thermal | Veriti Applied Biosystems |
| Cycler | |
| Automated peptide synthesizer | Applied Biosystems model 433A |
| Centrifuge Sorvall ST 16R | Thermo Fisher Scientific |
| Chemiluminescence detection | Fusion SL, Vilber Lourmat |
| system | |
| HPLC | Shimadzu, Japan. |
| HRMS | Thermo scientific ™ Q orbitrap |
| | exploris ™ 120 mass |
| | spectrometer and couple |
| | vanquish ™ 3000UHPLC. |
| Mice rectal probe | Physitemp, Clifton, NJ |
| Microscope | Olympus |
| Multi detection Microplate Reader | Biotek |
| Nanodrop (UV/Vis Spectrometer) | Thermo Fisher Scientific |
| Real Time PCR System | Step One Plus Applied Biosystems |
| Spectrophotometer | Multiskan GO Thermo Scientific |
| | 51119300, Thermo Fisher |
| | Scientific, USA |
| Weighing Balance | Sartorius |
| Western-blot apparatus | Bio-rad |

IS 111 i.e. the novel peptide was supplied by the Applicant's company-Issar Pharmaceuticals and 25 mg/ml was used as stock solution were prepared in Normal saline and working concentrations were made by serially dilution in CAMHB solution, 25 μg/ml as highest concentration and 0.195 μg/ml as the lowest concentration. Dilutions are 0.195, 0.391, 0.78, 1.56, 3.13, 6.25, 12.5 & 25 μg/ml for Anti-microbial assays.
Test compound Name: IS111
Test compound Code: ISSAR 02 (generated at test facility):
Physical Appearance: White colored Amorphous Powder
Chemical nature: Synthetic peptide-API
Stability: 12 months
Storage Conditions: 2-8° C. (For Short term)
   −20° C. (For long term)
   To be protected from light and moisture
Molecular Weight: 839.04 g/mole
Purity: 99.2%
Solubility: Soluble in water
8-10-week-old male BALB/C mice were used for the studies in the present invention. Animals were procured from Jeeva Life sciences Limited, Hyderabad (CPCSEA Registered animal supplier).
Identification: Temporary body marking: During acclimatization, the mice are temporarily identified by the body marking with crystal violet solution/indelible ink mark as bands.
   Permanent body marking: During treatment, the mice are permanently identified by cage cards and body marking with saturated turmeric solution in 70% alcohol.
Randomization: Animals are selected and grouped by manual randomization.
Acclimatization: BALB/C mice were acclimatized to the study area conditions for 1 week before they were used in the study.

### Test Conditions

### Animal Housing and Environment

Conditions: Standard Laboratory Conditions. All experimental animals used in this study were under a protocol approved by the Institutional animal ethics committee (IAEC)
IAEC Approval No.: BITS-Hyd/IAEC/2020/41 & Appendix-I
Temperature: 22±3° C.
Humidity: 30-70%.
Photoperiod: Light cycle of 12 hours light and 12 hours dark.
Accommodation: One mouse per sex per cage for all groups will be housed in sterilized suspended polypropylene cages (internal size: L 355×W 225×H 175 mm), with stainless steel top grill having facilities for pelleted food and drinking water in polypropylene bottle with stainless steel sipper tube. During the experiment, all animals will be housed in a single experimental room.
Diet: Animals were provided standard rodent feed ad libitum.
Water: Clean water obtained from Reverse Osmosis system ad libitum during acclimatization and study period.

Grouping of animals was carried out two days prior to the initiation of the treatment. It was done by manual randomization method by body weight stratification and distribution as follows: the mice procured for the study were weighed and grouped into body weight ranges (Males: 20 to 28 g). These body weight stratified mice were distributed randomly to all the study groups to attain group mean body weights not varying by more than ±20%. Animals with extreme body weights were not included in the studies.

The test peptides IS 111 were synthesized manually in a stepwise manner at a 0.1-10 mmol scale on a using N-Fmoc (N-fluorenylmethyloxycarbonyl) solid phase peptide synthesis strategy (multichannel peptide synthesizer) and where the peptides of >10-200 mmol were synthesized by automated peptide synthesizer. Peptides were purified by preparative reversed-phase HPLC (Agilent 1200) using with a C-18 coated, 10-micron bead column (Phenomenex Jupiter C18, 10 μm, 300 Å, 250×10 mm) using a gradient of 0.1% TFA in water (Mobile phase A) and 100% acetonitrile (ACN) (Mobile phase B) and characterized by RP-HPLC chromatography and MALDI-TOF mass spectrometry at In-house. The molecular weights were confirmed by mass spectrometry HRMS-LCMS. The purity was about 93-95% as determined by analytical HPLC. The peptides used in all biological assays were higher than 90% purity.

All commercially available chemicals and solvents of synthesis grade were used without further purification. The qualitative ninhydrin test was performed for each step to confirm completion of coupling. Purification was done by using reverse phase chromatography. The purity of the final compounds was examined by HPLC, (on Phenomenex C8 (150*4.6 mm, 5 μm, 100 Å) double end-capped RP-HPLC column)) and was greater than 95%.

### In Vitro Anti-Inflammatory Activity of Peptide IS 111 Against LPS-Induced Inflammation in RAW 264.7 Cells:

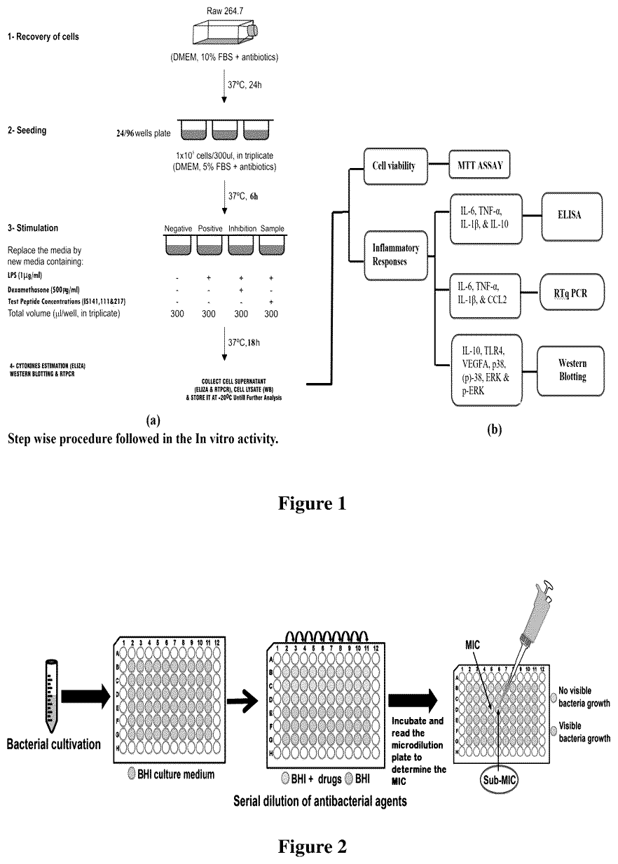

The methods for testing the in vitro anti-inflammatory activity of peptide IS 111 against LPS-induced inflammation in RAW 264.7 cells include cytotoxicity and anti-inflammatory assays which were conducted as shown in FIG. 1.

### Cell Line Culture:

The murine macrophage RAW264.7 cells were recovered from the stock and cultured in Dulbecco's modified Eagle's medium (DMEM) supplemented with 10% low-endotoxin heat-inactivated Fetal Bovine Serum (FBS), 2 μM glutamine, 100 U/ml penicillin, and 100 μg/ml streptomycin and cultured in a humidified atmosphere at 37° C. under 5% CO₂ until the cells were confluent around 70-80%. The growth medium was frequently replaced after 2-3 days. The cells were then washed and harvested using trypsin-EDTA. Cell culture has been done according to the methods describe by [Novilla et al. 2017; Rashad A. Al-Salahi et al., 2013 & Amit Kumar et al., 2021].

### Preparation of Synthetic Test Peptides:

For in vitro experiments, the synthetic test peptides IS 111 was dissolved in and diluted in 0.9% sterile normal saline (NS) for further concentrations used in the assay.

### Study Design:

The anti-inflammatory activities of IS 111 were evaluated in lipopolysaccharide (LPS)-stimulated RAW 264.7 cells. For establishment of in vitro model, lipopolysaccharide (LPS, 1 μg/mL) was used to treat the cells for 18 h. RAW 264.7 Cells were seeded at a density of 1×10⁵ cells/ml, either into 24/6-well plates for cytokines measurement by ELISA, western blot, and RT-PCR, or in 96-well plates for the cell viability assay. The experimental design is summarized in FIG. 2.

### Cytotoxicity Test—MTT Assay:

Cell viability of synthetic peptides on the growth of RAW264.7 cells was measured using the methyl thiazolyl tetrazolium (MTT) assay after 48 h. Briefly, exponentially growing macrophages RAW264.7 cells were mechanically scraped, plated at a density of 7×10³ cells in 100 μl were seeded into each well of a 96-well plate then incubated for 12 h at 37° C. under 5% CO2, and then the cells are treated with test peptides: IS 111 (1.5625, 3.12, 6.25, 12.5, 25, 50 and 100 μg/mL) or dexamethasone (500 μg/mL) cells were incubated at 37° C. in the presence of 5% CO2 for 42 h. After incubation, the morphology of cells was examined under a microscope. Following incubation, MTT was added to the cells (20 μL at 5 mg/mL MTT dissolved in 1×PBS) and incubated for 4 hours in the presence of 5% CO2 at 37° C. Formazan crystals were dissolved by the addition of 100 μL of 100% DMSO per well. Plates were then gently swirled for 5 min at room temperature to dissolve the precipitate. Absorbance was monitored at 575 nm using a microplate reader. Maximum cytotoxicity (100%) was determined by cells incubated with 1% Triton X-100; PBS was used as a negative control. Cells without treatment were considered as control. The average absorbance for each sample group was used to evaluate cell viability.

Optical density (O.D.) was calculated as the difference between the absorbance at the reference wavelength and that at the test wavelength. The relative cell viability was determined by the amount of MTT converted to the insoluble formazan salt. The data were expressed as the mean percentage of viable cells when compared with untreated cells (control). The half maximal growth inhibitory concentration (IC50) value was calculated from the line equation of the dose-dependent curve of each compound. The results were compared with the cytotoxic activity of dexamethasone, a known anti-inflammatory drug. The concentrations of test peptides that showed significant (p<0.001) cell viability was further selected for in vitro assays. Percentages of cell viability and LC50 of cells were calculated: Percent cell viability was calculated as (O.D. of drug-treated sample/control O.D.)×100. The data were expressed as percent cell viability compared with control (dimethyl sulfoxide/NS). The experiment was carried out in four samples of each concentration.

The percentages of cell viability and LC50 of cells were calculated by using the formula below.

$$\% cell viability = \frac{Absorbance sample}{Absorbance control} \times 100 ,$$

Where Absorbance control is the DMSO 0.1%-treated cell absorbance, and Absorbance sample is the test sample-treated cell absorbance. LC50 values were derived from dose-response curves, and data were expressed as the mean of three independent experiments. Statistical analyses involved Student's t-test and one-way ANOVA.

### Anti-Inflammatory Test in LPS-Stimulated RAW264.7 Cells:

The methods of in vitro anti-inflammatory assay have been done according to the methods describe by [Laksmitawati et al., 2017; Rusmana et al., 2015; Sandhiutami et al., 2017; Widowati et al., 2018].

### Cell Culture and LPS Stimulation:

Cells were sub-cultured by scraping when plates reached 70-80% confluence with a 1:5 ratio in fresh medium. RAW 264.7 cells were plated at density of 1×10⁵ cells and allowed for attachment. The growth medium was then replaced with fresh medium without FBS and incubated for 6 h then cells were pre-treated with different concentrations of synthetic peptides: IS 111 (3.12, 6.25, 12.5, 25, 50 and 100 μg/mL) or dexamethasone (500 μg/mL) as a positive control for 1 h followed by stimulation of LPS (1 μg/mL) and then further incubated for 18 h and were allowed to adhere. After 18 h of incubation, plates were centrifuged for 6 min at 400×g and the supernatants and cell lysates were collected and kept frozen at −80° C. until used for further analysis.

At the end of the treatment period, cells were harvested in RNA isoplus reagent for subsequent cellular RNA extraction, for RTqPCR. The pro & anti-inflammatory cytokine production in supernatants was estimated by using ELISA, RTqPCR and western blotting in cell lysates. The experiment was carried out in triplicate. LPS and Dexamethasone were used as the controls.

For analysis, samples of cells or culture supernatant were obtained after 18 h of treatment.

The following treatments were applied for the anti-inflammatory test:
(1) The negative control: RAW 264.7 cells without being induced by lipopolysaccharide; no drug treatment.
(2) The positive control: RAW264.7 cells that were induced by 10 μg/mL of lipopolysaccharide (LPS group).
(3) The mixture of Synthetic peptides: IS 111 (3.12, 6.25, 12.5, 25, 50 and 100 μg/ml), LPS (1 μg/mL) and RAW 264.7 cells; and
(4) The standard positive control: The mixture of dexamethasone (500 μg/mL), LPS (1 μg/mL), and RAW 264.7 cells.

### Measurement of Pro & Anti-Inflammatory Cytokine Markers:

The levels of IL-6, TNF-α, IL-1β, and IL-10 in the supernatants of RAW 264.7 cell cultures were determined using an ELISA kit, according to the manufacturer's instructions (R&D Systems).

TABLE 3
Cytokines dilutions used at the time of ELISA

| Cytokines | Dilution |
|---|---|
| TNF-α | 1:300 |
| IL-6 | 1:25 |
| IL-1β | No dilution |
| IL-10 | 1:20 |

### Western Blot Analysis:

As previously described, RAW264.7 cells were cultured and treated with LPS and synthetic peptides. After 18 h, cells were washed with PBS and lysed by incubating with RIPA lysis buffer cocktail (RIPA Lysis buffer, PMSF (1 mM), Protease inhibitor (1 μg/mL), phosphatase inhibitor (1 mM)) at −80° C. for 30 minutes. Post incubation, cell lysate was collected by scraping cells and centrifuged at 12000 rpm for 15 min.

The protein concentration was determined using the Bicinchoninic acid kit method with bovine serum albumin as the standard (provided in the kit), according to the manufacturer's instructions. Protein bands were electrophoretically transferred to a polyvinylidene difluoride (PVDF) membrane via semi-dry transfer blotting, Membranes were blocked with 5% BSA in PBST (1% Tween 20 in PBS) at 20-24° C. for 1 h, then incubated with primary antibodies against IL-10, TLR 4, VEGF, P38 p-p38, ERK and p-ERK, the dilutions are listed in table 3., at 4° C. overnight. GAPDH was used as a protein-loading control. The membrane was washed 3 times (10 min. each) with Tris-buffered saline/Tween 20 (TBST) and then treated with horseradish peroxidase (HRP)-conjugated secondary antibody (1:10000) for 1 h. The membrane was washed again with TBST. The membrane-binding antibodies were visualized later with the ECL detection reagent and images were acquired in a Fusion SL Imaging System. Band density was analyzed by densitometry using Image J software.

TABLE 4
Antibody dilutions used for western blotting

| Primary antibody | Dilution |
|---|---|
| p38 | 1:1000 |
| phosphorylated (p)-38 | 1:1000 |
| ERK | 1:1000 |
| p-ERK | 1:1000 |
| GAPDH | 1:1000 |

The Buffer compositions are mentioned as below.

TABLE 5
Resolving gel composition

| Resolving gel(10 ml/gel) | 8% | | 10% | | 12% | | 15% | |
|---|---|---|---|---|---|---|---|---|
| H2O | 4.6 | ml | 3.8 | ml | 3.2 | ml | 1.2 | ml |
| 30% Acrylamide | 2.6 | ml | 3.4 | ml | 4 | ml | 6 | ml |
| 1.5M Tris (pH 8.8) | 2.6 | ml | 2.6 | ml | 2.6 | ml | 2.6 | ml |
| 10% SDS | 0.1 | ml | 0.1 | ml | 0.1 | ml | 0.1 | ml |
| 10% APS | 0.1 | ml | 0.1 | ml | 0.1 | ml | 0.1 | ml |
| TEMED | 10 | μl | 10 | μl | 10 | μl | 10 | μl |

TABLE 6
Stacking gel composition

| Stacking gel(5 ml/gel) | 4% | |
|---|---|---|
| H2O | 2.975 | ml |
| 30% Acrylamide | 0.67 | ml |
| 0.5M Tris (pH 6.8) | 1.25 | ml |
| 10% SDS | 0.05 | ml |
| 10% APS | 0.05 | ml |
| TEMED | 5 | μl |

Running Buffer Composition (x)—1 Litre
1. Tris base—3.02 μm
2. Glycine—14.4 μm
3. SDS—1 μm,
4. Water—1000 ml

### Transfer Buffer Composition (1×)—1 Litre

1. Tris base—3.02 μm
2. Glycine—14.4 μm
3. Methanol—200 ml,
4. Water—800 ml

TABLE 7
Loading dye/laemmli buffer composition

| Laemmli buffer | 2x dye(10 ml) | |
|---|---|---|
| 10% SDS | 4 | ml |
| 1M Tris | 1.2 | ml |
| Bromophenol blue | 2 | mg |
| β-mercapto ethanol | 1 | ml |
| Glycerol | 2 | ml |
| Water | 1.8 | ml |

### ECL Buffer

1. 1M Tris buffer (pH7.2)—10 ml
2. 30% H2O2—5-10 μl (Depends on how many times it exposes to light)
3. P-Coumaric acid (90 mM)—22 μl
4. Luminol (250 mM)—50 μl
   Mix all the reagents just before membrane incubation.

### 1×PBS Preparation: 1 Litre

1. 8 g of NaCl.
2. 0.2 g of KCl.
3. 1.44 g of Na2HPO4.
4. 0.24 g of KH2PO4.
5. Adjust the pH to 7.4 with HCl
   Add distilled water to a total volume of 1 litre.

### Reverse Transcription—Polymerase Chain Reaction

Total RNA Extraction, Purification, and cDNA Synthesis:

Total cellular RNA was isolated using the RNA isoplus reagent (Takara Bioscience, India) following the manufacturer's instructions. RNA was quantified spectrophotometrically by absorption measurements at 260 and 280 nm using the nano drop system.

### CDNA Synthesis Steps:

As per table 8, RNA and the reaction mixture were mixed in PCR a tube and incubated in a thermal cycler for 5 min at 65° C. in and then cooled immediately on ice. Further, in the same tube, the step 2 reaction mixture was added and further incubated in the thermal cycler as mentioned below.

TABLE 8
cDNA synthesis procedure Step-1

| S. no | Reagent | Volume for 1 reaction from the kit | |
|---|---|---|---|
| 1. | Random6mers(50 μM) | 1 | μL |
| 2. | dNTPMixture (10 mMeach) | 1 | μL |
| 3. | RNase Free dH2O | 3 | μL |
| 4. | Template RNA | 5 | μL |
| | Total | 10 | μL |

TABLE 9
cDNA synthesis procedure Step-2

| | | Volume for 1 reaction | |
|---|---|---|---|
| S. No | Reagent | from the kit | |
| 1. | Template RNA Primer Mixture | 10 | μL |
| | (From step 1) | | |
| 2. | 5X PrimeScript Buffer | 4 | μL |
| 3. | RNase Inhibitor (40 U/μl) | 0.5 | μL |
| 4. | PrimeScript RTase (200 U/μl) | 1 | μL |
| 5. | Water | 4.5 | μL |
| | Total | 20 | μL |

Mixed gently.
And incubated the reaction mixture using the following conditions.
   30° C.—10 min (required when using Random 6 mers)
   42° C. (50° C.)—30-60 min
   Inactivated the enzyme by incubating at 95° C. for 5 min and then cooled on ice.
   After cDNA synthesis, samples were stored at −80° C. till used for RT-qPCR.

### RT-qPCR:

Primers were designed using the Primer3 online tool. GAPDH was used as a normalizing control. The sequence of primers used is provided in below table 10. Real-Time PCR was performed according to the manufacturer's protocol using Takara biosystem's real-time PCR mix (Takara, Bio-India) with ROX as a passive reference dye using applied Biosystem's step-one Real-Time PCR system. The following PCR program was used for all real-time PCR-based experiments: initial denaturation at 95° C. for 2 minutes, followed by 40 cycles of denaturation at 95° C. for 15 seconds, annealing at 60° C. for 30 seconds, extension at 60° C. for 15 seconds.

Real-time PCR was performed with step one plus Real-Time PCR system (Thermo Scientific). PCR reactions were performed in 20-μL reactions with SYBR Green Real time PCR Master Mix (Toybo, Osaka, Japan).

The primer sequences used for PCR amplification are as follows:

TABLE 10
Primer Sequences used in the RT-qPCR

| Gene | Primer sequence | |
|---|---|---|
| Mouse-TNF-α | Forward | 5′-AAGCCTGTAGCCCACGTCGTA-3′(SEQ ID No.: 2) |
| | Reverse | 5′-GGCACCACTAGTTGGTTGTCTTTG-3′ (SEQ ID No.: 3) |
| Mouse-IL-1β | Forward | 5′-CCAAGCAACGACAAAATACC-3′ (SEQ ID No.: 4) |
| | Reverse | 5′-GTTGAAGACAAACCGTTTTTCC-3′ (SEQ ID No.: 5) |
| Mouse-IL-6 | Forward | 5′-GAACAACGATGATGCACTTGC-3′ (SEQ ID No.: 6) |
| | Reverse | 5′-CTTCATGTACTCCAGGTAGCTATGGT-3′ (SEQ ID No.: 7) |
| Mouse-CCL2 | Forward | 5′-GCTACAAGAGGATCACCAGCAG-3′ (SEQ ID No.: 8) |
| | Reverse | 5′-GTCTGGACCCATTCCTTCTTGG-3′ (SEQ ID No.: 9) |
| GAPDH | Forward | 5′-AGG CCG GTG CTG AGT ATG TC-3′ (SEQ ID No.: 10) |
| | Reverse | 5′-TGC CTG CTT CAC CAC CTT CT-3′ (SEQ ID No.: 11) |

### Gene Expression Analysis:

For quantification of gene expression, CT values of each gene were normalized to GAPDH and calibrated to the appropriate control sample using the SYBR Green-based comparative CT method (2-ΔΔCt). Fold change was calculated using the formula 2-ΔΔct.

### Statistical Analysis:

All the experiments were performed in three replications, and data were expressed as the mean±standard deviation (SD). Differences in mean values between groups were analyzed by a one-way analysis of variance followed by Dunnett's-test for comparisons between two independent groups. P<0.001, P<0.01& P<0.05 was considered to indicate a statistically significant difference and denoted as **, *** & ** respectively. Graphs were plotted using Graph-pad prism-8.2/9.0.

### Confirmatory In Vitro Studies:

### In Vitro Evaluation of Anti-Microbial Activity of Test Peptide IS 111:

The aim of this study was to determine the spectrum of antimicrobial activity of synthetic test peptide IS 111. The anti-microbial activity was assessed by broth microdilution time-kill viability assays and inhibition of bio film assays.

### Microbial Strains:

The following were used as test bacteria: Staphylococcus aureus subsp. aureus (ATCCR 6538™), Pseudomonas aeruginosa (ATCCR 9027™), Escherichia coli (ATCC® 8739™) and Klebsiella pneumoniae subsp. Pneumoniae (ATCC® 700603™).

### Growth Media:

The bacteria cultures were maintained in cryotubes at −80° C. in 15% glycerol. A single colony of bacteria were refreshed in Mueller Hinton broth and incubated for 12 h at 37° C. and inoculated on Mueller Hinton agar plates for purity check. Overnight-cultured bacterial cells were transferred to MH medium and cultured to the exponential phase (optical density at 600 nm OD600=1.0). The culture was centrifuged and resuspended in sterile phosphate buffered saline (PBS) and adjusted to a final amount of 1×10⁵ CFU ml⁻¹ by using the equation CFU ml⁻¹_OD600 1-2.5×10⁸ [Evelien Gerits et al., 2016]. The number of colony-forming units was determined, and the bacterial cultures were adjusted to 5×10⁴ cfu/mL. Plates were prepared under aseptic conditions.

### Peptide Details:

The peptide IS 111 was synthesized by Issar Pharmaceuticals Pvt. Ltd., (India) and the lyophilized peptide of 10 mg vials was supplied and stored at −20° C. until usage. For in vitro and in vivo experiments, IS 111 was dissolved in 0.9% normal saline while preparing stock and working solutions. The Peptide purity used in biologic assays was higher than 90-95%.

### Antibiotics Used in the Study:

The following antibiotic was used in this study: Ciprofloxacin (Fluka): 10 mg/ml and working concentrations 0.5 & 1 μg/ml were prepared in Normal saline.

### Study Design

### Test Formulation:

25 mg/ml stock solution of test compound was prepared in Normal saline solution.

### Test Dilutions:

0.195, 0.391, 0.78, 1.56, 3.13, 6.25, 12.5 & 25 μg/ml.

### Standard Antibiotic:

Ciprofloxacin as a Positive control

### Positive Control Formulation:

10 mg/ml stock solution of positive control was prepared in Normal saline solution.

### Positive Control Concentrations Used for Treatment:

0.5& 1.0 μg/ml.

### Test Organisms:

Staphylococcus aureus,
Pseudomonas aeruginosa
Escherichia coli and
Klebsiella pneumoniae

### Treatment Regimen:

24 hrs

### Duration of Incubation:

24 hrs

### End Point:

Determination of Minimum Inhibitory Concentration (MIC) of the test peptide IS 111.

### Determination of the Minimal Inhibitory Concentration (MIC): Microdilution Assay:

Minimum inhibitory concentration (MIC) of peptide and antibiotic was evaluated using the broth microdilution technique in BHI with an initial inoculum of 1-2.5×10⁸ cells in non-treated polystyrene microtiter plates (Corning, USA) as described by [Wiegand et al., & Banfi et al., 2003]. The MICs were interpreted as the lowest concentration of peptide or antibiotic that completely inhibited the visible growth of bacteria after 24 h of incubation at 37° C. FIG. 2 shows the representation of microdilution assay.

IS111 MICs against Escherichia coli ATCC 8739 and Staphylococcus aureus ATCC 6538, Pseudomonas aeruginosa ATCC 9027 and Klebsiella pneumoniae ATCC 700603 were determined using a standardized broth microdilution assay according to CLSI reference methods for bacteria [Clinical Laboratory Standards Institute 2006]. All the bacterial cultures growth, propagation, preservation was done by following the ATCC bacterial culture guidebook and Stock solution (10 mg/ml) and Dilution series (0.195, 0.391, 0.78, 1.56, 3.13, 6.25, 12.5 & 25 μg/ml) of the test compound IS 111 were prepared in 0.9% Nacl solution in micro-test tubes from where they were transferred to 96-well microtiter plates. Then, 100 μL of each dilution were transferred into a 96-well microplate in 3×8 columns. Bacterial suspension (100 μL) was inoculated in each well with 1-2.5×10⁸ CFU/mL of all test organisms to obtain final concentrations of 2.5×10⁸ CFU/mL and a final volume of 200 μL per well. The inoculum (positive control) and culture medium (negative control) were put into the first column of the microplate, and the ciproflaxin antibiotic control ranging from 0.5 & 1.0 μg/ml in the final column.

Finally, the microplate was incubated with a sterile film cover for 24 h at 37° C. Bacterial growth was detected by optical density using ELISA reader, Spectra Max M5 multi-detection reader and checked the OD600. The results were expressed in micrograms per milliliters. The MIC was defined as the lowest concentration of antibacterial agent that resulted in the complete inhibition of visible growth [49]. The bacterial growth was indicated by the presence of turbidity meaning. Three independent experiments were performed, each with three biological replicates. Ciproflaxin was used as a standard antibacterial agent. Finally, microplate was incubated with a sterile film cover for 18-24 h at 37° C. Subsequently, 20 μL of 4% resazurin bacterial growth indicator was added to wells, which were then incubated for 30 min at 37° C. The lowest concentration of essential oil that visually showed no growth was determined as MIC. The MIC was determined as the lowest tested concentration that leads to complete inhibition (100%) in comparison to the negative-control group. Antibacterial activity was expressed as the concentration of extract inhibiting bacterial growth by 50% (IC50) [Mounyr Balouiri et al., 2016].

### Time-Kill Assay:

The survival of bacteria, time kill efficacy assay of IS 111 was further evaluated for activity against bacterial strain mentioned above according to CLSI reference method, with slightly modification. MICs were determined by broth microdilution assay as described above. The test compound IS 111 was incorporated into 4.9 ml Mueller Hinton Broth (MHB) at concentrations of 0.5×MIC, 1×MIC and 5×MIC for each bacterial species. Test tubes of MHB without test compound IS 111 were used as vehicle controls. Overnight cultures of the bacterial strains at cell densities of approximately 1×10⁸ CFU/ml were used to inoculate both test and control tubes. The standard tube dilution method was opted to evaluate the time-kill efficacy of bacteria. Bacterial inoculum (1 mL) was diluted by adding 9 mL sterile saline solution and serially diluted up to 10⁻³. On testing the bacterial dilution (10⁻³), bacterial colonies were observed in a discrete form which was easily countable. One mL of 10⁻³ bacterial cultures were then incubated with an equal amount of IS 111 in a shaker at 37° C. for 1, 2, 3, 4, 5, 12 and 24 h. 100 μL incubated suspension was transferred on the agar plates and spread through the spreader. Colony counts were performed after 24 h incubation at 37° C. Plates with 10-300 colonies were used for these counts, and the kill rate was determined by plotting log 10 viable counts (CFU/ml) against time. Bactericidal activity was defined as a ≥3 log 10 decreases in CFU/ml of the initial microbial population, while bacteriostatic activity was defined was defined as a <3 log 10 decrease in CFU/ml. The assay was performed in triplicate [Olufunmiso O Olajuyigbe et al., 2015; Sutthiwan Thammawat et al., 2017]

### In Vitro Anti-Inflammatory Activity of Test Peptide IS 111 in BALB/C Mice Peritoneal Macrophages:

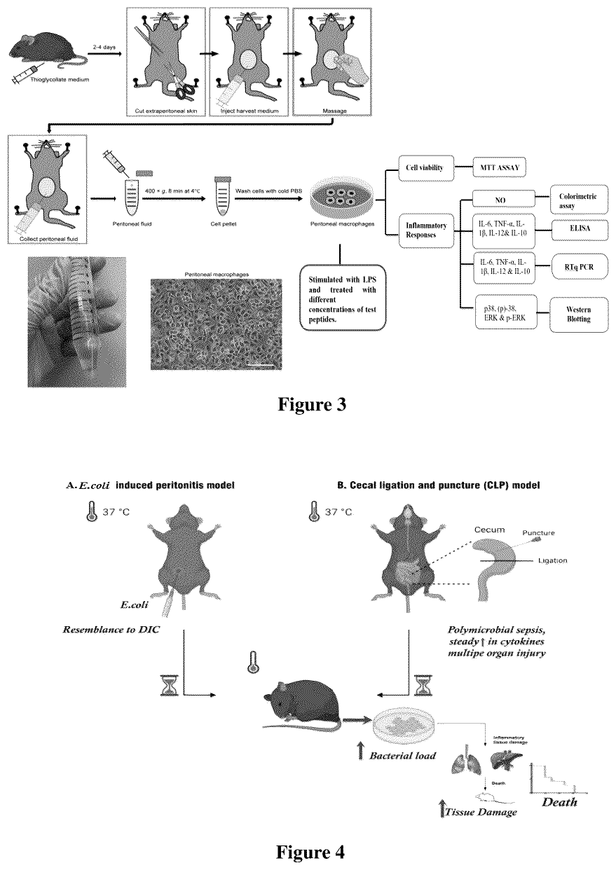

BALB/c mice (6-8 wk old) of either sex was injected IP with 1 ml of 4% sterile thioglycollate broth in PBS. After 4 d, mice were sacrificed, and peritoneal macrophages were harvested as described below [Meurer S K, 2016] and this model is well known as “Thioglycollate-induced peritonitis model”. FIG. 3 shows the collection of peritoneal macrophages and the study parameters screened.

### Preparation of Peritoneal Macrophages:

After 4 days, mice were then euthanized, and the peritoneal macrophages were harvested by lavaging the peritoneal cavity with 5-6 mL of harvest medium (EDTA 5 mM+PBS). Isolated peritoneal exudate cells were washed twice with RPMI and centrifuged at 1000 rpm/400×g for 8-10 min at 4° C. Finally, the cell suspension was dispensed in complete RPMI-1640 with 10% fetal bovine serum and allowed to adhere to the bottom of the 6 cm culture plate at 37° C. for 4 hr in 5% CO₂. The plates were then washed with warm PBS to remove non-adherent cells. The attached cells were considered as macrophages with 90% purity [Meurer S K, 2016].

### Cell Culture and Treatments:

Macrophages were seeded either in 96-well plates at a density of 0.2×10⁶ or in 24-well plates at a density of 0.8×10⁶ and were treated with different concentration (3.125, 6.25 & 12.5 μg/mL) of test peptide IS 111 for 1 h and then activated with 1 mg/ml of bacterial LPS (Sigma-Aldrich) and were cultured Dulbecco's modified Eagle's medium (DMEM) supplemented with 10% fetal bovine serum (FBS), 100 U/ml penicillin, and 0.1 mg/ml streptomycin and maintained under sterile conditions of 37° C., 5% CO2, and 85% humidity. After 24 h, culture supernatants were collected for estimation of cytokines by ELIZA and RTqPCR, whereas cells were harvested for western blotting. The procedure of ELISA, RTqPCR and other conditions are same as in the section. [Min Jee Kim et al., 2014].

### Statistical Analysis:

The experimental data were performed in triplicate and expressed as mean±SEM. The proportions of cells surviving at different time points were calculated, as the mean and standard deviation. Proportions of surviving cells were compared using Student's 2-tailed t-test, assuming unequal variance and differences were considered significant where P <0.005, P<0.05 and denoted as *** & ** and ^{###} & ^{##} when compared with standard drug and vehicle control groups respectively. The data were analyzed following Analysis of Variance (ANOVA) using graph pad prism 8.2/9.0.

### Acute Toxicity of Peptide IS 111

While developing novel therapeutic proteins/peptides, preventing immunogenicity, and establishing the acute toxicity profile are important issues to consider and the animal models are used to study immunogenicity prediction and acute toxicity of therapeutic proteins. The acute toxicity studies were conducted in compliance with the guideline of Organization for Economic Cooperation and Development (OECD) and schedule Y Guidelines for acute oral toxicity and previous work [OECD 401, 402, 423 & 425; SCHEDULE Y, DCGI-CDSCO document] with some modifications.

### Experimental Procedure of Acute Toxicity Study

TABLE 11
Test system

| | |
|---|---|
| IAEC Protocol No. | BITS-Hyd/IAEC/2020/17 |
| Animal Model used | Mice |
| Strain | BALB/C mice |
| Gender | Males/Females |
| Age & Body Weight | 6-8 weeks & 25-30 g |
| Test Doses | 0.6, 2.4, & 4.8 mg/kg |
| Test Route | Intravenous (IV) |
| Volumes | 0.1 ml |

Once after acclimation period, a total of forty mice for IV route of administration, forty mice (half male and half female)/route were randomized based on bodyweight divided into four groups (10 in each group/per route, in each group 5 per sex) per strain via IV route of administration; prior to dosing initiation (table 12). On the day of dosing (designated as Day 0 of the study), the mice were 6 weeks old, and their body weights ranged from 20 to 25 g. The dosing volume was 0.1 ml per mice and the actual volume of peptide IS 111 solution administered was calculated based on body weight measurements obtained on the day of administration and was administered IV in increasing doses of the synthetic test peptide IS 111 0.6, 2.4, & 4.8 mg/kg; (administration once). The routes of administration were the same proposed for use in humans [Food and Drug Administration, 1988]. Prior to dosing the animals were deprived of food for 12 hours but had free access to drinking water. Food was provided 3 hours after dose administration and was available ad libitum thereafter.

TABLE 12
Allocation of BALB/C for IV administration

| | Dose | IV |
|---|---|---|
| | (mg/kg | No. of Animals |
| Test groups | b. wt) | (M/F) |
| Control | 0 | 5/5 |
| IS 111 -Low dose (LD). | 0.6 mg/kg | 5/5 |
| IS 111-Medium dose (MD). | 2.4 mg/kg | 5/5 |
| IS 111-High dose (HD). | 4.8 mg/kg | 5/5 |

The dose calculations are mentioned later in the specification along with Table 36.

After a single dose administration, mortality and clinical signs associated with toxicity were observed and recorded daily for consecutive two weeks; body weight changes were measured before and after administration on the 14th day. The animals were observed frequently during the first 4-6-hour period following dose administration and once daily, thereafter, for 7 days, during which animals were evaluated for mortality, clinical signs of toxicity, and behavior. Each animal was weighed on Day 0 (prior to dosing) and on Day 7 during the observation period. At the end of the observation period, on day 14, after being weighed, animals were fasted for 12 h (free access to water) and anesthetized with ketamine (80-100 mg/kg BW) and xylazine in a dose (5-10 mg/kg) intraperitoneally (for 20 g mice ketamine of 0.2 ml and xylazine of 0.1 ml) as in combination the anesthetized with 0.2 ml via, IP. Blood samples were collected from orbit into nonheparinized Eppendorf tubes for the determination of serum biochemical parameters. Then animals were euthanized with co₂ Inhalation/isoflurane, and a complete necropsy was performed. The main organs of the thoracic and abdominal cavities and the brain were examined macroscopically for gross abnormalities. The number of animals killed for each of the doses was noted and the LD50 calculated by the Up and Down method, which is one of the most used to reduce the number of animals used [P. A. Botham, 2004]. Some vital organs comprising brain, heart, lungs, liver, spleen, and kidney were harvested and weighed. Organs collected from animals were preserved in formalin solution (10%, pH7.4) for the further histopathologic examination [P. A. Botham.

### Functional Observation:

The mortality, general health status, and toxic reaction symptoms of the experimental groups were documented. Before the first day of IS 111 administration and during the experiments, a detailed clinical observation was carried out and recorded the changes in skin, eyes and mucous membrane, respiratory system, nervous system, activity, and behaviors of the rats. The body weight was measured and recorded before the first day of administration.

### Statistical Analysis:

For the acute toxicity study, the body weight data measured on Days 1, 7 and 14 were subjected to statistical assessment using one-way analysis of variance was performed. All parameters measured were analyzed statistically, apart from the general condition of the mice and findings of the macroscopic and histopathological examinations. The results were expressed as mean±SEM of the groups and significance difference between groups was evaluated by using ANOVA. If ANOVA shows significant differences, post hoc analysis was performed with Dunnett's test. The differences were considered statistically significant when p<0.01 and denoted *** as data were analyzed using Graph Pad Prism 8.2/9.0 software.

### Molecular Docking Studies of Peptide IS 111 Against Five Targets Involved in Inflammatory Process:

The molecular docking studies were performed in Maestro 9.8, Schrödinger, New York, U.S., all the calculations and chosen parameters were used the workflow implemented in the Maestro package. The peptide IS 111 in the prepared dataset was docked into the binding site of target protein using the Glide SP (standard precision) docking. Post-docking minimization was then implemented to optimize the ligand geometries.

### Protein Preparation and Active Site Analysis:

The target crystal structures were downloaded from PDB and preprocessed with the protein preparation workflow in the Maestro v9.6 (Schrodinger LLC, 2015). All hydrogens were added which were subsequently minimized with optimized potentials for liquid simulations (OPLS) 2005 force field and the impact molecular mechanics engine. Minimization was performed restraining the heavy atoms with the hydrogen torsion parameters turned off, to allow free rotation of the hydrogen by setting the root mean square deviation (RMSD) of 0.3 Å. Active site residues were obtained from the co-crystal ligand interactions and a 20 Å grid was generated around it.

### Ligand Preparation:

The given Peptide IS 111 was preprocessed by adding explicit hydrogen atoms, and ionizable compounds that were converted to their most probable charged forms at pH 7.0±2.0 using the LigPrep module of Schrödinger software (LigPrep, Schrödinger, LLC, New York, NY, 2017).

Targets that have co-crystal ligand were processed based on its interactions and the grid was generated around it and docked the given Peptide IS 111.

### Confirmatory In Vivo Animal Studies

### How Animals are Currently Used in Sepsis Research—Translational and Ethical Issues:

The central goal of animal models is to create reproducible systems of clinically relevant sepsis pathogenesis that mimic human disease and used for preliminary testing of potential therapeutic agents [Safiah Mai et al., 2012] Animal models are used for two main categories in the in the context of sepsis research:
1) To identify involvement and changes in physiological, molecular and/or cellular pathways, and to understand the mechanism of sepsis syndrome progression.
2) To study the efficacy/safety/pharmacokinetics of therapeutic targets for proof of concept or regulatory studies.

### Requirements for Animal Models of Sepsis to Mimic Human Sepsis:

An ideal animal model of sepsis would need to mimic the pace and severity of human sepsis (a severe and chronic syndrome), and its treatment in the ICU. Such success requires replication of the pathophysiology of sepsis, with particular emphasis on key patterns of inflammation and cardiovascular parameters: hemodynamic (warm shock followed by cold shock) and immunologic (proinflammatory stimulation, anti-inflammatory counter regulation, i.e., immune depression) stages; mimic histology findings in key organs (lung, liver, spleen, kidney, etc.) that are frequently modest; and—perhaps counterintuitively for animal modelers—exhibit variability among animals. For these mechanistic models of inflammatory and immune pathways and enhancing the translational gap are in need. Furthermore, an ideal sepsis model would be low-cost and would not cause any distress to the animals involved [Marcin F. Osuchowski et al, 2018]. The perfect model of sepsis does not exist based on these characteristics. Thus, to mimic human sepsis for the purpose of therapeutic intervention, considering the points mentioned above, the screening has to be done in two or more distinct animal models [S. Manoj Kumar et al., 2016; Wendy E. Walker, 2021 & Charles T Esmon et al., 2004], this helps in clinical trial prediction to be success.

A full harm: benefit impact assessment is important while designing the animal model. Harm relates to welfare experience of animal whilst benefit relates to value of scientific data, considering (3Rs) principles [Sudhir Verma, 2016; Manasi Nandi, 2020; Jean-Marc Cavaillon et al., 2020].

To date, most used models based on initiating agent, include administration of an inflammatory trigger (e.g., endotoxin-LPS), amicrobial trigger (e.g., infusion or instillation of exogenous bacteria or peritonitis) or co-morbidity models (e.g., surgical ‘immune barrier disruption models’: trauma plus infection). Considering regulatory guidelines, the present work involves the use of two Standard animal models of sepsis. The Comparison of two rodent sepsis models is summarized in Table 13.

### Justification and Significance of BALB/c Mice in Sepsis Studies:

The animal models of sepsis should be immunological and metabolic while designing the animal study, moreover in the in vivo environment, local humoral and cellular factors probably alter the way that cells respond to pro-inflammatory stimuli. The key information gathered from the animal studies are efficacy and safety of the lead molecules (as determined by standard regulatory tests). As a part of preclinical research for sepsis, mice are most popularly used.

At present, the most used inbred mouse strains in the laboratory are BALB/c, as they can improve the reproducibility and reliability of study results and majorly used in immunology and antitumor activity studies involves inflammation and macrophage function. BALB/c mice tend to produce a stronger humoral response than C57BL/6 mice; it is easier to induce Th2 immune response in BALB/c mice, which is very common in infectious diseases and allergic reactions [Hyun Keun Song et al., 2017].

In the present study, the two gold standard models which mimic human sepsis are bacterial (E. coli) infection induced peritonitis model and Cecal ligation and puncture (CLP) models are used in BALB/c mice of aged 8/10-12 weeks.

TABLE 13
The comparison of two in vivo animal sepsis models
used in the present study [Sudhir Verma 2016]

| Sepsis model | Type | Variability factors | Advantages |
|---|---|---|---|
| Bacterial Infection | Injectable | Bacterial load | Presence of bacterium |
| Model | | Route of administration | allows insights into |
| | | Time of infusion | mechanisms of host |
| | | Antibiotic/fluid | response to pathogens |
| | | resuscitation | |
| Cecal Ligation and | Surgical | Needle size and number | Mimics human sepsis |
| Puncture (CLP) | | of punctures. | most |
| | | Amount of cecum ligated/ | Simple procedure |
| | | necrosis induced. | Polymicrobial |
| | | Antibiotic/fluid | Prolonged and lower |
| | | resuscitation | cytokine elevation as in |
| | | Sex, age and strain | humans |

FIG. 4 shows a schematic representation of two most used sepsis models. (A) The injection of live bacteria E. coli (8739™) (Intraperitoneally). (B) The ceacal ligation and puncture (CLP) model by puncturing and ligating the cecum, faeces can reach the peritoneal cavity was established. Hypothermia is one of the illness signs in both models, and it can be evaluated by measuring body temperature. The figure was adopted from BioRender.com and modified.

### Bacterial Infection Model:

E. coli Induced peritonitis sepsis model. Because the rate of positive blood cultures is associated with increasing sepsis severity, (sepsis [17%], severe sepsis [25%], septic shock [69%]), it has been suggested that bacteremia plays an important role in the outcome of sepsis. Different aerobic bacterial species have been investigated to induce sepsis and septic shock. Escherichia coli is the most common one. Bacterial infection model involves the injection of live bacteria inside the body against which immune response is elicited. Animal inoculation with pure or mixed bacterial flora has long been used to explore septic processes [Wendy E. Walker et al., 2021]. There is a wide variability in the dose of and duration of infusion, as seen with endotoxicosis models. Low doses of E. coli administered over several hours in small animals have been associated with minimal early physiological changes, whereas higher doses have frequently resulted in a biphasic response, with an early rise and late fall in cardiovascular collapse and subsequent organ dysfunction and death.

### Cecal Ligation and Puncture (CLP) Model: Polymicrobial Sepsis:

The CLP model is one of the most stringent clinically applicable models of sepsis, involve a localized infection, such as surgically induced polymicrobial sepsis, that gradually propagates a systemic immune response, compared to other models, CLP provides a better representation of the complexity of human sepsis and is the crucial pre-clinical test for any new treatment to human sepsis. CLP involves a combination of three insults: tissue trauma from laparotomy, necrosis from cecal ligation, and endogenous infection from microbial leaking. In this model, feces are leaking into the abdominal cavity, introducing peritonitis (FIG. 1B) and further followed by translocation of bacteria into bloodstream which activates inflammatory response [Wichterman, Baue, and Chaudry, 1980].

The advantage of CLP is that the pathogens are endogenous, simulating severe damage that leads to peritonitis in humans. Furthermore, the course of sepsis is strikingly comparable to that of human sepsis, with both hyper- and hypo-inflammatory responses [Rittirsch D, 2009; Wendy E. Walker, 2021].

Being one of the best representatives of human sepsis, it has been recognized as the gold standard for sepsis research [Dejager L, 2011].

### Cecal Ligation and Puncture (CLP) Model: Induction of Poly Microbial Sepsis:

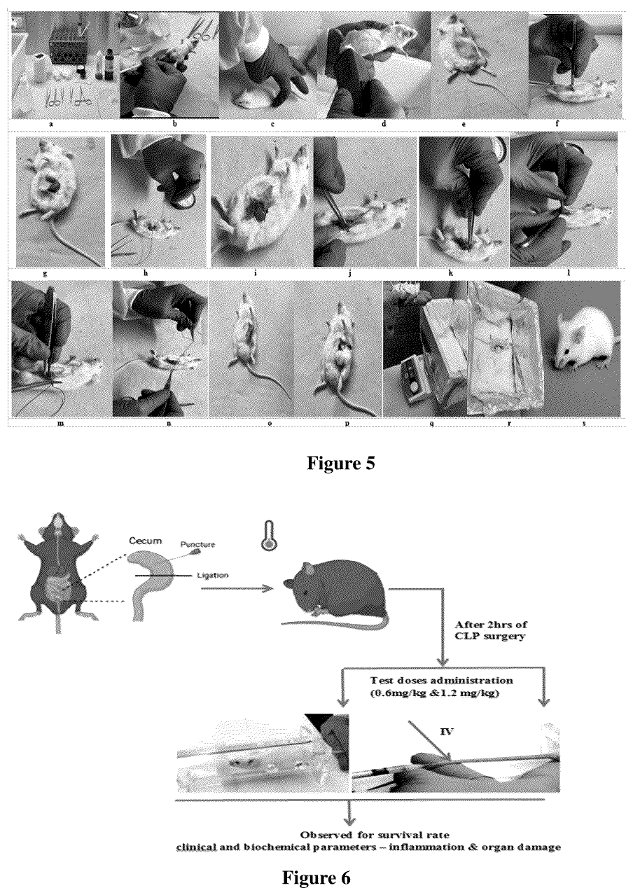

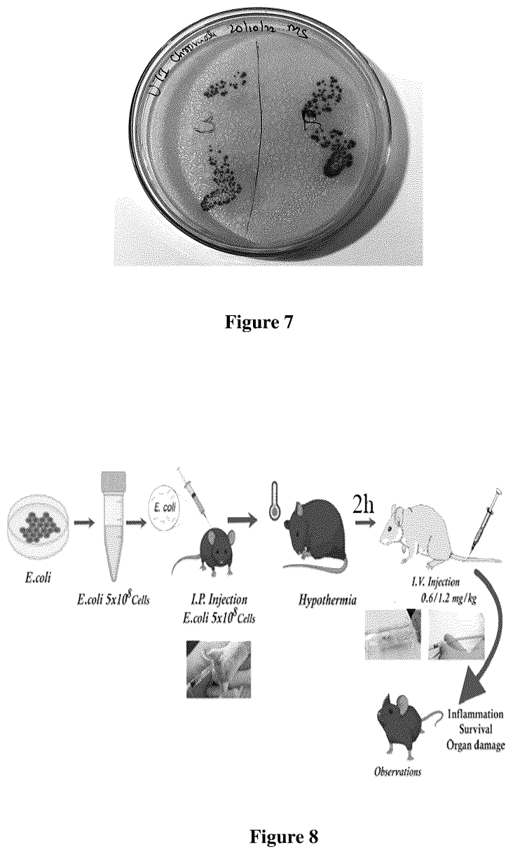

The mice were subjected to the CLP in the sequence shown in FIG. 5. The step wise procedure of the Cecum Ligation and Puncture (CLP) model involves: (a) Setup of the surgery table. (b) IP injection of anesthesia. (c) Confirmation of anesthesia by touch. (d) Shaving the surgery part of the mice. (e) Placing the mice on the surgery table and disinfecting the surgical part of the mice. (f) Skin incision. (g) Locating and exposing the cecum. (h&i) Ligated the cecum. (j) Puncturing the cecum with a 20-gauge needle. (k) Extruding the cecal matter/suspension. (l) Replacing the cecum back into peritoneal sac of the mice. (m&n) Skin closing suturing steps. (o) After the suturing. (p) Applying the betadine to the surgical part of the mice. (q & r) Placing the mice on the heating pad for the maintained of the body temperature and recovery of the anesthesia. (s) Post operated surgical mice after recovery from anesthesia.

Polymicrobial sepsis was induced using the CLP method described by [Wendy E. Walker, 2021; Benjamim et al. 2000], with minor modifications and the anesthetic used. Initially, the Mice were derived of food for 12 h before the CLP procedure and then anesthetized by intraperitoneal injection of ketamine (80-100 mg/kg BW) and xylazine in a dose (5-10 mg/kg) (for 20 g mice ketamine of 0.2 ml and xylazine of 0.1 ml). according to [Machado et al. 2012]. under sterile conditions a midline abdominal incision was made and then, a laparotomy of 10 mm/1 cm was performed, where the abdomen of mice was routinely disinfected and cut open in the middle to expose the abdominal cavity, and the cecum was mobilized, was then ligated below the cecal valve at the distal end of the cecum, ¾ was ligated with a sterile No. 4 thread, and punctured (CLP) with a 20-gauge needle in 2 places at the center of the distal end of the occlusal cecum and squeezed gently to extrude a small amount of intestinal contents through the perforation site to induce sepsis. The cecum was located but not ligated or punctured in the control animals. The cecum was placed back into the peritoneal cavity, and the abdominal incision was then closed in two layers with absorbable ethilon suture 5.0, and the animals were resuscitated with 0.1-0.2 mL of normal saline by subcutaneous injection. The sham group of mice had the identical operation as the treated and disease control groups, which included opening the peritoneum and exposing the bowel but without ligation or needle perforation of the cecum. Betadine/tramadol was used to relieve the pain of mice after operation. During the surgery, the atropine solution was applied to the eyes to prevent dryness of the eyes. All the animals were returned to their cages, where they had free access to food and water. After 2 h of CLP, the treatment groups were administered with test peptides (0.6 & 1.2 mg/kg) and the standard drug through the subcutaneous & Intravenous route. To reduce variability between studies, the CLP method was always carried out by the same investigators. [Zingarelli B et al., Libert C et al., Hellman J et al., 2019].

### Experimental Design and Treatment Groups

Mice were subjected to CLP surgery and were discarded if they died during the procedure. In all the animal experiments, the test peptide IS 111 was administered by injections respectively; According to the clinical dosage regimen, IS 111 was repeatedly administered at 2 h after CLP surgery and recovery of animals from anesthesia. The mice were divided into the following treatment groups at random: BALB/c mice are used for Intravenous (IV) route of administration. In each group the half of the animals were killed after 4 and 18 hours, [Hubbard, W. J 2005; Rittirsch D 2009]. In another half of the animals, IS 111 at two doses 0.6 & 1.2 mg/kg were injected IV daily for 5 days following CLP. 0.9% Normal saline was administered in the sham group. The survival of mice was monitored for up to 18 h and the other half of the animals are continued for the survival monitored up to 7 days.

TABLE 14
Allocating animals. dosing paradigms per group and
the study parameters of the study -CLP model.

| | | | No. of | |
|---|---|---|---|---|
| S. No | Group | Dose | animals# | Study Parameters |
| 1 | Normal control | NA | 06 | Survival Rate |
| 2 | Sham control | 0.9% NS | 12 | Physical activity: By |
| 3 | Disease control | NA | 12 | scoring system |
| | (CLP) | | | Estimation of Cytokine |
| 4 | CLP + IS 111 | 0.6 mg/Kg | 12 | Levels (Pro |
| 5 | CLP + IS 111 | 1.2 mg/Kg | 12 | inflammatory and Anti- |
| | | | | inflammatory): TNF-α, |
| | | | | IL-6, IL-1β & IL-10 in |
| | | | | serum and Peritoneal |
| | | | | Lavage Fluid. |
| | | | | Histopathology |

#The no. of animals were divided into two time points: 1) at 18 h and 2) 7 days for survial rate, except the control group. The control group animals are considered the same for all two time points.

FIG. 6 shows the study design and treatment regimen in CLP model.

### Post-Surgical Procedures:

To prevent post-surgical infection, the mice were placed in the cage supine position until the anesthetic effect wore off. To prevent hypothermia after surgery, the cage was warmed with a heater.

### Experiment 1 Survival Study:

In the survival study, survival rates were determined by setting the day 0 from 0 to 18 h after surgery and continuing observation until day 7. In one set of animals/group, survival was monitored every 4 h up to 18 h and in another half subset of animals was followed for 5 days for survival assessment every 12 h after sepsis induction to verify mortality rates. The time of death was recorded as accurately as possible.

Bacterial Infection Model: E. coli-Induced Septic Peritonitis Model

The bacterial infection model used in this study was E. coli-induced septic peritonitis, which is Murine lethal infection by intraperitoneal injection of live bacteria E. coli. [Asma Ahmed et al., 2018]

### Preparation of Bacterial Suspension:

Bacterial strain of E. coli ATCC 8739™ were maintained in our laboratory and used for the mouse sepsis model. The strain was previously stored at −80° C. in a Luria Bertani (LB) broth with 15-20% of glycerol. Single colonies from viable, growing Luria-Bertani (LB) agar plates were transferred to sterile liquid LB medium (containing 10 g tryptone, 10 g NaCl, and 5 g yeast extract per liter) and cultivated aerobically in 50-ml volumes at 37° C. in an orbital shaker for 24 h at 37° C. These cultures were transferred to 500 ml of fresh LB medium for another 12 h. The optical density (OD) of the suspension was adjusted to 0.3 (exponential phase) using a spectrophotometer. Such OD corresponds to*10⁸ colony-forming units (CFU)/mL. When bacteria were in the log phase of growth, the suspension was harvested by centrifugation at 1500×g for 5 min at 4° C.; the supernatant was discarded and washed and resuspended three times in phosphate-buffered saline (PBS) at pH 7.4 and mixed by vortexing to achieve a concentration of approximately 1×10⁸ colony-formation units (CFU) per milliliter. Finally, the bacterial suspensions were incubated in a water bath at 100° C. for 30 min to inactivate the cells. One hundred microliters of the suspension were serially diluted with sterile saline solution, plated on sheep blood agar plates, and incubated for 18 hours for determination of the bacterial concentration. The rest of the culture suspension was stored at 4° C. until use. Just before the intraperitoneal injection, the bacterial suspension was adjusted to a concentration of 1×10⁸ colony forming units (CFU) per 0.1 mL with normal saline solution/PBS. Confirmation of E. coli strain used in the study on UTI chrome plate is shown in Figure-7.

### Infection of Peritonitis in Animals:

This study was carried out within an animal containment BSL2 facility. The animals were infected with suspensions containing 5×10⁸ CFU/mL E. coli 8739™ (200 μL) inoculated intraperitoneally. The day of challenge was designated as day 1 of the experiment and considers it as 0 hr. [Wendy E. Walker, 2021; Komal Dolasia et al., 2018]

E. coli-Induced Septic Peritonitis Model:

Mice received an intraperitoneal injection of E. coli ATCC 8739, 5.0×10⁸ CFU per mouse in 200 μL to induce septic peritonitis as mentioned above. After 2 h of the E. coli infection, mice (n=6) were treated intravenously with two test doses of IS 111 (0.6 mg/kg & 1.2 mg/kg) in treatment groups. Control animals received 200 μL of normal saline. FIG. 8 shows the study design and treatment regimen in E. coli-induced septic peritonitis model. The figure was adopted and modified according to the present study. [Henrique G et al., 2020].

### Experimental Design:

In all the animal experiments, IS 111 were administered by intravenous injections respectively; According to the clinical dosage regimen, IS 111 was repeatedly administered at 2 h after the intraperitoneal injection of heat-killed E. coli. The survival of mice was monitored for up to 5 days. In each group the half of the animals were killed after 18 hours. And in other subset of the animals, IS 111 at two doses 0.6 & 1.2 mg/kg were injected IV daily for 3 days following infection. The survival of mice was monitored for up to 18 h and the other half of the animals are continued for the survival monitored up to 5 days.

TABLE 15
Allocation of animals per group & dosing
paradigms and the study parameters of the study -
study -E. coli induced peritonitis model

| S. | | | No. of | |
|---|---|---|---|---|
| No | Group | Dose | animals | Study Parameters |
| 1 | Normal control | NA | 06 | Survival Rate |
| 2 | Disease control | 0.9% NS | 12 | Physical activity: By |
| | Injection of heat- | | | scoring system |
| | killed E. coli | | | Estimation of Cytokine |
| | (5 × 10⁴ CFU per | | | Levels (Pro inflammatory |
| | mice) | | | and Anti-inflammatory): |
| 3 | Disease | 0.6 mg/Kg | 12 | TNF-α, IL-6, IL-1β & IL- |
| | Induction + IS | | | 10 in serum and Peritoneal |
| | 111 -0.6 mg/kg | | | Lavage Fluid. |
| 4 | Disease | 1.2 mg/Kg | 12 | Histopathology |
| | Induction + IS | | | |
| | 111-1.2 smg/kg | | | |

#The no. of animals were divided into two time points: 1) at 18 h and 2) 5 days for survival rate, except the control group. The control group animals are considered the same for all two time points.
The dose calculations are mentioned later in the specification.

### Experiment 1: Survival Study—

In the survival study, survival rates were determined over a period of 5 d with assessment every 12 h, by setting the day 0 from 0 to 18 h after surgery and continuing observation until day 7. In each group the half of the animals were killed after 18 hours, in another half subset of animals was followed for 5 days for survival assessment after sepsis induction to verify mortality rates. IS 111 at two doses 0.6 & 1.2 mg/kg were injected IV daily 24 h for 3 days following the E. coli Infection.

The animals were observed for 18 h and 5 days after the E. coli infection and the remaining animals are euthanized under humane conditions, the mice were anesthetized (ketamine (80-100 mg/kg BW) and xylazine in a dose (5-10 mg/kg) intraperitoneally for blood and peritoneal lavage fluid collection and euthanized with an overdose of (150 mg/kg ketamine hydrochloride and 120 mg/kg xylazine hydrochloride) for organ collection.

### Disease Parameters:

The disease parameters are observed, and procedures followed same in both the studies CLP and E. coli animal models as mentioned below [Shrum B, 2014].

### Vital Clinical Symptoms Evaluation:

The physical activity of the mice was recorded independently by two independent observers who were blinded to treatment before sampling as previously described. In this study predetermined grading system was used with a scale of 1 (healthy) to 5 (agony). This scoring system is based on grading physical activity and food intake (table 16) using spontaneous activity of mice, reactivity to external stimuli, and spontaneous food intake to differentiate between grades 1 and 5. Animals were closely observed for first 4-18 h for the development of symptoms, and where appropriate, time to death was recorded. Mice were observed at least every 6 h and for next over a 5-day period for the development of symptoms, and where appropriate, time to death was recorded. The mice that were moribund and those with a body temperature less than 27° C. were euthanized and counted as dead at each time point indicated. Malaise, immobility, and ruffled coat were noted in some animals. Humane endpoints were strictly observed (immobility, dyspnea, paralysis) so that no animal became distressed. The clinical symptoms, such as conjunctivitis, ruffling of fur coats, and activity on stimulation, were observed in all groups and graded as described earlier. A collective grade was assigned to each group depending on the severity of the symptom observed in most of the animals.

The clinical score was calculated using a score system of 0-4 grade scored as follows: grade 0, normal; grade 1, slightly lethargic; grade 2, lethargic and hunched; grade 3, very lethargic, hunched, and shaky; and grade 4, dead. Body temperature was compared for physiological evaluation.

TABLE 16
Scoring system for measuring mice physical activity:

| Grade | Quality | Criteria |
|---|---|---|
| 1 | Very active | Strong, curious, quick movements, normal |
| | | food intake |
| 2 | Active | Strong, curious, occasional interruptions in |
| | | movements, normal food intake |
| 3 | Less active | Adequate response to environment, frequent |
| | | interruptions in movements, slight decreased |
| | | food intake |
| 4 | Slow | Sleepy, slow activity, severely decreased food |
| | | intake |
| 5 | Lethargic | No activity, motionless, no food intake |

### Experiment 2: Acute Hyper Inflammation Study:

In this study, pro and anti-inflammatory cytokines TNF-α, IL-6, IL-1β, IL-12 and IL-10 in the serum and peritoneal lavage was measured.

### Quantification of Cytokine Levels:

The R&D Systems (Minneapolis, MN) IL-1β, TNF-α, IL-12 p70, IL-6 and IL-10 levels ELISA kits were used for the quantitative measurement of these cytokines either in mice sera and peritoneal exudate cell (PEC) supernatants according to the manufacturer's instructions. The results are expressed as picogram per millilitre (pg/mL) of samples analyzed.

### Experiment 3: Late Immune Paralysis Studies:

In the immune paralysis study, cytokines are estimated in PLF. [Kieslichova E, et al., 2013]

### Sampling Time Points:

The animals were observed for 18 h and 5/7 days after the Induction of disease and the remaining animals are euthanized under humane conditions, the mice were anesthetized with ketamine (80-100 mg/kg BW) and xylazine in a dose (5-10 mg/kg) intraperitoneally for blood and peritoneal lavage fluid collection and euthanized with an overdose of (150 mg/kg ketamine hydrochloride and 120 mg/kg xylazine hydrochloride)/isoflurane for organ collection. For clinical observations, the time points and parameters are listed as follows (Table 17).

TABLE 17
List of the parameters screened for
biochemical and clinical analysis.

| Time point | Parameter | Technique |
|---|---|---|
| 18^{th} hour | Estimation of cytokines in blood and | ELIZA |
| | peritoneal fluid. | |
| | Histopathology | |
| Day 5/7 | Survival rate | |

### Experimental Outcomes:

### Blood Sampling—

The whole blood and tissues were collected at 18 h and on the last day of post CLP procedure in the surviving animals for hematologic and biochemical examination. The mice were anesthetized with ketamine (80-100 mg/kg BW) and xylazine in a dose (5-10 mg/kg) at a 2:1 ratio via an intraperitoneal injection). Mice were bled retro-orbitally at specific times in either 4% EDTA or 3.2% sodium citrate to collect whole blood and plasma, respectively and was left to coagulate at room temperature for ~2 h and was centrifuged at 2,000 rpm for 10 min to separate the plasma and the serum was obtained by centrifugation at 1500 rpm for 10-15 min aliquoted and stored at −20° C. until further use in ELISA.

### Peritoneal Fluid Collection:

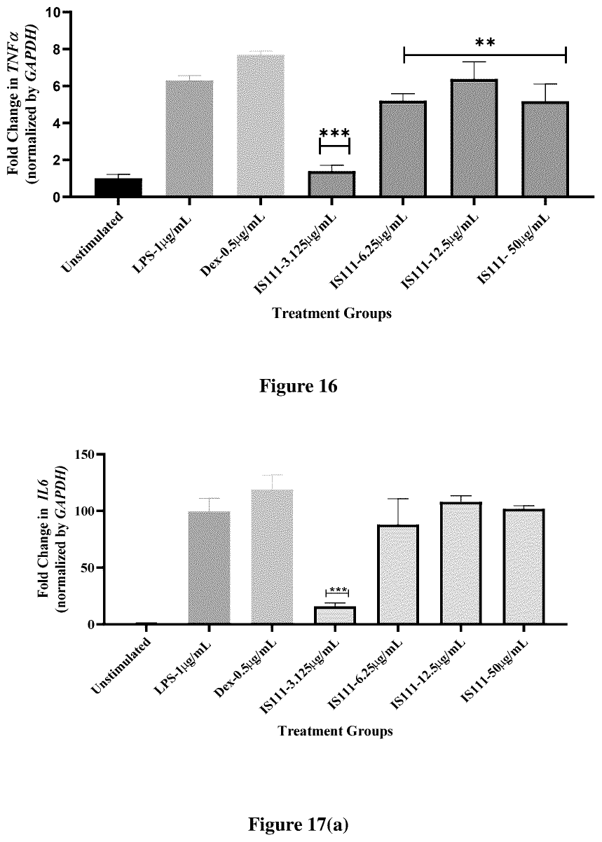

For some experiments, the peritoneal cavity of euthanized mice was washed with 5 ml sterile ice-cold PBS using an 18-gauge needle, and peritoneal lavage fluid was collected in sterile tubes and immediately placed on ice. Clear lavage was obtained by sequential centrifugation first at 2500 rpm to remove mouse peritoneal cells and 10-100 μL was used for bacterial count and next at 5000 rpm to remove E. coli bacteria. Peritoneal cell free clear supernatant was used for cell count and ELISA and cell pellet was used for Western blotting and stored at −80° C. until for further analysis (FIG. 16).

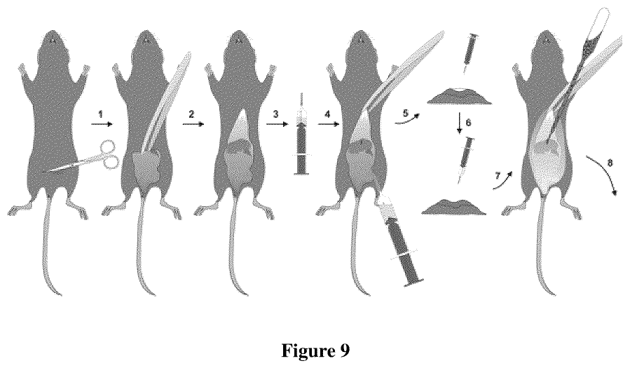

The goals of the present invention were (1) to measure bacterial growth and cell counts in peritoneal lavage fluid of anesthetized mice and (2) to investigate the direct influence of pro and anti-inflammatory cytokines (IL-1β, TNF-α, IL-12 p70, IL-6 and IL-10) in peritoneal lavage fluid [Stefan Wirtz et al., 2006]. FIG. 9 shows the collection of peritoneal lavages from the mice. [Meurer S K, 2016].

### Histopathological Evaluation: Macro- and Microscopic Assessment of the Organs:

The animals remained till the end of the study (at least n=3 for each group) and were subjected to histopathological evaluation in in vivo experiments. Some vital organs comprising Heart, Brain, Lung, Liver, kidney, and spleen were harvested and weighed, immediately after blood sample collection. The macroscopic analysis of the collected organs considered the following parameters: size, weight, integrity, and presence or not of changes visually detectable. Relative organ weight was calculated according to the following formula:

$$Relative organ weight \% = organ weight / body weight \times 100$$

For microscopic assessment, organs were fixed in formalin solution (10%, pH7.4) for the further histopathologic examination. The representative of one animal per group was presented.

### Hematoxylin-Eosin (H&E) Staining:

The organs were collected, fixed with 10% formalin, embedded in paraffin, and sectioned at a 5 μm-thickness were cut on a microtome and stained with hematoxylin and eosin. The sections were immersed in xylene I for 20 min xylene II 20 min, absolute ethanol I 5 min, absolute ethanol II 5 min, and 75% alcohol 5 min to be dewaxed and rehydrated. After rinsing with tap water, the sections were stained with hematoxylin for 3-5 min, blued, dehydrated in increasing concentrations of alcohol (85% and 95%) for 5 min, and counterstained with eosin for 5 min. Then, the sections were cleared with absolute ethanol I for five minutes, absolute ethanol II for five minutes, absolute ethanol III for five minutes, and xylene I and xylene II for five minutes each. The sections were mounted with neutral gum and graphed with 100×/200× lenses on a light microscope. At least 10 different fields were analyzed for each mouse. Pathologists who did not know about the experiment observed and scored damage to the spleen, lungs, liver, kidneys, heart, and brain.

### Histopathologic Observation:

The total surface of the slides was examined and scored by pathologist who was unaware of the groups. Briefly, the criteria for scoring lung inflammation were as follows Murakami's technique: Lung injury was assessed based on pulmonary edema as determined by alveolar wall thickening with vascular congestion and interstitial and alveolar leukocyte infiltration. Briefly, lung parenchyma was graded on a scale of 0-4 (0, absent and appears normal tissue; 1, light; 2, moderate; 3, strong; 4, intense) for congestion, edema, inflammation, and hemorrhage. A mean score for each of the parameters was then calculated. Lung injury scores were determined by assessing neutrophil infiltration, hemorrhage, necrosis, congestion, and edema as previously described. These criteria were scored as follows; 0=normal, 1≤25%, 2=25-50%, 3=50-75%, and 4≥75%.

To score liver injury, the following parameters were analyzed (16): interstitial inflammation, formation of thrombi, hepatocellular necrosis, and portal inflammation.

### Statistical Analysis:

Statistical analysis and data management were performed following recommendations on experimental design and analysis in pharmacology and all experiments were blind and based on randomly assigned groups. Data were presented as the mean±SEM of n=5 to 6 animals in each group.

The survival of the groups was estimated using a Kaplan-Meier analysis. Comparisons were performed by the log rank test. The other experiments were analyzed using one-way ANOVA for comparison within groups under univariate conditions. For multi group analysis, intergroup comparisons were performed via a Dunett's t test or Bonferroni multiple comparison test. Data were analyzed by Graph pad Prism 8.2/9.0 (Graph pad Software, La Jolla, CA, USA). P<0.001, P, 0.01 & p<0.05 was considered as statistically significant and denoted as ***, ** & * and ^{###}, ^{##} & ^{#} compared to disease control and sham control groups respectively.

The peptides IS111 of present invention can occur in form of variant thereof. The variant is a functionally active variant and may be obtained by changing sequence of IS 111 and is characterized by having a biological activity similar to that displayed by IS 111 of SEQ. ID NO. 1 from which the variant is derived. The variant includes ability of IS 111 for treatment, prevention and/or amelioration of one or more symptoms of pigmentary disorders. The functionally active variant of IS 111 protein may be obtained by sequence alterations in sequence of IS 111, wherein the peptide with the sequence alterations retains function of the unaltered peptide. Such sequence alterations can include, but are not limited to, (conservative) substitutions, deletions, mutations and insertions. The variant can comprise at least 80% of the sequence of IS 111, preferably at least 85%, still more preferably at least 90%, even more preferably at least 95% and most preferably at least 97%, 98% or 99%. The variant is derived from the IS 111 by at least one amino acid substitution and/or deletion, wherein the functionally active variant has a sequence identity to IS 111 of at least 80%, more preferably at least 85%, still more preferably at least 90%, even more preferably at least 95% and most preferably at least 97%, 98% or 99%. The variant of IS 111 is functionally active in the context of the present invention, if the activity of the variant amounts to at least 10%, preferably at least 25%, more preferably at least 50%, even more preferably at least 70%, still more preferably at least 80%, especially at least 90%, particularly at least 95%, most preferably at least 99% of the activity of IS 111 without sequence alteration. The activity of the variant may be determined or measured as described in the examples and then compared to that obtained for IS 111 of the present invention.

### EXAMPLES

Some specific examples employing peptides or variant thereof of the present invention as applicable to the above embodiments will be further described below with reference to the accompanying drawings.

### Example 1—Confirmation of Mass and Purity of Synthetic Test Peptide IS 111

The peptide has the chemical structure as provided below and details of the peptide are provided herein:

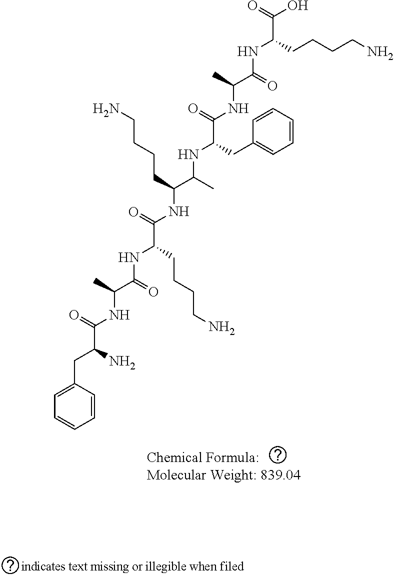 [SMILES: CC(N[C@@H](Cc1ccccc1)C(=O)N[C@@H](C)C(=O)N[C@@H](CCCCN)C(=O)O)[C@H](CCCCN)NC(=O)[C@H](CCCCN)NC(=O)[C@H](C)NC(=O)[C@@H](N)Cc1ccccc1]

### The Physical and Chemical Properties of Synthetic Peptide IS 111 (SEQ ID NO 1);

Chemical Composition: 7 amino acids
Chemical Formula: C42H66N10O8
Molecular Weight: 839.04 g/mol
Sequence of the short synthetic peptide
Molecular three letter code: PHPP-Phe-ala-lys-lys-phe-ala-lys-NH2 (SEQ ID NO 1)
Molecular single letter code: FAKKFAK (SEQ ID No 1)
Molecular primary structure: linear peptide
Molecular secondary structure: β-sheet
Solubility: soluble in water

The synthetic peptides which are synthesized, and their mass and purity were confirmed by HRMS and HPLC details are as follows: The purity and the mass of the peptides confirmed at In-house and the details are mentioned in Table 18.

TABLE 18
The details of the peptides used in the present study

| | | | Molecular weight | Purity |
|---|---|---|---|---|
| S. no | Peptide code | No. of residues | (m/z) | (%) |
| 3 | IS 111 | 07 | 839.04 | 99.2 |

The certificate of analysis of all three-test peptide generated by In-house.

### HRMS Results:

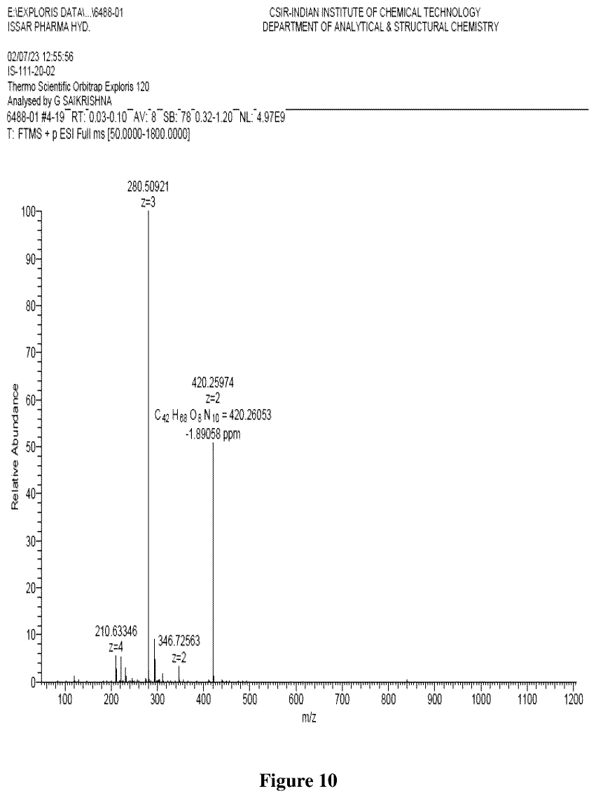

The goal of the analysis was to confirm the peptide molecular weight. However, MS confirms that only m/z=2 belong to the peptides. The details of the instrument and mobile phase used are as mentioned in Table 19 and the obtained chromatograms of the peptides are shown in FIG. 10.

TABLE 19
Conditions used for HRMS analysis

| | |
|---|---|
| Instrument name | Thermo scientific ™ Q orbitrap exploris ™ 120 mass |
| | spectrometer and couple vanquish ™ 3000 UHPLC |
| Source | Electro Spray Ionization ESI with full scan |
| Mobile phase | Water + Methanol (30&:70%) |
| Flow rate | 0.6 flowrate 0.6 ml/min |

### HPLC Analysis Results:

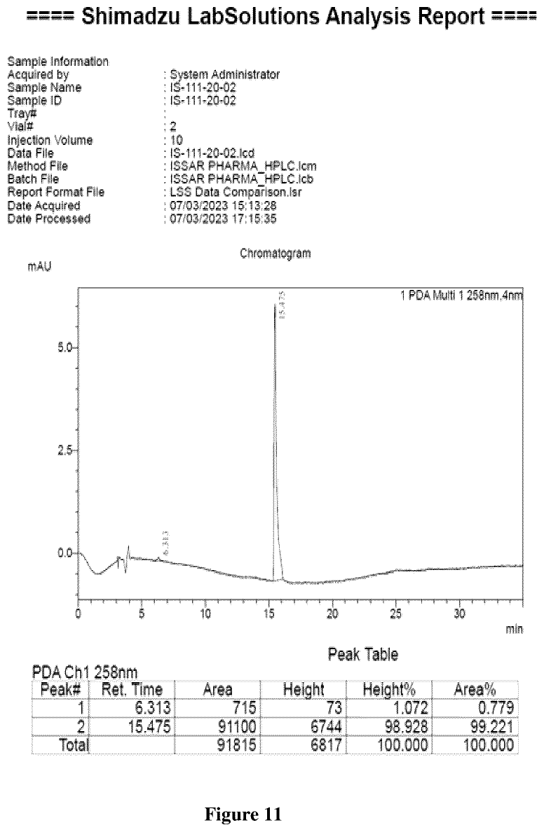

The goal of the analysis was to confirm the purity of the peptides used in the present study. The details of the instrument and mobile phase used are as mentioned in Table 20 and the obtained chromatograms of the peptides are shown in FIG. 11.

TABLE 20
Chromatographic conditions used for the peptides IS 111

| Instrument Name | HPLC Shimadzu with Lab solutions software | | |
|---|---|---|---|
| | (SPD-M20 Detector) | | |
| Column | Purosphere Star RP-18 End capped (250 × 4.6 | | |
| | mm, 5 μm) | | |
| Column Oven | ambient | | |
| Temperature | | | |
| Flow rate | 1 mL/min | | |
| Wavelength/Detector | 276 nm/PDA; 215 nm -IS 111 | | |
| Injection Volume | 10 μL | | |
| Mobile phase | MP-A: −0.1% TFA in Milli-Q water or | | |
| | equivalent grade water | | |
| | MP: -B: −0.1% TFA in ACN | | |
| | Gradient programme | | |
| | Time | MP-A (%) | MP-B (%) |
| | 0.01 | 90 | 10 |
| | 17.00 | 72 | 28 |
| | 30.00 | 72 | 28 |
| | 32.00 | 90 | 10 |
| | 35.00 | 90 | 10 |

| | |
|---|---|
| Diluent | Acetonitrile + water (50:50) |
| Run Time | 35 minutes; 31 minutes -IS 111 |

### Example 2: In Vitro Anti-Inflammatory Data of Test Peptides Against LPS-Induced Inflammation in RAW 264.7 Cells

### Effects of Test Peptide IS 111 on the Viability of RAW 264.7 Macrophages:

The test peptide IS 111 cytotoxic effects on RAW 264.7 mouse primary cells were determined by MTT assay. MTT assays were performed in RAW 264.7 cells treated with different concentrations of test peptides (1.56, 3.12, 6.25, 12.5, 25, 50 and 100 μg/mL) for 48 h. Cell viability was measured by MTT assay based on the conversion of yellow tetrazolium salt to form a purple formazan product. The viability of the RAW 264.7 cells can be seen on table 21. The MTT assay revealed that test peptides concentrations (1.56, 3.12, 6.25, 12.5, 25, 50 and 100 μg/mL) exerted no significant cytotoxicity in the RAW264.7 macrophage cells, and safe to the cells with cell viability 80%, Hence, concentrations (3.12-100 μg/mL) to be applied for the next assays.

Cell viability results revealed that treatment with peptides did not show above 80% viability at the tested concentrations.

TABLE 21
Cell viability of the test peptide IS
111 in comparison with dexamethasone

| | % of Cell viability | | |
|---|---|---|---|
| Test peptides | of test peptides | | Dexamethasone |
| concentrations | IS111 | Dexamethasone | concentrations |
| 100 | 91.37 | 84.61 | 500 |
| 50 | 94.98 | 90.795 | 250 |
| 25 | 96.39 | 95.96 | 125 |
| 12.5 | 99.72 | 91.44 | 62.5 |
| 6.25 | 90.21 | 82.80 | 31.25 |
| 3.125 | 91.55 | 90.00 | 15.625 |
| 1.5625 | 84.54 | 102.36 | 7.8125 |

### In Vitro Anti-Inflammatory Activity of Test Peptide IS 111:

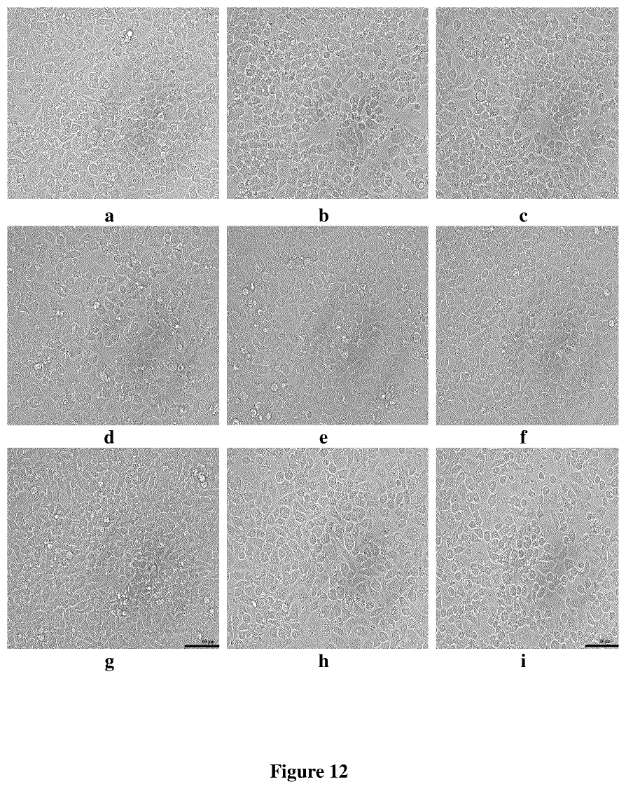

For analysis of anti-inflammatory effects, the cells were pretreated with test peptides of different concentrations (3.12, 6.25, 12.5, 25, 50 and 100 μg/mL) before incubation with LPS for 18 hours. The Morphology of macrophage RAW 264.7 cell visualized by optical microscopy at scale bar 60 μm of peptide is shown in FIG. 12.

### Effect of Early Phase Cytokines on the LPS-Stimulated RAW 264.7 Macrophages:

### The Test Peptide IS 111 Reduce the Production of Pro-Inflammatory Cytokines:

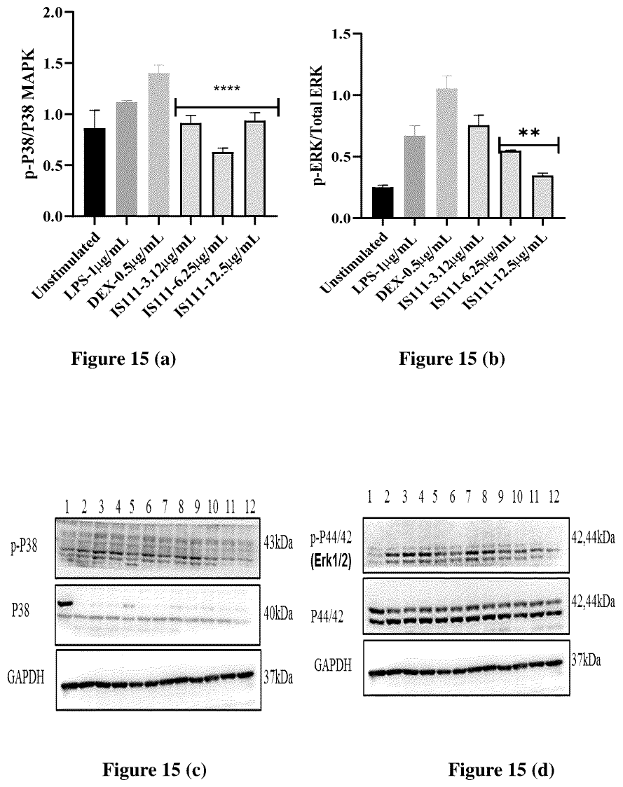

Certain HDPs can modulate the innate immune response due to their ability to stimulate the induction of chemokines while suppressing potentially harmful pro-inflammatory cytokines. In the present study, the anti-inflammatory activities of test peptide IS 111 was evaluated on murine macrophage-like RAW 264.7 cells stimulated with 10 μg/mL LPS and compared them with standard drug dexamethasone 500 μg/mL. The concentrations of TNF-α IL-6, IL-1β and IL-10 in the culture supernatants of RAW 264.7 cells were measured by an ELISA kit. Treatment of RAW 264.7 cells with LPS alone resulted in insignificant increases in cytokine production as compared to the control group. RAW 264.7 cells stimulated with LPS alone produced low levels of IL-10 while strongly inducing IL-6 and TNF-α levels. In the presence of test peptide IS 111, there was a significant increase in IL-10 production, particularly in groups treated with all test peptides compared to groups exposed to standard drug dexamethasone. All three peptides caused a significant and almost complete reduction in the release of pro-inflammatory cytokines IL-1β, IL-6 and TNF-α in LPS-stimulated cells (FIGS. 15-16, respectively). The levels of TNF-α were significantly decreased as compared to the LPS group in all test groups (**** & ***, p<0.001 &p<0.01 respectively) compared to IL-6, production TNF-α has been reduced to a greater extent. ELISA results revealed that treatment with LPS significantly increased the TNF-α, IL-1β and IL-6, whereas IL-10 levels are decreased. Treatment with IS 111 reduced the levels of IL-6 but not in a dose-dependent manner. Further, treatment with IS111 significantly increased the anti-inflammatory marker IL-10 levels. Similarly, treatment with IS111 significantly reduced the TNF-alpha levels. However, these compounds failed to show dose-dependent inhibition of IL-1β. Dexamethasone also showed significant anti-inflammatory action by inhibiting the entire marker's expression.

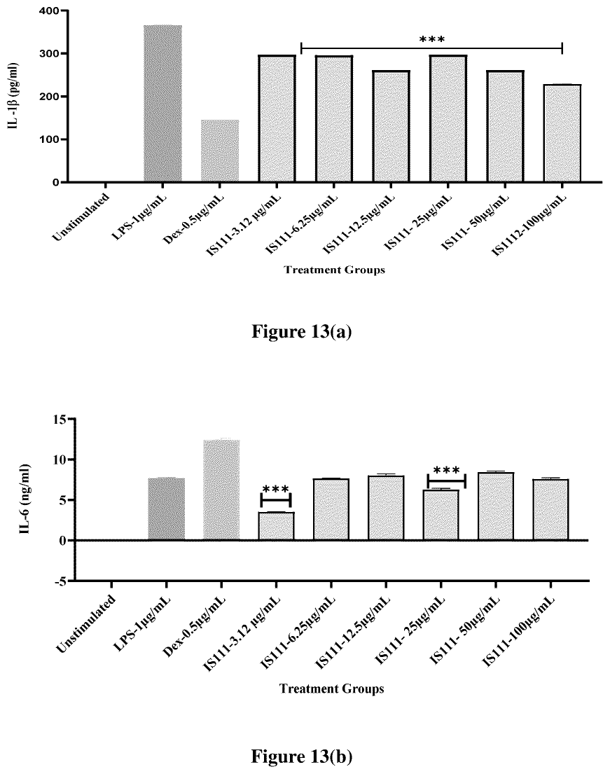

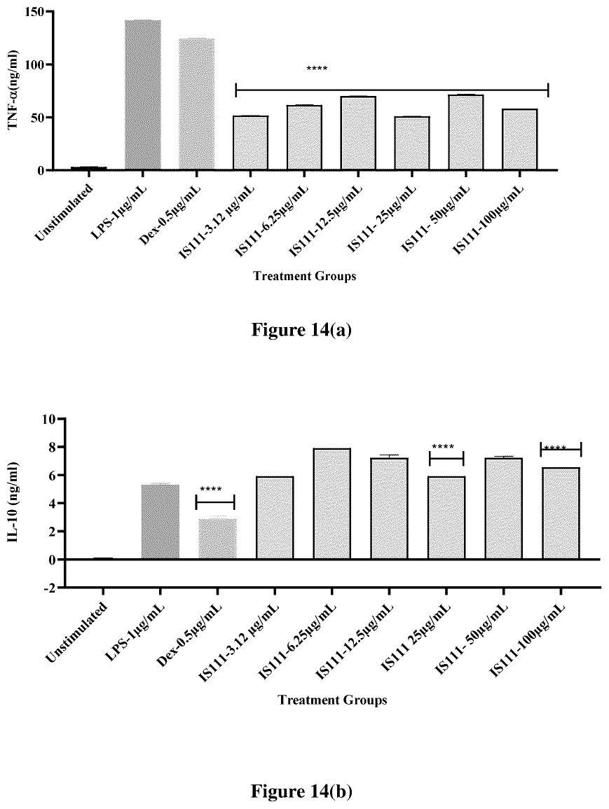

FIG. 13(a) shows IL-1β secretion measured using ELISA. FIG. 13(b) shows IL-6 secretion measured using ELISA. FIG. 14(a) shows TNF-α secretion measured using ELISA. FIG. 14(b) shows IL-10 secretion measured using ELISA.

Data is presented as fold change to LPS stimulation and unstimulated (control) cells. Data were shown as the means±SD. Each experiment was repeated in triplicate. ****p<0.001, ***p<0.01, **p<0.05, in comparison to LPS group. Bars indicate means and vertical lines standard error of mean of three independent experiments analyzed in duplicate.

### The Effects of Test Peptide IS 111 on LPS-Induced MAPK Pathways Activation:

To investigate the mechanism by which test peptide IS 111 inhibits LPS-induced production of inflammatory cytokines, western blot showed that IL-10 levels were increased in IS111 treated cells. The effect of test peptides on the LPS-induced phosphorylation of ERK1/2 and p38 MAPK in the cell lysate by Western blotting analysis using two different phospho-specific antibodies was examined. The MAPK signaling pathway is known to be important for the expression of pro-inflammatory genes in LPS-treated RAW 264.7 cells. MAPKs act as specific targets for inflammatory responses. As shown in FIG. 4, the phosphorylation level of the ERK1/2, and p38 MAPK increased dramatically after stimulation with LPS, whereas test peptides attenuated the LPS induced activation of p-ERK1/2, MAPKs in a dose dependent manner. However, the total protein expression levels of the unphosphorylated MAPKs were unaffected by LPS and test peptides treatment. The results confirmed that pretreatment with test peptides may block the LPS induced expression of pro inflammatory responses by inhibiting the MAPK signaling pathway and IS 111 significantly inhibited the phosphorylation of ERK and markedly prevented the LPS-induced increasing of p38 phosphorylation in a concentration-dependent manner [FIGS. 15(a) to (d)]. FIGS. 15(a) to (d) show the protein levels of P38 & ERK1/2 evaluated by western blot. Data is presented as fold change to LPS stimulation and unstimulated (control) cells.

Data were shown as the means±SD. Each experiment was repeated in triplicate. ****p<0.001, ***p<0.01, **p<0.05, in comparison to LPS group. Bars indicate means and vertical lines standard error of mean of three independent experiments analyzed in duplicate.

Effect of Test Peptides IS111 on mRNA Expressions of Cytokines:

Real-time PCR analyses of the mRNA levels of IL-β, IL-6, CCL2 and TNF-α were performed to provide an estimate of the relative levels of expressions of these genes. In the present study, the mRNA expression levels of IL-β, IL-6, CCL2 and TNF-α were increased in the LPS treated groups compared to the control groups and, the mRNA expression levels of IL-β, IL-6, CCL2 and TNF-α were decreased in the test peptides treated groups compared to the LPS treated groups (FIGS. 16-18). Treatment with IS111 showed reduction of CCL2 and IL-1β with dose dependent manner. Treatment with IS111 showed significant reduction of TNF-α and IL-6 compared to LPS control. IS111 dose dependently reduced the expression of IL-1β compared to LPS control.

FIG. 16 shows effect of test peptide IS 111 on TNF-α mRNA expression was detected by RT-qPCR. Data is presented as fold change to LPS stimulation and unstimulated (control) cells. Data were shown as the means±SD. Each experiment was repeated in triplicate. ****p<0.001, ***p<0.01, **p<0.05, in comparison to LPS group. Bars indicate means and vertical lines standard error of mean of three independent experiments analyzed in duplicate.

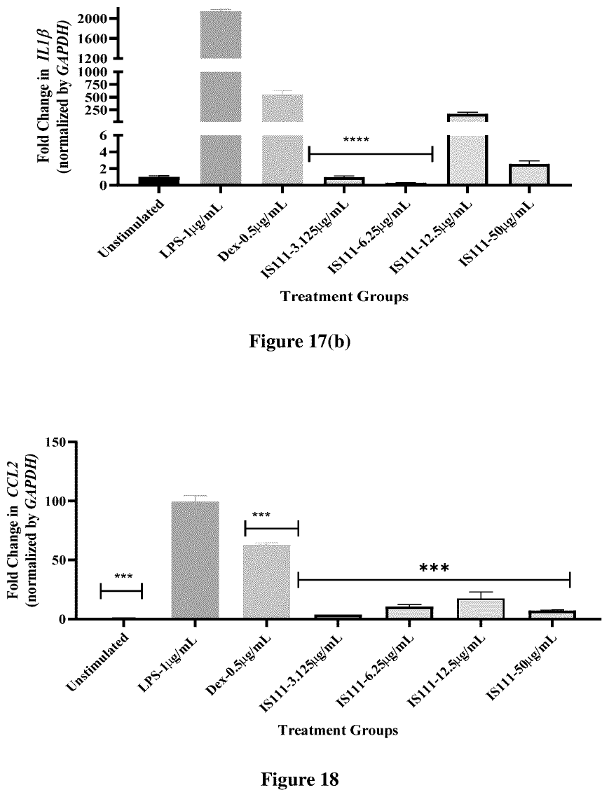

FIG. 17 (a,b) shows effect of test peptide IS 111 on IL-6 & IL-1β mRNA expression was detected by RT-qPCR. Data is presented as fold change to LPS stimulation and unstimulated (control) cells. Data were shown as the means±SD. Each experiment was repeated in triplicate.

****p<0.001, ***p<0.01, **p<0.05, in comparison to LPS group. Bars indicate means and vertical lines standard error of mean of three independent experiments analyzed in duplicate.

FIG. 18 shows effect of test peptide IS 111 on CCL2 mRNA expression was detected by RT-qPCR. The test peptides inhibit LPS-induced pro-inflammatory cytokine expression at the mRNA level. Data is presented as fold change to LPS stimulation and unstimulated (control) cells. Data were shown as the means±SD. Each experiment was repeated in triplicate. ****p<0.001, ***p<0.01, **p<0.05, in comparison to LPS group. Bars indicate means and vertical lines standard error of mean of three independent experiments analyzed in duplicate.

In vitro cell-based assays often play an important part in the preclinical screening of new lead molecules, and it is important to pay due care and attention to certain aspects of the design of cell-based assays. For example, the use of primary cell cultures might be desirable, rather than cultures of immortalized cell lines. LPS, a constituent of the outer membrane of gram-negative bacteria, apparently is one of the major toxins responsible for initiating this pathophysiological cascade to release numerous proinflammatory mediators such as TNF-α, IL-6, IL-1 and IL-12 [Jiang Z et al., 2004]. In addition to cases caused directly by gram-negative microorganisms (accounting for 30-40%) in bacteremia involving gram-positive bacteria or in systemic inflammatory response syndrome, LPS may also play a pathogenic role due to bacterial translocation from the gut.

Certain HDPs can modulate the innate immune response due to their ability to stimulate the induction of chemokines while suppressing potentially harmful pro-inflammatory cytokines. In the present study, the anti-inflammatory activity of peptide IS 111 was evaluated on murine macrophage-like RAW 264.7 cells stimulated with 10 ng·ml-1 LPS and compared them with the standard drug dexamethasone. It should be noted that this inhibitory effect was not due to cytotoxic activity of IS 111, because the cell viability was not affected by IS 111 treatments. Cell viability results revealed that treatment with peptides did not show above 80% viability at the tested concentrations.

The results of TNF-α, IL-1β and IL-6 quantification which are shown in FIG. 28-30) reveal that the test peptides IS 111 had inhibition activity towards mentioned inflammatory mediators production in LPS induced RAW 264.7, but treatment with lower concentration which is 25 μg/ml resulted in better activity compared to dose of 100 μg/mL. TNF-α, IL-1β and IL-6 level in positive control are significantly higher compared to the level of negative control which proves that LPS can increase mentioned Inflammatory mediators production in RAW 264.7 cell. In this study, test peptide IS 111 could inhibit TNF-α, IL-1β and IL-6 production in RAW 264.7 cell lines which suggest that they have anti-inflammatory effect through down regulation of those pro-inflammatory cytokines. The data revealed that the test peptide IS 111 inhibited TNF-α, IL-6 and IL-1β, production in LPS-stimulated RAW 264.7 cells [Hong Z et al., 2004] treatment with IS 111 reduced the levels of IL-6 but not in a dose-dependent manner. Similarly, treatment with test peptide IS111 significantly reduced the TNF-alpha levels. TNF-α is also a cytokine that plays a significant role in inflammation. This cytokine is produced chiefly by activated macrophages and play role during inflammatory response activating adhesion molecule inducer and nuclear factor kappa-light-chain-enhancer of activated cells (NF-κB) [An H et al., 2002]. TNF, being an endogenous pyrogenic, is able to induce fever, apoptotic cell death, cachexia, inhibit tumor genesis and viral replication, respond to sepsis via IL-1β and IL-6 producing cells.

Deregulation of TNF production has been implicated in a variety of human diseases including Alzheimer's disease, cancer, major depression, psoriasis, and inflammatory bowel disease (IBD). With all effects caused, TNF-α inhibitory activity measurement is important in anti-inflammatory potential agent screening since this cytokine is an important mediator of inflammation. TNF-α may initiate an inflammatory cascade consisting of other inflammatory cytokines, chemokines, growth factors, endothelial adhesion factors and recruiting a variety of activated cells at the site of tissue damage. It is known that TNF-α can induce DNA damage, inhibit DNA repair, and act as a growth factor for tumor cells [McCoy S L et al., 2004].

Treatment of macrophages with LPS led to significant increase in the levels of both TNF-α and nitrites in the culture supernatants relative to control levels. IL-1β is important for the initiation and increase the inflammatory response to microbial infection during inflammation process due to its role to induces secretion of proinflammatory cytokines such as IL-6 and IL-8 [Liao J C et al., 2012]. IL-6 has a wide range effect on immune system cells resulting in the acute inflammation response. Increasing of IL-6 level was reported in rheumatoid arthritis, psoriasis, and encephalomyelitis individuals.

Therefore, inhibition of IL-1β synthesis would indirectly be useful for autoimmune disease and inflammation treatment. RAW 264.7 cells stimulated with LPS alone produced low levels of IL-10 while strongly inducing TNF-α IL-1β and I-6 levels. In the presence of peptides IS 111, there was a significant increase in IL-10 (FIG. 30), production, particularly in groups treated with IS 111 significant and almost complete reduction in the release of pro-inflammatory cytokines TNF-α IL-1β and I-6 levels n LPS-stimulated cells (FIGS. 28-30 respectively). Dexamethasone also showed significant anti-inflammatory action by inhibiting the entire marker's expression [Eicher S D et al., 2004].

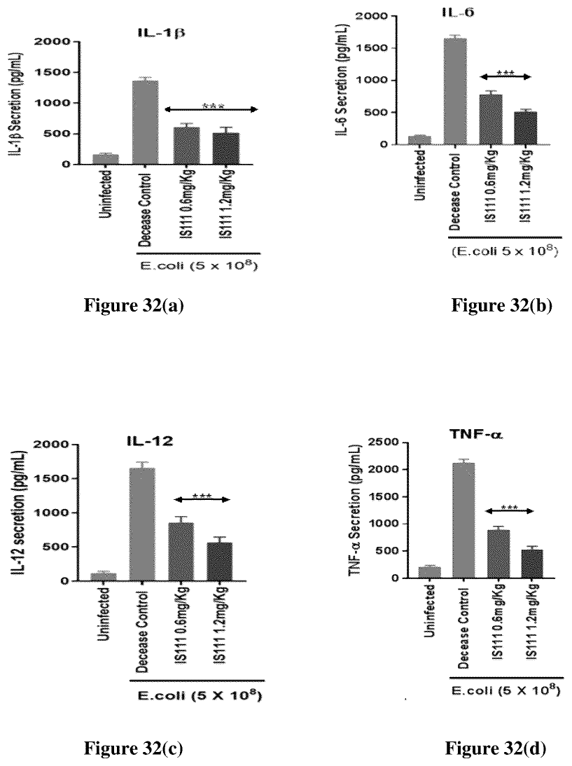

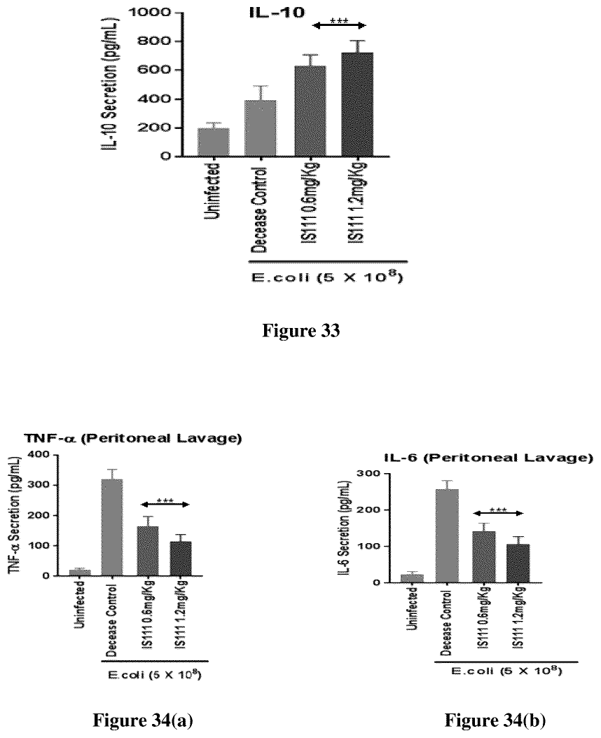

Western-blot results showed that treatment with LPS significantly increased the pERK levels, treatment with IS 111 showed significant reduction of pERK levels. The mechanisms of action underlying such a function. MAPKs signaling pathways are involved in the LPS-induced pro-inflammatory mediators and cytokines expression was also investigated, which play a critical role in the regulation of cell growth and differentiation as well as the control of cellular responses to cytokines and stresses [Chen H H et al., 2004]. Maximal MAPK expression is known to occur 20-30 min after LPS treatment in human and murine monocytes and macrophages [24]. Inhibition of any of the three MAPK pathways (JNK, p38 MAPK, and ERK) is sufficient to block induction of TNF-α by LPS. The results revealed that the test peptides (IS 111) obviously down-regulated LPS-induced phosphorylation of ERK1/2 in activated macrophage cells. This result suggests that p38 and ERK1/2 are involved in the inhibition by the test peptide IS 111 in RAW 264.7 cells (FIGS. 32-33) and participates in regulating the expression of cytokines and other mediators that are involved in the inflammatory response [Rina Barouch et al., 2001; Xia, Z et al., 1995]. Thus, inhibition of the production of these signaling pathways may explain the potent activity of the test peptide IS 111 as a suppressor of inflammatory cytokines. These results are supported by gene expression analysis. The results revealed that LPS stimulation significantly increased the expression of CCL2, TNF-α, IL-6 and IL-β compared to control. (FIG. 34-36) Treatment with IS 111 showed reductions of CCL2 and IL-1 beta with dose dependent manner. Treatment with IS111 showed significant reduction of TNF-alpha and IL-6 compared to LPS control.

The test peptide IS 111 showed significant anti-inflammatory activity against LPS induced inflammation in RAW 264.7 cells by inhibition of pro-inflammatory cytokines (CCL2, TNF-α, IL-6 and IL-β) and downregulated phosphorylation of p38 mitogen activated protein kinase (MAPK).

### Example 3: In Vitro Anti-Microbial Study

Multi drug resistance is a global health problem, probably related to millions of deaths each year [WHO, Geneva; 2014]. Nowadays, there are different “in vitro” methods to evaluate the antibacterial activity of novel test lead molecules, broth dilution method is the gold standard to determine the minimum inhibitory concentration (MIC). The anti-microbial Activity was assessed by broth microdilution and time-kill viability assays against all tested pathogenic bacteria.

The study results confirmed that the test peptide IS 111 at concentrations did not exhibit cytotoxicity at the physiologically effective antibacterial concentrations.

### Minimum Inhibitory Concentration (MIC) Assay:

In this study, MIC values were determined by the micro broth dilution method in Mueller Hinton broth procedure as described above. All MIC values calculated in the present study are listed in Table 22. IS 111 displays rapid killing activity against pathogenic bacteria. It was demonstrated that IS 111 exhibits antibacterial effects against different bacterial pathogens (MIC between 0.39 and 1.56 μg/ml).

TABLE 22
Minimum inhibitory concentration (MIC) of Peptide IS
111 and Antibiotic (Ciprofloxacin) against S. aureus,
P. aeruginosa, E. coli and K. Pneumoniae

| | | Peptide | Positive control |
|---|---|---|---|
| | | IS 111 | (Ciprofloxacin) |
| S. No | Species | (μg/mL) | (μg/mL) |
| 1 | Staphylococcus aureus | 3.13 | 1 |
| 2 | Pseudomonas aeruginosa | 3.13 | 0.5 |
| 3 | Escherichia coli | 1.56 | 0.5 |
| 4 | Klebsiella pneumoniae | 6.25 | 1 |

Results shown in the Table are Mean±SD obtained from triplicate experiments.

The anti-microbial assay results showed that the synthetic peptide IS 111 possess strong antimicrobial activity in vitro, against four pathogens and may therefore have a role as an antimicrobial agent. The time-kill studies have provided valuable information on the rate, concentration and potential action of antibacterial agents in vitro. Further time-kill test showed that this compound was strong bactericidal agents against four pathogens: Staphylococcus aureus subsp. aureus (ATCC® 6538™), Pseudomonas aeruginosa (ATCCR 9027™), Escherichia coli (ATCCR 8739™) and Klebsiella pneumoniae subsp. Pneumoniae (ATCC® 700603™).

Time-Killing Assay/Survival of Microbial Species: Time-Kill Kinetics of IS 111 Against S. aureus, P. aeruginosa, E. coli and K. pneumoniae:

The time-kill kinetics antibacterial study of the Peptide IS 111 was carried out to assess the killing rate of the compound within a given contact time. The survival of bacteria, time kill efficacy assay of IS 111 was further evaluated for activity against Escherichia coli ATCC 8739 and Staphylococcus aureus ATCC 6538, Pseudomonas aeruginosa ATCC 9027 and Klebsiella pneumoniae ATCC 700603 according to CLSI reference method, with slightly modification. MICs were determined by broth microdilution assay as described above. The test compound IS 111 was incorporated into 4.9 ml Mueller Hinton Broth (MHB) at concentrations of 0.5×MIC, 1×MIC and 5×MIC for each bacterial species, the concentration are mentioned in the Table no. 10. Test tubes of MHB without test compound Peptide IS 111 were used as vehicle controls. Overnight cultures of the bacterial strains at cell densities of approximately 1×10⁶ CFU/ml were used to inoculate both test and control tubes.

The cultures were then incubated in a shaker at 37° C. for 1, 2, 3, 4, 5, 12 and 24 h. After each interval, ten-fold dilutions were prepared with normal saline, and 100 μL incubated suspension was transferred on the agar plates and spread through the spreader. Colony counts were performed after 24 h incubation at 37° C. Plates with 10-300 colonies were used for these counts, and the kill rate was determined by plotting log 10 viable counts (CFU/ml) against time. Bactericidal activity was defined as a ≥3 log 10 decrease in CFU/ml of the initial microbial population, while bacteriostatic activity was defined as a <3 log 10 decrease in CFU/ml. The assay was performed in triplicate. The proportion of cells surviving at 60 min was calculated, as was the mean and standard deviation. Proportions of surviving cells were compared using Student's 2-tailed t-test, assuming unequal variance and differences were considered significant where p≤0.05.

TABLE 23
The MIC concentration of test peptide IS 111 and the standard
antibiotic against test species are tabulated as follows:

| | Concentration of IS 111 & Ciprofloxacin | | | | | | | |
|---|---|---|---|---|---|---|---|---|
| | MIC values used against test species | | | | | | | |
| | S. | | P. | | E. | | K. | |
| Groups | aureus | | Aeruginosa | | coli | | pneumoniae | |
| Vehicle | 0 | | 0 | | 0 | | 0 | |
| Control | | | | | | | | |
| IS 111 | 1.565 | μg/ml | 1.565 | μg/ml | 0.78 | μg/ml | 3.125 | μg/ml |
| 0.5 × MIC | | | | | | | | |
| IS 111 | 3.13 | μg/ml | 3.13 | μg/ml | 1.56 | μg/ml | 6.25 | μg/ml |
| 1 × MIC | | | | | | | | |
| IS 111 | 15.65 | μg/ml | 15.65 | μg/ml | 7.8 | μg/ml | 31.25 | μg/ml |
| 5 × MIC | | | | | | | | |
| Ciprofloxacin | 1 | μg/ml | 0.5 | μg/ml | 0.5 | μg/ml | 1 | μg/ml |
| 1 × MIC | | | | | | | | |

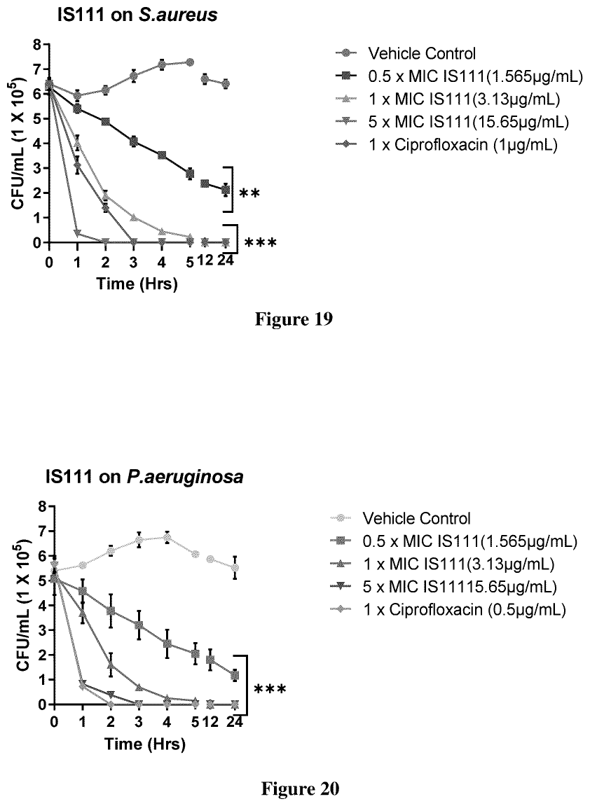

The time-kill assays have been widely used for in vitro investigations of new antimicrobial agents as these provide descriptive (qualitative) information on the pharmacodynamics of antimicrobial agents [Olajuyigbe & Afolayan, 2012] and can be classified as bacteriostatic or bactericidal, based on the characterization of the relationship between agent concentration and activity over time. IS111 displays rapid killing activity against pathogenic bacteria. In the present study, time-kill assays were performed to analyze the killing rate of IS 111 and to compare it with that of conventional antibiotic “Ciprofloxacin” which is frequently used in clinical settings. MIC values for IS 111 and selected antibiotic (Ciprofloxacin) were determined and are listed in Table 23. The increase in viable count of bacteria in the control group shows these bacteria were actively growing from 1 to 24 h. FIGS. 19-22 (shows the killing curves of IS 111 and Ciprofloxacin for S. aureus, P. aeruginosa, E. coli and K. pneumoniae [Boorn K L, et al., 2010; Pradhan, S., et al., 2020].
Time-Kill Kinetics of Peptide IS 111 Against S. aureus, P. Aeruginosa, E. Coli and K. pneumoniae:

Time-kill assays allow antibacterial agents to be classified as bacteriostatic or bactericidal, and characterization of the relationship between agent concentration and activity over time. IS111 displays rapid killing activity against pathogenic bacteria. In the present study, time-kill assays were performed to analyze the killing rate of Peptide IS 111 and to compare it with that of conventional antibiotic “Ciprofloxacin” which is frequently used in clinical settings. MIC values for IS 111 and selected antibiotic (Ciprofloxacin) were determined and are listed in Table 22. The increase in viable count of bacteria in the control group shows these bacteria were actively growing from 1 to 24 h. FIGS. 19-22 shows the killing curves of peptide IS 111 and Ciprofloxacin for S. aureus, P. aeruginosa, E. coli and K. pneumoniae.

Time-Kill Kinetics of Test Peptide IS 111 Against S. aureus:

The result obtained with the test compound IS 111 and S. aureus was shown in FIG. 19. After 24 h incubation with 0.5×MIC (1.56 μg/ml) of IS 111, a 2 log 10 CFU/ml reduction in viability of S. aureus occurred, indicating the IS 111 was bacteriostatic against this strain. At a concentration of 1×MIC (3.13 μg/ml), however, the IS 111 was bacteriostatic against S. aureus by 12 h. Also, increasing the concentration to 5×MIC (15.6 μg/ml) resulted in bacteriostatic activity against S. aureus by 2 h and the Complete Reduction of Initial inoculum of S. aureus was achieved. These results indicate that anti-bacterial activity of IS 111 was both time- and concentration-dependent. In comparison, the killing activity of Ciprofloxacin at 1×MIC was slower and Complete Reduction of Initial inoculum of S. aureus was achieved by 3 h treatment (FIG. 19 & Table 24). At 3 h, the Peptide IS 111 and ciprofloxacin display similar killing activities.

FIG. 19 shows the Time-kill kinetics of test peptide IS 111 against S. aureus. Results shown in the table are Mean±SEM obtained from triplicate experiments where P<0.005, P<0.05 and denoted as *** & ** when compared with vehicle control group respectively. Time-kill profiles for S. aureus in Trypticase soya agar during treatment with 1.565 μg/ml (0.5×MIC), 3.13 μg/ml (1×MIC) and 15.65 μg/ml (5×MIC) IS 111 and Ciprofloxacin 1 μg/ml (1×MIC) as a standard.

TABLE 24
Time Kill Kinetics of test peptide IS 111 against S. aureus:

| | Viable count at different time of exposure (1 × 10⁵ CFU/ml) | | | | | | | |
|---|---|---|---|---|---|---|---|---|
| Groups | 0 h | 1 h | 2 h | 3 h | 4 h | 5 h | 12 h | 24 h |
| Vehicle | 5.4 ± 0.35 | 5.6 ± 0.13 | 6.2 ± 0.20 | 6.7 ± 0.30 | 6.8 ± 0.23 | 6.1 ± 0.08 | 5.9 ± 0.08 | 5.5 ± 0.45 |
| Control | | | | | | | | |
| IS111 | 5.1 ± 0.65 | 4.6 ± 0.48 | 3.8 ± 0.68 | 3.2 ± 0.58 | 2.5 ± 0.58 | 2.1 ± 0.43 | 1.8 ± 0.43 | 1.2 ± 0.23 |
| 0.5 × MIC | | | | | | | | |
| IS111 | 5.3 ± 0.10 | 3.7 ± 0.43 | 1.6 ± 0.48 | 0.7 ± 0.15 | 0.3 ± 0.15 | 0.2 ± 0.00 | 0 | 0 |
| 1 × MIC | | | | | | | | |
| IS111 | 5.6 ± 0.30 | 0.8 ± 0.13 | 0.4 ± 0.08 | 0 | 0 | 0 | 0 | 0 |
| 5 × MIC | | | | | | | | |
| 1 × MIC | 5.7 ± 0.33 | 0.7 ± 0.15 | 0 | 0 | 0 | 0 | 0 | 0 |
| Ciprofloxacin | | | | | | | | |

Results shown in the table are Mean ± SD obtained from triplicate experiments.

### Conclusion

Peptide IS 111 has showed good antimicrobial activity at the concentration of 1×MIC (3.13 μg/ml) against S. aureus and at 3 hrs, 5×MIC of IS 111 and 1×MIC of ciprofloxacin display similar killing activities.

Time Kill Kinetics of Test Peptide IS 111 Against P. aeruginosa:

The result obtained with the test compound IS 111 and P. aeruginosa was shown in FIG. 20. After 24 h incubation with 0.5×MIC (1.56 μg/ml) of IS 111, a 1 log 10 CFU/ml reduction in viability of P. aeruginosa occurred indicating the IS 111 was bacteriostatic against this strain. At a concentration of 1×MIC (3.13 μg/ml), however, the IS 111 was bacteriostatic against P. aeruginosa by 12 h. Also, increasing the concentration to 5×MIC (15.6 μg/ml) resulted in bacteriostatic activity against P. aeruginosa by 3 h (FIG. 20 & Table 25). Complete Reduction of Initial inoculum of P. aeruginosa was achieved by 3 h treatment with 5×MIC. These results indicate that anti-bacterial activity of IS 111 was both time- and concentration-dependent. In comparison, the killing activity of Ciprofloxacin at 1×MIC was Similar to 5×MIC of IS 111 and Complete Reduction of Initial inoculum of P. aeruginosa was achieved by 3 h treatment. At 3 hrs, the Peptide IS 111 and ciprofloxacin display similar killing activities.

Results shown in the table are Mean±SEM obtained from triplicate experiments where P<0.005, P<0.05 and denoted as *** & ** when compared with vehicle control group respectively.

Time-kill profiles for P. aeruginosa in Trypticase soya agar during treatment with 1.565 μg/ml (0.5×MIC), 3.13 μg/ml (1×MIC) and 15.65 μg/ml (5×MIC) IS 111 and Ciprofloxacin 0.5 μg/ml (1×MIC) as a standard.

TABLE 25
Time Kill Kinetics of test peptide IS 111 against P. Aeruginosa:

| | Viable count at different time of exposure (1 × 10⁵ CFU/ml) | | | | | | | |
|---|---|---|---|---|---|---|---|---|
| Groups | 0 h | 1 h | 2 h | 3 h | 4 h | 5 h | 12 h | 24 h |
| Vehicle | 6.4 ± 0.20 | 5.9 ± 0.23 | 6.2 ± 0.18 | 6.7 ± 0.25 | 7.2 ± 0.20 | 7.3 ± 0.15 | 6.6 ± 0.20 | 6.4 ± 0.18 |
| Control | | | | | | | | |
| IS111 | 6.3 ± 0.30 | 5.4 ± 0.18 | 4.9 ± 0.15 | 4.1 ± 0.20 | 3.5 ± 0.15 | 2.8 ± 0.23 | 2.4 ± 0.15 | 2.1 ± 0.25 |
| 0.5 × MIC | | | | | | | | |
| IS111 | 6.4 ± 0.15 | 4.0 ± 0.30 | 1.9 ± 0.20 | 1.0 ± 0.08 | 0.5 ± 0.08 | 0.2 ± 0.05 | 0.0 | 0.0 |
| 1 × MIC | | | | | | | | |
| IS111 | 6.4 ± 0.15 | 0.4 ± 0.10 | 0.0 | 0.0 | 0.0 | 0.0 | 0.0 | 0.0 |
| 5 × MIC | | | | | | | | |
| 1 × MIC | 6.4 ± 0.25 | 3.1 ± 0.35 | 1.4 ± 0.17 | 0.0 | 0.0 | 0.0 | 0.0 | 0.0 |
| Ciprofloxacin | | | | | | | | |

Results shown in the table are Mean ± SD obtained from triplicate experiments.

### Conclusion

Peptide IS 111 has showed good antimicrobial activity at the concentration of 1×MIC (3.13 μg/ml) against P. aeruginosa and at 3 hrs, 5×MIC of IS 111 and 1×MIC of ciprofloxacin display similar killing activities.

Time Kill Kinetics of Test Peptide IS 111 Against E. coli:

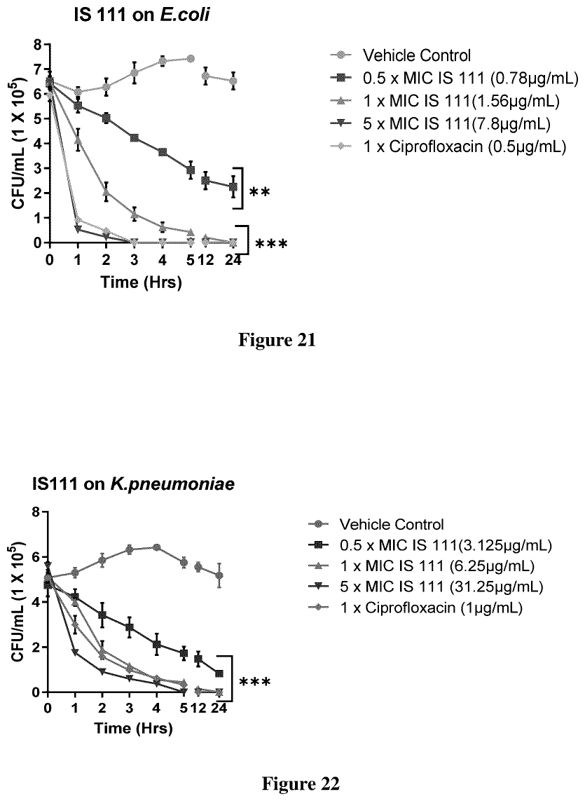

The result obtained with the test compound IS 111 and E. coli was shown in FIG. 21. After 24 h incubation with 0.5×MIC (0.78 μg/ml) and 1×MIC (1.56 μg/ml) of IS 111, a 2 log 10 CFU/ml reduction in viability of E. coli occurred and complete reduction of the inoculum, respectively (FIG. 4) and indicating the IS 111 was bacteriostatic against this strain. Also, increasing the concentration to 5×MIC (7.8 μg/ml) resulted in bacteriostatic activity against E. coli by 3 h (FIG. 21 & Table 26). Complete Reduction of Initial inoculum of E. coli was achieved by 3 h treatment with 5×MIC. These results indicate that anti-bacterial activity of the IS 111 was both time- and concentration-dependent. In comparison, the killing activity of Ciprofloxacin at 1×MIC was Similar to 5×MIC of IS 111 and Complete Reduction of Initial inoculum of E. coli was achieved by 3 h treatment. At 3 h, the Peptide IS 111 and ciprofloxacin display similar killing activities.

FIG. 21 shows the time-kill kinetics of test peptide IS 111 against E. coli. Results shown in the table are Mean±SEM obtained from triplicate experiments where P<0.005, P<0.05 and denoted as *** & ** when compared with vehicle control group respectively. Time-kill profiles for E. coli in Trypticase soya agar during treatment with 0.78 μg/ml (0.5×MIC), 1.565 μg/ml (1×MIC) and 7.8 μg/ml (5×MIC) IS 111 and Ciprofloxacin 0.5 μg/ml (1×MIC) as a standard.

TABLE 26
Time Kill Kinetics of test peptide IS 111 against E. coli:

| | Viable count at different time of exposure (1 × 10⁵ CFU/ml) | | | | | | | |
|---|---|---|---|---|---|---|---|---|
| Groups | 0 h | 1 h | 2 h | 3 h | 4 h | 5 h | 12 h | 24 h |
| Vehicle | 6.5 ± 0.10 | 6.1 ± 0.20 | 6.3 ± 0.35 | 6.9 ± 0.43 | 7.3 ± 0.20 | 7.4 ± 0.10 | 6.7 ± 0.35 | 6.5 ± 0.35 |
| Control | | | | | | | | |
| IS111 | 6.4 ± 0.48 | 5.5 ± 0.28 | 5.0 ± 0.20 | 4.2 ± 0.10 | 3.7 ± 0.08 | 2.9 ± 0.35 | 2.5 ± 0.35 | 2.3 ± 0.43 |
| 0.5 × MIC | | | | | | | | |
| IS111 | 6.5 ± 0.10 | 4.2 ± 0.45 | 2.1 ± 0.38 | 1.2 ± 0.28 | 0.6 ± 0.20 | 0.4 ± 0.10 | 0.2 ± 0.08 | 0 |
| 1 × MIC | | | | | | | | |
| IS111 | 6.5 ± 0.23 | 0.5 ± 0.05 | 0.2 ± 0.05 | 0 | 0 | 0 | 0 | 0 |
| 5 × MIC | | | | | | | | |
| 1 × MIC | 6.0 ± 0.25 | 0.9 ± 0.10 | 0.5 ± 0.05 | 0 | 0 | 0 | 0 | 0 |
| Ciprofloxacin | | | | | | | | |

Results shown in the table are Mean ± SD obtained from triplicate experiments.

### Conclusion

Peptide IS 111 has showed good antimicrobial activity at the concentration of 1×MIC (1.56 μg/ml) against E. coli and at 3 hrs, 5×MIC of IS 111 and 1×MIC of ciprofloxacin display similar killing activities.

Time Kill Kinetics of Test Peptide IS 111 Against K. pneumoniae:

The result obtained with the test compound IS 111 and K. pneumoniae was shown in FIG. 22. After 24 h incubation with 0.5×MIC (3.13 μg/ml) of IS 111, a <1 log 10 CFU/ml reduction in viability of K. pneumoniae occurred, indicating the IS 111 was bacteriostatic against this strain. At a concentration of 1×MIC (6.25 μg/ml), however, the IS 111 was bacteriostatic against K. pneumoniae by 24 h. Also, increasing the concentration to 5×MIC (31.25 μg/ml) resulted in bacteriostatic activity against K. pneumoniae by 5 h (FIG. 22 & Table 27). Complete Reduction of Initial inoculum of K. pneumoniae was achieved by 5 h treatment with 5×MIC. These results indicate that anti-bacterial activity of the IS 111 was both time- and concentration-dependent. In comparison, the killing activity of Ciprofloxacin at 1×MIC was slower and Complete Reduction of Initial inoculum of K. pneumoniae was achieved by 12 h treatment. At 12 h, the Peptide IS 111 and ciprofloxacin display similar killing activities.

FIG. 22 shows time-kill kinetics of test peptide IS 111 against K. pneumoniae. Results shown in the table are Mean±SEM obtained from triplicate experiments where P<0.005, P<0.05 and denoted as *** & ** when compared with vehicle control group respectively. Time-kill profiles for K. pneumoniae in Trypticase soya agar during treatment with 3.125 μg/ml (0.5×MIC), 6.25 μg/ml (1×MIC) and 31.25 μg/ml (5×MIC) IS 111 and Ciprofloxacin 1 μg/Ml (1×MIC) as a standard.

TABLE 27
Time Kill Kinetics of test peptide IS 111 against K. pneumoniae:

| | Viable count at different time of exposure (1 × 10⁵ CFU/ml) | | | | | | | |
|---|---|---|---|---|---|---|---|---|
| Groups | 0 h | 1 h | 2 h | 3 h | 4 h | 5 h | 12 h | 24 h |
| Vehicle | 5.1 ± 0.23 | 5.3 ± 0.23 | 5.9 ± 0.30 | 6.3 ± 0.20 | 6.4 ± 0.10 | 5.8 ± 0.23 | 5.6 ± 0.20 | 5.2 ± 0.53 |
| Control | | | | | | | | |
| IS111 | 4.8 ± 0.50 | 4.2 ± 0.35 | 3.4 ± 0.55 | 2.9 ± 0.45 | 2.1 ± 0.48 | 1.7 ± 0.30 | 1.5 ± 0.33 | 0.8 ± 0.13 |
| 0.5 × MIC | | | | | | | | |
| IS111 | 5.1 ± 0.23 | 4.0 ± 0.18 | 1.9 ± 0.40 | 1.2 ± 0.15 | 0.6 ± 0.03 | 0.5 ± 0.05 | 0.2 ± 0.15 | 0 |
| 1 × MIC | | | | | | | | |
| IS111 | 5.6 ± 0.18 | 1.8 ± 0.13 | 0.9 ± 0.08 | 0.6 ± 0.08 | 0.4 ± 0.05 | 0 | 0 | 0 |
| 5 × MIC | | | | | | | | |
| 1 × MIC | 5.1 ± 0.18 | 3.0 ± 0.40 | 1.6 ± 0.10 | 1.0 ± 0.05 | 0.6 ± 0.08 | 0.3 ± 0.03 | 0 | 0 |
| Ciprofloxacin | | | | | | | | |

Results shown in the table are Mean ± SD obtained from triplicate experiments.

### Conclusion

Peptide IS 111 has showed good antimicrobial activity at the concentration of 1×MIC (6.25 μg/ml) against K. pneumoniae and at 12 hrs, 5×MIC of IS 111 and 1×MIC of ciprofloxacin display similar killing activities.

### Conclusion of Time Kill Assays:

Time-killing assay was performed treatment of organisms with test peptide IS 111 for 24 h and IS 111 has showed good antimicrobial activity at the concentration of 1×MIC (1.56 μg/ml) against S. aureus, 1×MIC (0.78 μg/ml) against P. aeruginosa, and at 2 h, 5×MIC of IS 111 showed the completed reduction of initial inoculum. 5×MIC of IS 111 and 1×MIC of ciprofloxacin display similar killing activities, whereas IS 111 has showed good antimicrobial activity at the concentration of 1×MIC (0.39 μg/ml) against E. coli and at 3 h, 5×MIC of IS 111 and 1×MIC of ciprofloxacin display similar killing activities. IS 111 has showed good antimicrobial activity at the concentration of 1×MIC (1.56 μg/ml) against K. pneumoniae and at 12 h, 5×MIC of IS 111 and 1×MIC of ciprofloxacin display similar killing activities. Qualitative analysis of bacteria survival/time-killing assay, at different time intervals.

It is well known that the incidence of pathogen resistance to antibiotics is increasing day by day and is the most serious issue in today's world [Infectious Diseases Society of America, 2004; Peters N K et al. 2008]. Bacterial pathogens (ethological agents of human illnesses) are a severe hazard to human health and the antibiotic abuse and misuse generate major environmental concerns, as well as the evolution and rapid spread of antibiotic resistance bacteria [Gums J G 2002; Jorgensen and Ferraro, 2009], resulting in an increasing number of deaths each year [Nathan and Cars, 2014].

Most of the current antimicrobials have natural origin deriving from microbes, plants or animals [Bérdy, 2005] like Host defense peptides (HDPs) and/or antimicrobial peptides (AMPs) [Steinstraesser et al., 2009]. Antimicrobial peptides are classified based on their mode of action, which can include interfering with cell wall synthesis, protein, DNA, or RNA synthesis, as well as inhibiting various metabolic pathways or the cell cycle [Hancock and Sahl, 2006; Hale and Hancock, 2007; Hilpert et al., 2010; Maróti and Kondorosi, 2014]. Due to some toxicity profile, the synthetic HDPs are the focused area of research to combat the resistance to the various pathogens.

In the present study, the IS 111, one of the synthetic peptide known as Host Defense Peptide (HDP/AMP) was investigated for its anti-bacterial profile by estimating MIC and time kill survival rates. Our study results showed that IS 111 was significant inhibitory properties against S. aureus (ATCCR 6538™) 1.56 (μg/ml), P. aeruginosa (ATCC® 9027™) 0.78 (μg/ml), E. coli (ATCCR 8739™) 0.39 (μg/ml) and K. pneumoniae (ATCC® 700603™) 1.56 (μg/ml).

Based on the MIC results, investigation was continued regarding time kill survival to determine how long all bacteria are necessary for IS 111 to eliminate above mentioned pathogens. Unlike an MBC/MIC assay, this assay enables the measurement of the compound's rate of cidal action [Aiyegoro, Afolayan, & Okoh, 2009]. The curves were determined to assess the correlation between MIC and bactericidal activity of IS 111 at concentrations ranging from 0.5-fold MIC to 5-fold MIC [Mohamed F. et al., 2016].

The compound was rapidly bactericidal at 1×MIC for all four pathogens after 1 h incubation. Meanwhile S. aureus and P. aeruginosa were completely eliminate after 2 h incubation, whereas E. coli after 3 h incubation and K. pneumoniae after 4 h incubation at concentration of 5×MIC. The 5×MIC concentration of IS 111 are comparable with 1×MIC of ciprofloxacin and display similar killing activities. The results of the time-kill assay are presented in FIGS. 42-46 and the data showed that the response of the bacteria to the tested compound IS 111 varied among the strains, concentration, and time dependent. The differences in susceptibility may be due to the differences in cell wall composition and/or genetic content of their bacteria. The antibacterial activity is most likely due to the adsorption of compounds causing membrane disruption, subsequent leakage of cellular contents and cell death.

Based on literature study, peptide IS 111 is designed antimicrobial host defense peptide (HDP). In general, the anti-microbial activity of AMP's might be as follows, where the AMPs must interact with membranes as part of their direct antibacterial mechanism (or mechanisms) of action, leading to membrane perturbation, disruption of membrane-associated physiological events such as cell wall biosynthesis or cell division, and/or translocation across the membrane to interact with cytoplasmic targets and destroy the cell by changing membrane conductance and altering intracellular function and alterations in membrane structure results in the reorientation of peptide molecules in the membrane culminating in eventual pore formation and lysis of the target microbe. The concentrations of the peptides also play an important role which promotes the cell lysis, and capability of channel formation [Jaynes, J. M. Drug News & Perspectives 3:69 [1990]; and Reed, W. A. et al. Molecular Reproduction and Development 31:106 1992].

Thus, at certain concentrations, these peptides stimulate or create channels that can be advantageous to the normal mammalian cell in a benign environment where it is not necessary to exclude toxic chemical compounds. The test compound at a concentration equal to 5×MIC was rapidly bactericidal, achieving complete elimination of both test bacterial strains within 3 h. All the time-kill data obtained with the test compound IS 111 showed its antibacterial activity to be time- and concentration-dependent. In follow-up studies, the test peptide will be examined to see if the in vitro time-kill statistics are predictive of in vivo efficacy.

IS 111 has showed good antimicrobial activity, almost like standard antibiotic Ciprofloxacin. Thus, the anti-microbial effect of IS 111 would suggest the possible utilization of synthetic peptides as effective anti-bacterial agents against pathogenic bacteria.

### Example 4: Confirmatory Studies for In Vitro Activity of Test Peptide IS 111 on BALB/C Mice Peritoneal Macrophages Stimulated with LPS

### Effect of IS 111 on Viability of BALB/C Mice Peritoneal Macrophages:

The viability and cytotoxicity of different concentrations of IS 111 to peritoneal macrophages were examined by MTT assay. The peritoneal macrophages were incubated with IS 111 in different concentrations ranging from (0.5 to 100 μg/mL) and cell viability was measured by an MTT assay 18 h later. It was found that IS 111 from 1.56 to 100 μg/mL had no cytotoxic effects on murine peritoneal macrophages. These results confirmed that the effects of IS 111 on murine peritoneal macrophages were not due to a reduction in cell viability. Therefore, subsequent assays were carried out at concentrations less than 100 μg/mL.

### Effect of IS 111 on the Levels of Cytokines in LPS-Stimulated Peritoneal Macrophages:

### Immunomodulatory Activity.

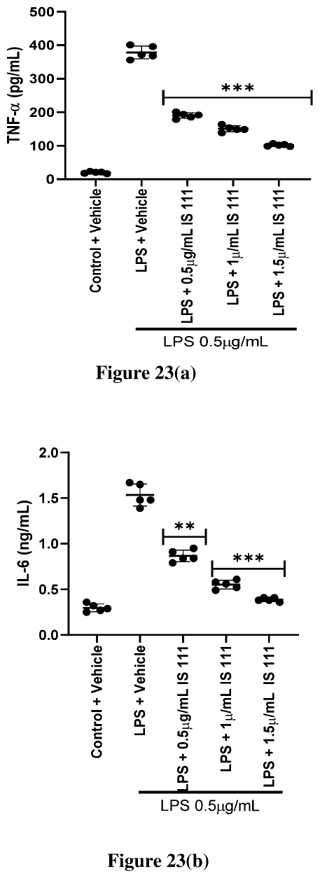

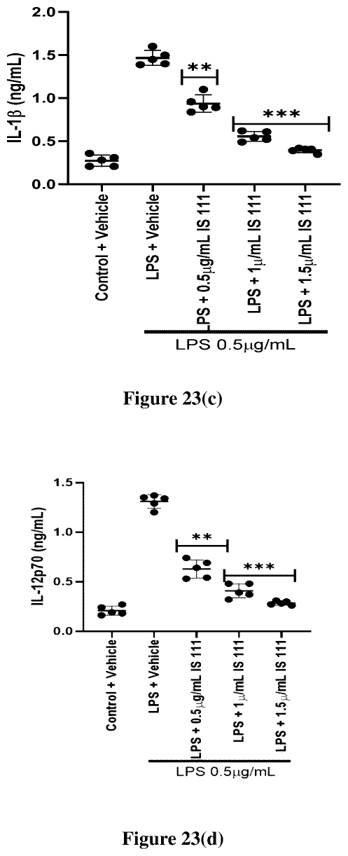

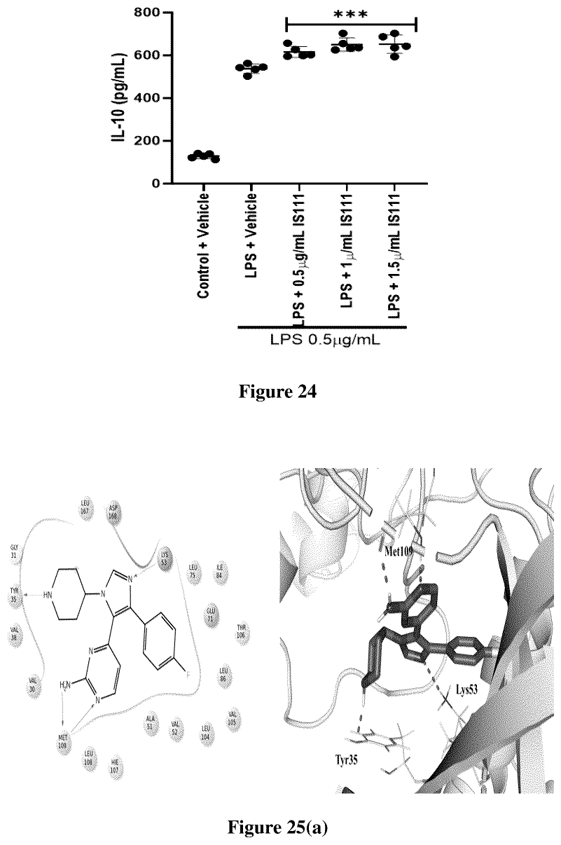

IS 111 inhibits LPS-induced macrophage activation and the inflammatory response. Macrophages are known to produce all kinds of inflammatory factors to be involved in the progression of sepsis. When stimulated with LPS, the number of mouse peritoneal macrophages increases, and an excessive immune response is triggered. Cell viability assay was used to evaluate the cytotoxicity of IS 111 on peritoneal macrophages; the result indicated that the concentrations less than 100 μg/mL had no obvious cytotoxicity. In the subsequent experiments, concentrations of IS 111 did not exceed 100 μg/mL. To further confirm the anti-inflammatory effects of IS 111 on macrophages, the peritoneal macrophages from mice were collected to measure the production of IL-6, IL-1β, IL-12P70 and TNF-α and the same inhibitory effects were observed (FIG. 23) and IL-10 levels. (FIG. 24).

TNF-α and IL-6 are known to be a pro-inflammatory mediator in inflammatory diseases. In an attempt to determine whether IS 111 regulates TNF-α and IL-6 production, peritoneal macrophages at a range of 0.8-1×10⁶ were treated with IS 111 (0.5, 1.0 & 1.5 μg/mL) in the presence or absence of LPS stimulation. As shown in FIG. 48, LPS challenge markedly increased the levels of pro-inflammatory cytokines IL-6, IL-1β, IL-12P40 and TNF-α in peritoneal macrophages as compared with those in the normal control group (P<0.001), Following pre-treatment with IS111, obviously reduced LPS-induced production of IL-6, IL-1β, IL-12P40 and TNF-α levels were significantly decreased in a dose-dependent manner. This confirmed the anti-inflammatory effect of IS 27 on secretion of IL-10 Levels (FIG. 26) also along with the pro inflammatory cytokines, IL-6, IL-1β, IL-12P40 and TNF-α levels in LPS-stimulated murine peritoneal macrophages.

The expression of different cytokines estimated in peptide IS 111 treated RAW 264.7 cells by IL-1β, IL-6, IL-12p70, TNF-α and IL-10 ELISA assay. The results showed that the cytokine production levels of IL-1β, IL-6, IL-12p70 and ITNF-α were significantly inhibited by peptide IS 111 treatments in LPS-stimulated RAW 264.7 cells. Compared to IL-6, production TNF-α has been reduced to a greater extent. Maximum inhibition for all cytokines was observed at 100 μM.

FIG. 23 represents TNF-α (a), IL-6 (b) IL-1β (c) and IL-12p70 (d) cytokines level detection in vitro mice macrophages. FIG. 24 shows IL-10 cytokines level detection in vitro mice macrophages. The values represent the means of at least three independent experiments performed in triplicate (mean±SEM). A significant difference from the control (LPS alone) was indicated as P<0.001, P<0.01 and denoted as **** & *** respectively.

RAW 264.7 cells were pretreated with peptide IS 111 (0.5, 1 & 1.5 μg/ml) for 1 h, followed by 0.5 μg/mL LPS stimulation for 24 h and evaluated for IL-10. ELISA was used to measure the production of cytokines. Triplicate experiments were conducted and repeated for three times. The values represent the means of at least three independent experiments performed in triplicate (mean±SEM). A significant difference from the control (LPS alone) was indicated as P<0.001, P<0.01 and denoted as **** & *** respectively.

IL-6, TNF-α, IFN-γ and IL-1β are pro-inflammatory cytokines that modulate immunity and inflammation. Peptide IS 111 has anti-inflammatory effects in LPS-induced RAW 264.7 cells, including suppression of the underlying molecular mechanism of IL-6, TNF-α, IL-12 and

IL-1β. When stimulated with LPS, macrophages undergo a series of changes that ultimately lead to cytokine secretion. Peptide IS 111 inhibitory effects on IL-6, TNF-α, IL-12, and IL-1β secretion has been observed. Our observations from the study concluded the Peptide IS 111 showed immune-suppressive activity at different concentrations. It can, therefore, be inferred that peptide IS 111 has therapeutic potential and could act as an active immunomodulatory candidate without any adverse effects, supporting peptide IS 111 conventional arguments for medicinal purposes.

### Example 5: Acute Toxicity Study for all Peptide IS 111 in BALB/C Mice

The study was planned according to the following OECD Guidelines (The FDP was adopted as an OECD Guideline (OECD 420) in 1992 but as an alternative for OECD 401, not a replacement. In 1996, a second alternative method, the Acute Toxic Class Method (ATC) was adopted (OECD 423) and this was followed in 1998 by the Up and Down Procedure (UDP; OECD 425)).

The safety of the peptide was tested on mice and rats at the age of 6-8 weeks. The acute toxicity of the peptide IS 111 was conducted with five males and females of mice and rats per each group.

BALB/C mice (5 animals/sex/group) were administered with the peptides IS 111 at different dose levels i.e., low (0.6 mg/kg), mid (2.4 mg/kg) and high (4.8 mg/kg) with single dose administration via subcutaneous route. The control animals were administered with 0.9% Nacl solution at the dose volume of 10 ml/kg b. wt. The functional observations were carried out for every 2 hrs till 24 hrs continuously after exposure to the peptide for 14 days of the study. No mortality of the animals was seen at the point of observation till on 14th day. Thus, the peptide IS111 does not show any acute lethality and all the animals are active on 14th day, when administered once.

### Example 6: Molecular Docking Studies of IS 111

Small peptide molecules were allowed to dock within the grid by standard precision (SP) docking. The Peptide IS 111 observed to show significant glide score in SP docking. Docking scores of the co-crystal ligand and the small peptide was shown in respective tables and the molecular interaction of peptide was shown in their respective figures.

### Molecular Docking Studies of Peptide IS 111 Against Map Kinase

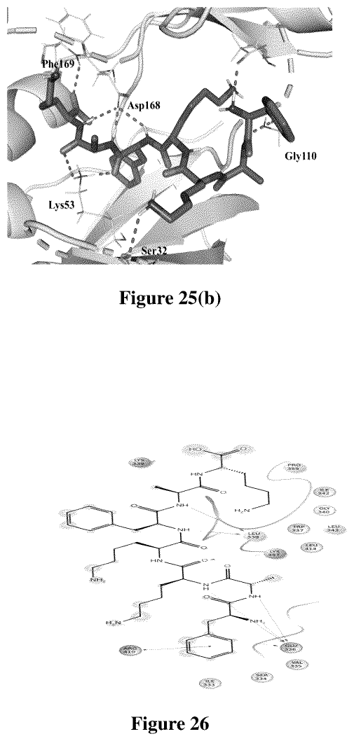

The crystal structure of Map kinase (1BL7) in complex with 4-(4-fluorophenyl)-1-(4-piperidinyl)-5-(2-amino-4-pyrimidinyl)-imidazole was explored to find residues such as Met109, Tyr35, Lys 53 were forming hydrogen bonds with inhibitor and residues such as Gly31, Val38, Val30, Leu108, Ala51, Val52, Leu86, Glu71, Leu75, Asp168 were present within the 4 Å region of Co-crystal ligand. This site was used to generate grid for molecular docking studies (FIG. 25a). According to the docking scores of the peptide in comparison with the co-crystal ligand, IS-111 is showing the better binding affinity (Table 28). The binding orientation of IS-111 is similar to the co-ligand. Both the terminals of the IS-111 are forming direct hydrogen bonding with 5 residues of the protein. Interestingly it is forming hydrogen bond with one of the key residues Lys53 in comparison with co-crystal ligand. Interactions were represented in the FIG. 25b.

TABLE 28
Docking scores of the peptide IS 111 along with co-crystal (1BL7):

| S. No | Compound Code | Docking Score (kcal/mol) |
|---|---|---|
| 1 | Co-crystal ligand | −9.010 |
| 2 | IS-111 | −9.098 |

FIG. 25(a) represents molecular docking interactions of test peptide IS 111 with the receptor Map kinase. Active pocket of Map kinase in complex with 4-(4-fluorophenyl)-1-(4-piperidinyl)-5-(2-amino-4-pyrimidinyl)-imidazole. The color code representations for the picture as yellow: protein; magenta: co-crystal ligand. The amino acids represented in lines and co-ligand was in sticks. FIG. 25(b) represents molecular docking interactions of peptide IS 111 with the receptor Map kinase (1BL7).

### Molecular Docking Studies of Peptide IS 111 Against TNF-Alpha

The crystal structure of TNF-alpha (4K8U) was retrieved form PDB. Active site was determined using sitemap module of Schrodinger software. The best scoring site was used to generate grid for molecular docking studies. According to the docking scores in comparison with the co-crystal ligand, IS-111 is showing the better binding affinity. IS-111 is forming hydrogen bonding with seven residues of the protein. Interactions were represented in FIG. 26. FIG. 26 shows molecular docking interactions of peptide IS 111 with the receptor TNF-alpha (4K8U).

TABLE 29
Docking scores of the peptide IS 111
along with co-crystal ligand (4K8U)

| S. No | Compound Code | Docking Score (kcal/mol) |
|---|---|---|
| 1 | IS-111 | −9.789 |

### Molecular Docking Studies of Peptide IS 111 Against VEGF1

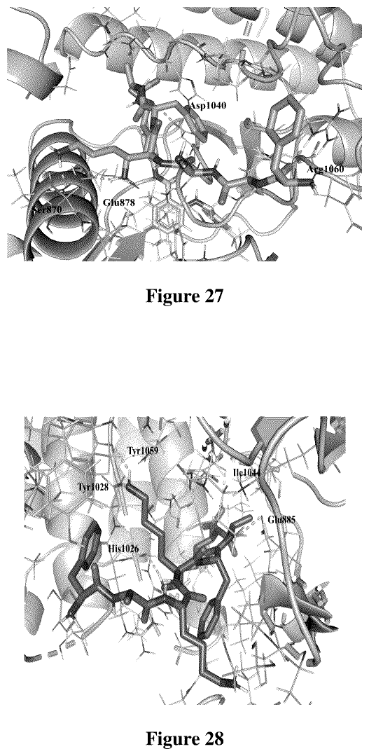

The crystal structure of VEGF1 (3HNG) was retrieved form PDB. Active site was determined using sitemap module of Schrodinger software. The best scoring site was used to generate grid for molecular docking studies. According to the docking scores in comparison with the co-crystal ligand, IS-111 is showing the better binding affinity. IS-111 is forming hydrogen bonding with 4 residues of the protein. Interactions were represented in the FIG. 27. FIG. 27 shows molecular docking interactions of peptide IS 111 with the receptor VEGF1 (3HNG).

TABLE 30
Docking scores of the peptide IS 111
along with co-crystal ligand (3HNG):

| S. No | Compound Code | Docking Score (kcal/mol) |
|---|---|---|
| 1 | IS-111 | −7.520 |

### Molecular Docking Studies of Peptide IS 111 Against VEGF-2

The crystal structure of VGEF-2 (3VHE) was retrieved form PDB. Active site was determined using sitemap module of Schrodinger software. The best scoring site was used to generate grid for molecular docking studies. According to the docking scores in comparison with the co-crystal ligand, IS-111 is showing the better binding affinity. IS-111 is forming hydrogen bonding with four residues of the protein. Interactions were represented in the FIG. 28. This figure represents molecular docking interactions of peptide IS 111 with the receptor VGEF-2 (3VHE).

TABLE 31
Docking scores of the peptide IS 111
along with co-crystal ligand (3VHE):

| S. No | Compound Code | Docking Score (kcal/mol) |
|---|---|---|
| 1 | IS-111 | −7.489 |

### Molecular Docking Studies of Peptide IS 111 Against VEGF-3

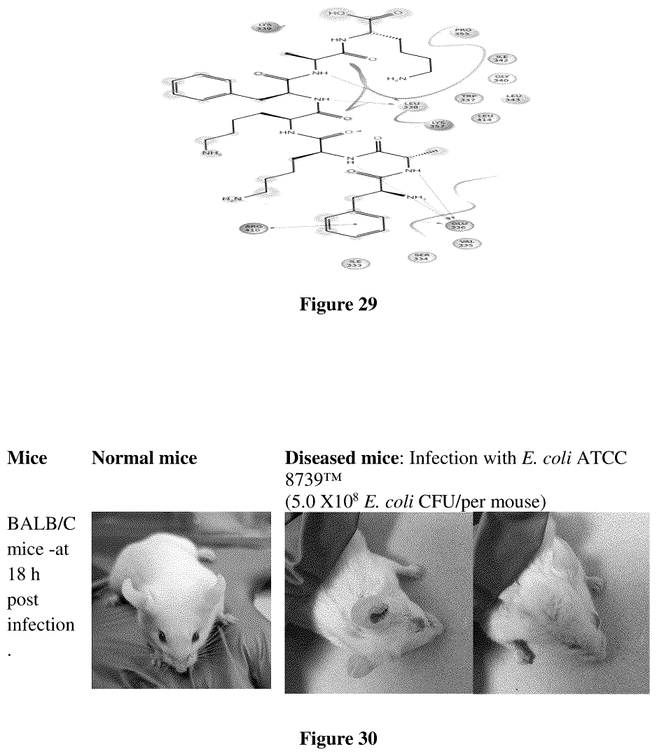

The crystal structure of VGEF-3 (4BSJ) was retrieved form PDB. Active site was determined using sitemap module of Schrodinger software. The best scoring site was used to generate grid for molecular docking studies. According to the docking scores in comparison with the co-crystal ligand, IS-111 is showing the better binding affinity. IS-111 is forming hydrogen bonding with four residues of the protein. Interactions were represented in the FIG. 29. This figure Molecular docking interactions of peptide IS111 with the receptor VGEF-3 (4BSJ).

TABLE 32
Docking scores of the peptide IS 111
along with co-crystal ligand(4BSJ)

| S. No | Compound Code | Docking Score (kcal/mol) |
|---|---|---|
| 1 | IS-111 | −6.372 |

The in silico data explains the mechanism of Peptide IS 111 that mediate these actions of MAP kinases during the response to TNFα. In general, at the MAP kinase level, VEGF mainly activates ERK1/2 and p38 MAP kinases in human endothelial cells. TNF-alpha is able to activate all three MAP kinase cascades as well as the classical inflammatory pathway. Furthermore, the MEK/ERK module of MAP kinases appears to act as the convergence point of VEGF- and TNF-alpha-initiated signaling cascades. This MAP kinase signaling pathways induce a secondary response by increasing the expression of several inflammatory cytokines (including TNFα) that contribute to the biological activity of TNFα. MAP kinases therefore function both upstream and down-stream of signaling by TNFα receptors.

### Example 7: Confirmatory Studies: In Vivo Efficacy Activity of Test Peptide IS 111 Using E. coli Induced Peritonitis Animal Model & Cecal Ligation Puncture (CLP) Animal Model

Results of In Vivo Efficacy Activity of Test Peptide IS 111 Using E. coli Induced Peritonitis Animal Model
Synthetic Peptide IS 111 Provided Broad-Spectrum Protection Against Lethal Infections Caused by E. coli in Mice.

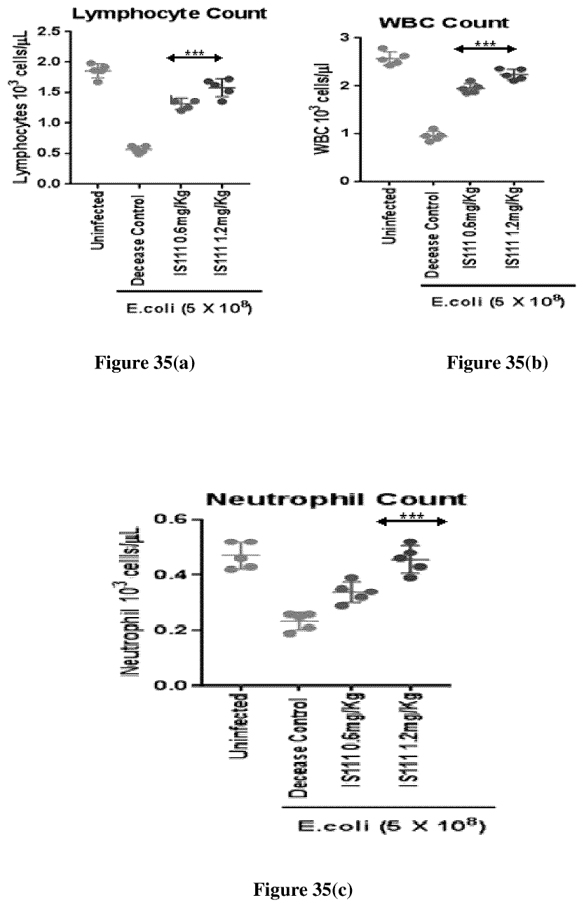

Due to the potent and very promising antimicrobial properties of IS 111, it was decided to test its immunomodulatory activity. In the present study, an aggressive bacterial infection mouse model of bacterial infection was established with E. coli ATCC 8739™ bacteria via intraperitoneal (IP) injection, and 2 hours later treated IV with 0.6 & 1.2 mg/kg of IS 111 peptide suspended in sterile saline. The in vivo protective activity of IS111 was also evaluated by using an invasive infection in the E. coli model. All treatments were performed daily for 5 days. After 18 h of infection, half of animals from each group are anaesthetized for blood and euthanized for organ collection and other subset will continue to be observed for survival rate for 5 days. Mice treated with IS 111 appear clinically healthier after induction of septic shock, when compared to infected animals. BALB/c mice were given either normal saline or 0.6 mg/kg & 1.2 mg/kg of IS 111 IV after 2 h of infection with 5.0×10⁸ E. coli CFU. 18 h later parameters listed in table 80 were observed. Each group consisted of 5-6 mice and photographic representation of mice are shown in the figure. 35. This also confirms the development of E. coli infection with 5.0×10⁸ E. coli CFU and the mice were graded according to the severity of the symptom: normal, mild, marked, and severe. Mice treated with IS 111 appear clinically healthier after induction of septic shock.

TABLE 33
Activity index of mice after 18 hr of induction of septic shock:

| | Disease Control | | |
|---|---|---|---|
| Parameter | (E. coli Infection) | IS 111-0.6 mg/kg | IS 111 -1.2 mg/kg |
| Conjunctivitis | Extreme (Both eyes | Mild (Usually serous | Mild (both eyes are |
| | closed with heavy serous | discharge present in one | open with moderate |
| | discharge) (severe) | eye, eyes were open) | discharge) |
| Hair coat | Extremely ruffled coat, | Moderately ruffled coats | Mild ruffling in most |
| | ruffling evident as early | at 20 h. No ruffling seen | animals. Some animals |
| | as 3 h and at 18 h. | at 3 h. | had shiny unruffled |
| | | | coats. |
| Activity upon | Moribund, extremely | Sluggish activity. Animals | Animals remained |
| stimulation | sluggish, animals | exhibited huddling | normally active. |
| | exhibited huddling | behavior but upon | |
| | behavior, did not respond | stimulation responded and | |
| | or move to stimulation. | broke out of huddle. | |

FIG. 30 shows that experimental mice show the signs of infection at 18 h after post E. coli ATCC 8739™ (5.0×10⁸ E. coli CFU/per mouse).

### Experiment 1 Survival Study:

IS 111 Treatment Enhanced Survival of Mice after Induction of Sepsis:

The Kaplan-Meier curve for survival analysis of mice subjected to polymicrobial sepsis and treated with test peptide IS 111 (0.6 & 1.2 mg/kg) showed lower mortality after sepsis when compared with non-treated septic mice. Peritonitis induced by E. coli infection results in 100% lethality at day 8 Induction of infection was described in the methods section and animals were Intravenously (IV) injected with various doses of IS 111 or vehicle at 2 h after post infection and then mice were observed for 18 h (FIG. 33 a) and subset of animals are continued for observation for 5 days (FIG. 33 b). No significant difference was observed when IS 111 was injected at the time of injury (time zero). Notably, treatment with IS 111, 18 h decreased survival, compared with NS-treated mice.

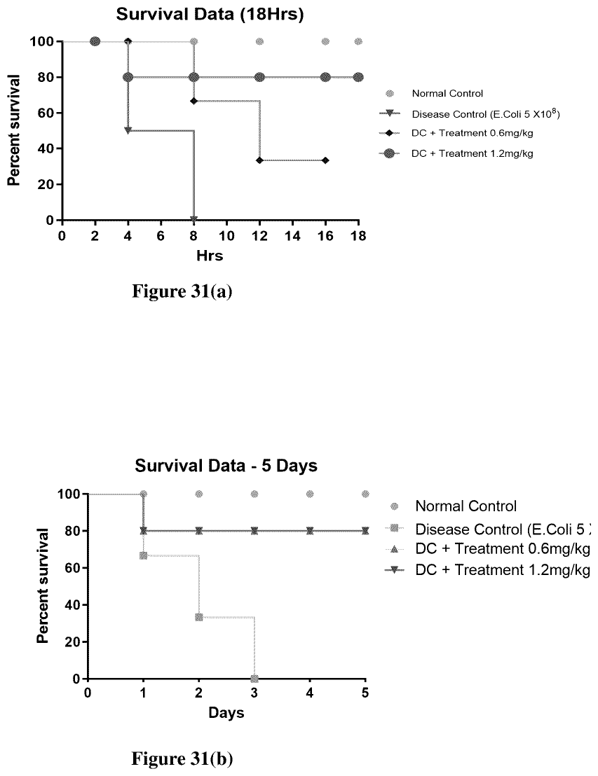

FIG. 31(a) shows that the short synthetic peptide IS 111 increases survival after 18 h of the treatment—after Sepsis Induction. FIG. 31(b) shows the short synthetic peptide IS 111 increases survival after 5 days of the treatment-after Sepsis Induction.

In FIGS. 31(a) and (b) Kaplan-Meier curve for survival analysis of mice subjected to polymicrobial sepsis and treated with peptide SEQ ID No. 1 (IS 111). Data are shown as mean±SD of six mice in each group, analyzed by one-way ANOVA and Tukey post hoc tests. *p<0.05 in relation to sham+saline group and #p<0.05 in relation to E. coli induced lethal infection.

### Experiment 2: Acute Hyper Inflammation Study: Cytokine Estimation in Serum:

### Peptide IS 111 Administered Therapeutically Reduces Levels of Proinflammatory Cytokines, and Improves Survival of Septic Mice.

In this study, however, to have potential as a tool for treatment of sepsis, it is important to study if Peptide IS 111 can reduce inflammation and DIC when administered therapeutically. This helped in unraveling its anti-inflammatory properties, its ability to inhibit DIC in infected mice. For this, Peptide IS 111 was given 1 h post induction of sepsis and it was found that Peptide IS 111 could significantly reduce levels of IL-1b, IL-6, IL-12 p70, and TNF-α in mice infected with 5×10⁸ CFU/ml of E. coli ATCC 8739, indicating that Peptide IS 111 is a potent inhibitor of proinflammatory cytokines when administered therapeutically and anti-inflammatory cytokines, such as IL-10. Polymicrobial sepsis induced by E. coli Infection increased levels of TNF-α & IL-6 in the peritoneal lavage, which was significantly, reduced in mice that received peptide IS 111 therapy (FIG. 32).

FIGS. 32(a) to (d) represent the detection of cytokines (IL-1β, IL-6, IL-12 & TNF-α) in the serum sample of animals after E. coli infection, induction of sepsis and short synthetic peptide IS 111 treatment, after 18 h of polymicrobial sepsis.

Mice (n=5-6/group) and cytokine levels were estimated at 18 h post-E. coli infection via IV route and Data are expressed as mean±SEM. P<0.001, P<0.01 & p<0.05 was considered as statistically significant and denoted as ***, ** & * compared to disease control group.

Levels of proinflammatory cytokines IL-1β, IL-6, IL-12, TNF-α in the serum (FIGS. 34 a, b, c & d), in serum samples at 18 h after induction of polymicrobial sepsis and treatment with synthetic peptide IS 111 at dose of (0.6 & 1.2 mg/kg). Data are shown as mean±SD of six mice in each group, analyzed by one-way ANOVA and Tukey post hoc tests. *p<0.05 in relation to sham+saline group and #p<0.05 in relation to E. coli Induced lethal infection.

FIG. 33 represents detection of cytokines (IL-10) in the serum sample of animals after E. coli Infection, induction of sepsis and short synthetic peptide IS 111 treatment, after 18 h of polymicrobial sepsis.

Mice (n=5-6/group) and cytokine levels were estimated at 18 h post-E. coli infection via IV route and Data are expressed as mean±SEM. P<0.001, P<0.01 & p<0.05 was considered as statistically significant and denoted as ***, ** & * compared to disease control group.

Levels of anti-inflammatory cytokine IL-10 (FIG. 35) in serum samples at 18 h after induction of polymicrobial sepsis and treatment with synthetic peptide IS 111 at dose of (0.6 & 1.2 mg/kg). Data are shown as mean±SD of six mice in each group, analyzed by one-way ANOVA and Tukey post hoc tests. *p<0.05 in relation to sham+saline group and #p<0.05 in relation to E. coli Induced lethal infection.

### Experiment 3: Late Immune Paralysis Studies:

### IS 111 Promotes a Decrease in Pro Inflammatory Cytokine Levels in Peritoneal Lavage Fluid:

The intra-abdominal injury/ischemia, especially with subsequent infection, induces an excessive inflammatory/protein mediator production and uncontrolled inflammation in the peritoneum mostly via the lymphatic pathway) into the systemic circulation may precipitate the deleterious effects of sepsis and multiple organ dysfunction [Stefan Wirtz et al., 2006; Yoon Ju Chol, et al., 2011]. In the present study, the cytokines, bacterial count, and total no of cells are measured in PLF. The amount of secreted cytokines levels in the abdominal lavage fluid was expressed as pg protein/mL.

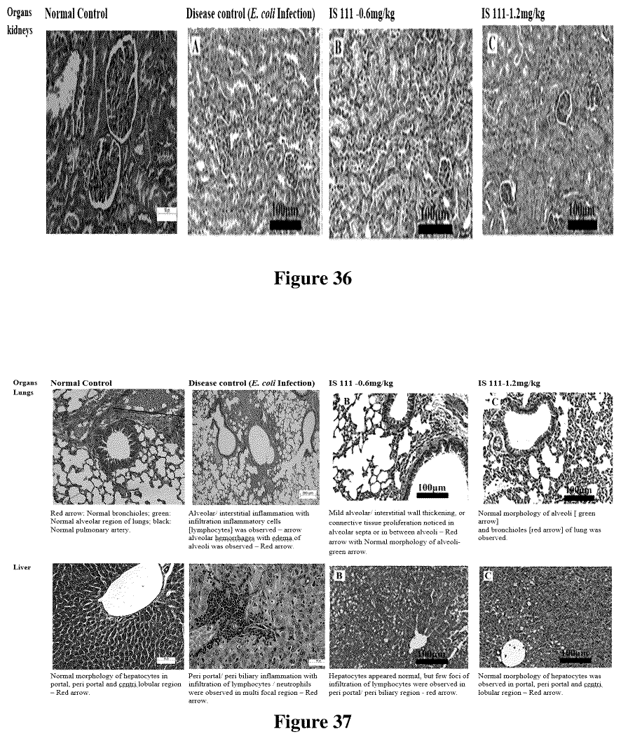

Proinflammatory cytokines (IL-6, and TNF-α), were detected in peritoneal lavage (FIG. 36) from the E. coli infected group. The IS111 treatment by both IV route at 0.6 & 1.2 mg/kg doses significantly reduced the levels of all these mediators, (FIG. 36). Levels of proinflammatory cytokines IL-6 & TNF-α in the peritoneal lavage fluid (a & b), at 18 h after induction of polymicrobial sepsis and treatment with synthetic peptide IS 111 at dose of (0.6 & 1.2 mg/kg). Data are shown as mean±SD of six mice in each group, analyzed by one-way ANOVA and Tukey post hoc tests. *p<0.05 in relation to sham+saline group and #p<0.05 in relation to E. coli Induced lethal infection.

FIGS. 34(a) and (b) represents detection of cytokines (IL-6 & TNF-α) in the Peritoneal lavage fluid sample of animals after E. coli Infection, induction of sepsis and short synthetic peptide IS111 treatment, after 18 h of polymicrobial sepsis.

Mice (n=5-6/group) and cytokine levels were estimated at 18 h post-E. coli infection via IV route and Data are expressed as mean #SEM. P<0.001, P<0.01 & p<0.05 was considered as statistically significant and denoted as ***, ** & * compared to disease control group.

Administration of Peptide IS 111 therapeutically helped to increases Lymphocytes, WBC & Neutrophils in mice infected with E. coli in comparison to 0.9% Nacl treated septic mice and considering the Peptide IS 111 can be act as a first line of defense immune activity. E. coli Infection also resulted in dramatic reduction in numbers of lymphocytes and neutrophils. This reduction in numbers was reflected in the decrease in WBCs post E. coli Infection induced sepsis. Administration of Peptide IS 111 restored lymphocyte, and WBC (FIG. 37) numbers comparable to control mice. CLP also resulted in reduction in blood neutrophil levels. However, neutrophil numbers were not significantly but only marginally higher in Peptide IS 111 treated mice. Thus, Peptide IS 111 is able to prevent inflammation in septic mice, even when administered therapeutically. Because Peptide IS 111 reduced induction of both proinflammatory cytokines in mice infected with a higher dose of E. coli when administered therapeutically, its effects on survival of these mice were next investigated. Mice which were not administered Peptide IS 111 died by 16 h post induction of sepsis. The survival analysis subjected to polymicrobial sepsis after 18 hrs of treatment and after 5 days of treatment with Peptide IS 111. However, therapeutic administration of Peptide IS 111 at 1, 24, and 72 h post injection of E. coli ATCC 8739 dramatically increased survival to 80% as observed at the last time point of 8 h. Thus, Peptide IS 111 improves survival when administered therapeutically to septic mice.

Levels of Lymphocytes, WBC & Neutrophils in the serum (a, b & c), in serum samples at 18 h after induction of polymicrobial sepsis and treatment with synthetic peptide IS 111 at dose of (0.6 & 1.2 mg/kg).

FIGS. 35(a) to (c) represents detection of Lymphocytes, WBC & Neutrophils counts in the serum sample of animals after E. coli Infection, induction of sepsis and short synthetic peptide IS 111 treatment, after 18 h of polymicrobial sepsis.

Mice (n=5-6/group) and cytokine levels were estimated at 18 h post-E. coli infection via IV route and Data are expressed as mean±SEM. P<0.001, P<0.01 & p<0.05 was considered as statistically significant and denoted as ***, ** & * compared to disease control group.

### Histopathological Changes of Lung, Kidney & Liver Tissues:

### IS111 Treatment Restores Organ Damage Following Polymicrobial Infection:

The high bacterial load or virulence can cause an exaggerated inflammatory response, resulting in tissue damage and organ dysfunction, which is mainly seen in sepsis. Organ damage is a leading cause of death in patients with sepsis. Thus, whether the organ protection afforded by IS 111 in E. coli induced infection was investigated. No significant changes were observed in the body weight of the animals and in relation to the organs weight, besides, no macro- or microscopic alteration was detected in the brain, heart, lungs, liver, kidney, and spleen.

The disease control animals infected with E. coli (ATCC 8739™ (5×10⁸ CFU/per mouse) were lethargic and presented histopathological changes characteristic of inflammation in the lungs and liver as compared with the control and IS 111-treated groups, with the presence of edema, hemorrhage, and cellular infiltrate, but the presence of necrosis was not evidence. However, it was possible to observe a moderate reduction in the hemorrhagic process, as well as a reduction in the inflammatory infiltrate in animals infected and treated with IS 111 in relation to the non-treated infected group (FIG. 37). The histological damage and clinical signs were rarely observed in treatment groups. The presence of congestion in the groups also analyzed if any euthanasia procedure causes any congestion. Necrosis was not observed in any analyzed organs; however, the presence of mild periportal edema was detected in the liver of most animals in the non-treated infected group. In summary, the IS 111 treatment was able to reduce tissue damage, especially in the lungs, contributing to animal survival. In addition, compared with the blank control group, the renal tissue of the model group did not show obvious substantial lesions, only the changes of renal function were observed. Compared with the model group, a small part of the renal tubules had proteinuria in the kidney tissue section and rare tubeuria occurred after IS 111 administrations (FIG. 36). Normal morphology of glomerulus [red arrow] and tubules [green arrow] was observed in cortex region in control animals.

To test whether IS 111 treatment restored organ damage against sepsis-induced injury, tissues from vital organs that easily succumb to infection such as the lungs, kidneys, and liver, from all experimental groups to study histopathological changes were harvested. All the tissues from different experimental groups were harvested after 18 h, considering the early phase of immunosuppression and most of the animals of the E. coli infection (disease control) group are not survived while the other treated groups (IS 111 −0.6 mg/kg and IS 111 −1.2 mg/kg groups) lived longer. The stained tissue sections were evaluated under a light microscope (Eclipse E200-LED; Nikon, Kawasaki, Japan) at ×200 magnification.

IS 111 treatments inhibiting TNF-α, IL-6 & IL-1β and organ damage in vivo in septic mice. BALB/c mice were given either 0.6 mg/1.2 mg/kg of IS 111 via IV injection after 2 h of post infection with 5×10⁸ CFU/E. coli ATCC 8739™ via the IP route. The uninfected control group received an equivalent volume of normal saline alone. TNF-α, IL-6 & IL-1β levels in sera were measured by ELISA at 18 h and 7 d post infection. Also, after 18 h mice were sacrificed and observed for the histopathological changes indicated lung and liver tissue in E. coli infected group. Furthermore, it was evident that IS 111 (1.2 mg/kg) reduced these injuries by reducing edema and macrophage infiltration and showed that minimized E. coli infection induced lung & liver damage. In addition, IS 111 (0.6 mg/kg) reduces the injuries to moderate extent.

Tissues from the E. coli infected group showed interstitial edema, infiltration of polymorphnuclear leukocytes and monocytes, hemorrhage, vascular congestion, and cellular in the lungs and liver. Tissue damage was more prominent in the disease control group (FIG. 36 & 37); treatment with IS 111 −1.2 mg/kg alone reversed these changes in all organs studied, (FIG. 36 & 37) towards a normal phenotype to resemble the normal control group; there was no trace of hemorrhage. Hence, the present study provides evidence for the anti-bacterial and anti-inflammatory effects of IS 111 in E. coli-induced sepsis.

FIG. 36 shows photographs of representative sections of kidneys sections were prepared and stained with H&E. visualized at 200× magnification are shown. Data shown in mean±SEM from 3-4 mice of all groups of E. coli induced sepsis, treatments, and control animals-IV route-18 h.

FIG. 37 shows photographs of representative sections of liver (a) and lungs (b) sections were prepared and stained with H&E. visualized at 200× magnification are shown. Data shown in mean±SEM from 3-4 mice of all groups of E. coli induced sepsis, treatments, and control animals-IV route-18 h.

Due to the development of resistant strains of bacteria and in addition to inducing resistance, several antibiotics have lost their effectiveness. Therefore, there is a need to develop alternative antimicrobial drugs for the treatment of infectious diseases [Dellinger R P, 2013; Du B, et al., 2002; Gasnik L B, et al. 2007].

In this study, the aim was to evaluate the activity of IS 111 in the treatment of animals with sepsis induced by extraintestinal pathogenic E. coli. Initially, the E. coli ATCC 8739™ was incubated with IS 111 to evaluate the minimum inhibitory concentration and to test the efficacy of peptide in animals, septicemia was induced in mice by injection of very high doses E. coli ATCC 8739™ (E. coli: 5×10⁸ CFU/per mouse) of Gram-negative E. coli via IP. Such high doses of bacteria were potent inducers of proinflammatory cytokines (TNF-a, IL-1b, and IL-6) and organ damage, which are hallmarks of septicemia. Also, apart from inducing a cytokine storm, high doses of E. coli ATCC 8739™ resulted in 100% fatality by 72 h. Therefore, this is a robust model to study excessive inflammation and septicemia as has been reported previously [Burgelman, M et al., 2021].

In this system the effect of peptide IS 111, which belongs to HDP, a designed anti-microbial peptide which comprises several immunomodulatory proteins [Jesse M. Jaynes et. al., 2012] was tested. The animals are injected with E. coli ATCC 8739™ (E. coli: 5×10⁸ CFU/per mouse), and the animal survival was monitored for at 18 h and a subset of mice for seven days, and a subset of mice were euthanized after 18 h to evaluate immunological, biochemical, and histological parameters, as well as the presence of bacteria in the peritoneal fluid.

The inoculation of bacterium caused the death of 90-100% of the animals within 18 h after infection. On the other hand, IS 111 (0.6 & 1.2 mg/kg) was able to keep 50 & 80% of mice alive after 18 h infection in IS 111−0.6 & 1.2 mg/kg respectively and even after 7 days of infection there was 80% survival of mice alive in IS 111 treatment groups.

It was found that IS 111 significantly reduces levels of TNF-α, IL-1b, and IL-6. Both IL-1b and IL-6 have been shown to be elevated during septicemia [Matsukawa, A., 2003] with a single dose of IS 111—after 2-hour infection attenuates E. coli-induced inflammatory cytokine expression and lethality. However, the role of IL-6 in experimental sepsis models is controversial as IL-6 has both anti- and proinflammatory properties [Wang J et al., 2006; Bin Li a, et al., 2008]. Blockade of IL-6 has been shown to be beneficial in sepsis as well as other inflammatory diseases [Riedemann, N. C., T et al., 2003; Ishimoto, T. et al., 2010] indicating a positive correlation between elevated IL-6 levels and sepsis severity [Stefan Wirtz et al., 2006] This was observed in BALF of infected mice and IS 111 treated groups [Gustavo Matute-Bello et al., 2011; Yung-Yang Liu et al., 2008; Yali Zhang, ang et al. 2015 & Kengo Tomita & Yuna Saito et. al., 2020]. Overproduction of TNF-α and IL-1β leads to tissue damage, multiple organ failure, and finally causes lethal sepsis and the treatment with IS111 indeed reduced sepsis-induced organ damage as studied both by release of ALT and histopathology. Also, these mice were clinically healthier and most importantly, IS 111-treated septic mice had better survival rates.

Macrophages are versatile cells. Their microbicidal function and their participation in the inflammatory response can have immense bearing on the outcome of septicemia [Wegiel B et al., 2015]. In cases of septicemia, studies have shown that a balance of M1 and M2 macrophages may determine the severity of sepsis and survival of infected animals [Renckens R, et al., 2006]. Previous studies using experimental animal models have shown that one of the underlying mechanisms of protection from sepsis is the increased proportion of M2 macrophages [Isabella F. S. 2022], IS 111 can act as an immunomodulator.

On the other hand, it was observed that chronic treatment of IS 111 does inhibit E. coli-induced inflammation for 5 days of treatment [A Brauner, et al., 2001] [Tjabringaa G S ET AL., 2006 & Silva, O. N., C. et al., 2016]. TNF-α and IL-1β are immediately released during the development of systemic inflammatory responses [Hotchkiss, R. S., 2013; Schulte, W., 2013]; this leaves a short therapeutic window for treatment. This rapid event explains why it was observed that acute administration of IS 111 is more efficient and effective than chronic treatment when suppressing the development of inflammation. The treatment significantly reduced the levels of cytokines in lungs, serum and peritoneum and increased the production of cells in peritoneum, as well as lymphocytes at the infection site. IS 111 was able to reduce tissue damage by decreasing the deleterious effects for the organism and contributed to the control of the sepsis and survival of animals; therefore, it is a promising candidate for the development of new drugs. Therefore, agents attenuating pro inflammatory cytokines expression may have potential as treatments for prevention of lethal sepsis [Tobias Schuerholz et al., 2013].

Peritonitis is a common cause of sepsis in humans. Intraperitoneal administration of live E. coli results in a paradigm that resembles a clinical condition commonly associated with septic peritonitis, with diaphragmatic lymphatic clearance, and systemic bacteremia and endotoxemia. This model was used here to investigate the function of Peptide IS 111 in host defense against septic peritonitis. Our results identify for the first time a protective role for Peptide IS 111 in the immune response to abdominal sepsis.

In summary, the administration of IS 111 of 1.2 mg/kg dose reduces the lethality rate and circulating levels of TNF-α, IL-1β and IL-6 in BALB/C mice with enterotoxemia induced by gram negative bacteria 8739™ (E. coli: 5×10⁸ CFU/per mouse) These findings provide clues that IS 111 may be a promising agent for the prevention of systemic inflammatory diseases such as sepsis.

### Example 8: Results of In Vivo Efficacy Activity of Test Peptide IS 111 Using Cecal Ligation and Puncture Induced Peritonitis Animal Model

### Administration of Peptide IS 111 Improves Survival in a Mouse Model of Polymicrobial Sepsis Induced by CLP.

In the studies so far, a system where i.p. injection of high doses of E. coli ATCC 8739 was used to induce sepsis has been used. Administration of high doses of E. coli ATCC 8739 led to a rapid rise in TNF-α and resulted in 100% mortality by 40 h. This model allowed us to study the effects of Peptide IS 111 and dissect its mechanism of action in sepsis. However, the study the effect of Peptide IS 111 in polymicrobial sepsis induced by CLP, which is a more physiological model of sepsis was the next wish to be studied. In this model, the effects of Peptide IS 111 were studied on pro inflammatory cytokines IL-1b, IL-6, IL-12 p70, and TNF-α levels (FIG. 20 a, b, c and d) and anti-inflammatory cytokine IL-10 (FIG. 21) and ultimately survival. Polymicrobial sepsis induced by CLP increased levels of pro inflammatory cytokines, which were significantly reduced in mice that received Peptide IS 111 therapies (FIG. 6A).

In the present study, the effect of Peptide IS 111 on survival of mice subjected to CLP-induced polymicrobial sepsis was also determined. Mice that received 0.9% NaCl solution post-CLP had a median survival time of 22 h, whereas those that received Peptide IS 111 had significantly improved survival after 18 h (FIG. 20) and after 7 days (FIG. 19). In the Peptide IS 111 treatment group, 80% of mice were still surviving when the last death in the disease control group was registered at ~60 h post-CLP (FIG. 20). Mice were monitored until 7 days and the survival percentage in the Peptide IS 111-treated group at this time was 80% (FIG. 19). These data indicate that Peptide IS 111 by virtue of its ability to reduce TNF-α level and prevent organ damage can provide protection in polymicrobial sepsis. Importantly, results from these experiments validate previous observations made using the model of E. coli ATCC 8739-induced septic shock.

The aim of the present study was to investigate anti-inflammatory and immunomodulatory properties of IS 111 doses (0.6 & 1.2 mg/kg) on the control of the systemic inflammatory response, the activation of phagocytes and the control of bacterial growth in a sepsis experimental model.

10-12 male C57BL/6 & BALB/c mice were submitted to the sepsis model by cecal ligation and perforation (CLP group) or laparotomy only (sham group). After 2 h post CLP surgery, the animals received IS 111 (0.6 & 1.2 mg/kg), Intravenously (IV) in & BALB/c mice. The animals received saline in sham control. Blood was isolated for cytokine analysis and other biochemical markers at 18 h and 10 days after CLP. A subset of animals was followed for 10 days for survival assessment, and then behavioral tests were performed. The administration of IS 111 restored the elevation of IL-1β, TNF-α, IL-6, and IL-10 cytokine levels in the sera even after 10 days of post CLP.

After 18 h after the cecal ligation and puncture (CLP), the lungs, spleen and blood were collected to measure the serum cytokines and the animals were killed for the evaluation of cytokines estimation, colony-forming units (CFUs). The results showed that only the test peptide IS 111 treatment inhibited bacterial growth in the peritoneum and inflammatory cellular influx, especially influx of macrophages and neutrophils. However, test peptide IS 111 treatments decreased the pro-inflammatory cytokines in the serum, indicating a systemic anti-inflammatory effect of both.

In the present study, our goals were to investigate the direct influence of proinflammatory cytokines (IL-1β, IL-6, and TNF-α).

### Activity and Vital Parameters:

The activity of mice in the sham, IS 111 treated groups was significantly higher (each P<0.05 and demoted as ***, Table 34) compared to the sepsis-control group 18 hours after CLP. The general activity and body temperature results at 18 h after CLP surgery are shown in Table 34. Both the CLP animals exhibited tachypnea and hypothermia. The test peptide IS 111 treated animals' body temperatures are in range of normal values. [Asma Ahmed, et al., 2018]

TABLE 34
Activity Index of IS 111 treatment and all
groups after 18 h of CLP induced sepsis.

| | Activity index |
|---|---|
| Group | (Grade points) |
| Control | 1.1 ± 0.3 |
| Sham control | 1.8 ± 0.3 |
| Disease control (CLP) | 4.4 ± 0.8 |
| CLP + IS111-0.6 mg/Kg | 2.8 ± 0.5*** |
| CLP + IS111-1.2 mg/Kg | 2.2 ± 0.7*** |

Experiment 1 Survival study:
IS 111 Treatment Enhanced Survival of Mice after Induction of Sepsis.

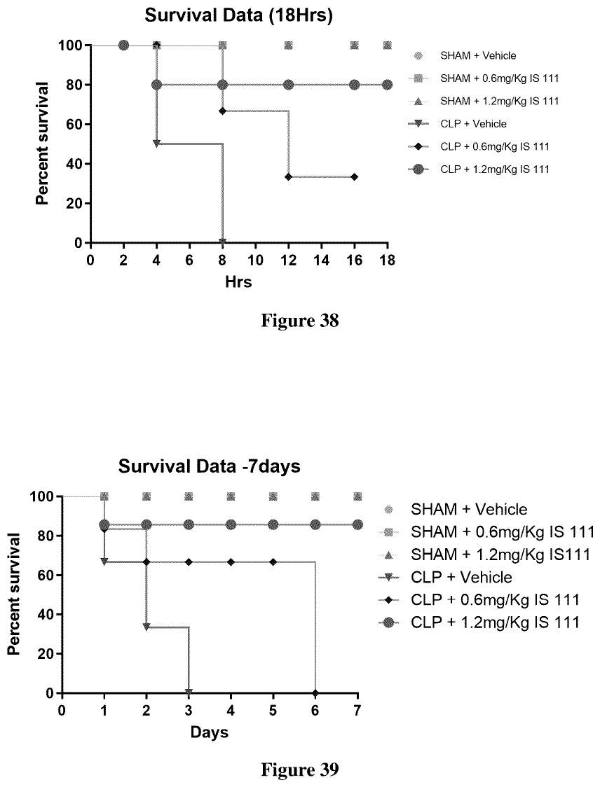

The Kaplan-Meier curve for survival analysis of mice subjected to polymicrobial sepsis and treated with test peptide IS 111 (0.6 & 1.2 mg/kg) showed lower mortality after sepsis when compared with non-treated septic mice (FIGS. 38 and 39). Peritonitis induced by CLP with a 21-gauge needle results in 100% lethality at day 8 [Yona Kalechman, Uzi Gafter, et al., 2002] CLP was performed as described in the methods section and animals were Intravenously (IV) injected with various doses of IS 111 or vehicle at 2 h after CLP procedure and then mice were observed for 18 h (FIG. 38) and subset of animals are continued for observation for 7 days (FIG. 39). No significant difference was observed when IS 111 was injected at the time of injury (time zero). Notably, treatment with IS 111 18 h decreased survival, compared with NS-treated mice [Konstantin Tsoyi, et al., 2009; Kim, Y. K., 2017].

FIG. 38 represents the short synthetic peptide IS 111 increases survival after 18 h of the treatment-after Sepsis Induction. FIG. 39 represents the short synthetic peptide IS 111 increases survival after 7 days of the treatment-after Sepsis Induction. Kaplan-Meier curve for survival analysis of mice subjected to polymicrobial sepsis and treated with peptide IS 111. Data are shown as mean±SD of six mice in each group, analyzed by one-way ANOVA and Tukey post hoc tests. *p<0.05 in relation to CLP+saline group.

### Experiment 2: Acute Hyper Inflammation Study: Cytokine Estimation in Serum:

To investigate the underlying potential mechanism of the protective effect of IS 111, the levels of the representative proinflammatory cytokines TNF-α, IL-1β and IL-6 in the peritoneal cavity, lungs and spleen of severe CLP mice were measured. In blood sera the levels of the representative cytokines TNF-α, IL-1β, IL-6, IL12 and IL-10 were measured by ELISA. Both intravenous and subcutaneous administration of IS 111 markedly decreased the TNF-α, IL-1β and IL-6 levels in peritoneal fluid and serum 18 h after CLP surgery compared with CLP mice. More importantly, the concentrations of both TNF-α, IL-1β and IL-6 were lower after the treatment IS 111 than after intravenous administration, whereas IL-10 levels increased slightly only at high dose of IS 111 (1.2 mg/kg). Pro-inflammatory macrophages with an M1 phenotype play an important role in mediating inflammation. The same trend was observed in samples collected after 10 days with treatment of IS 111 daily for 5 days. [Burgelman, M et. al, 2021]

Effect of IS 111 (0.6 & 1.2 mg/Kg-IV) Administrations on Proinflammatory Cytokines Levels After CLP Surgery:

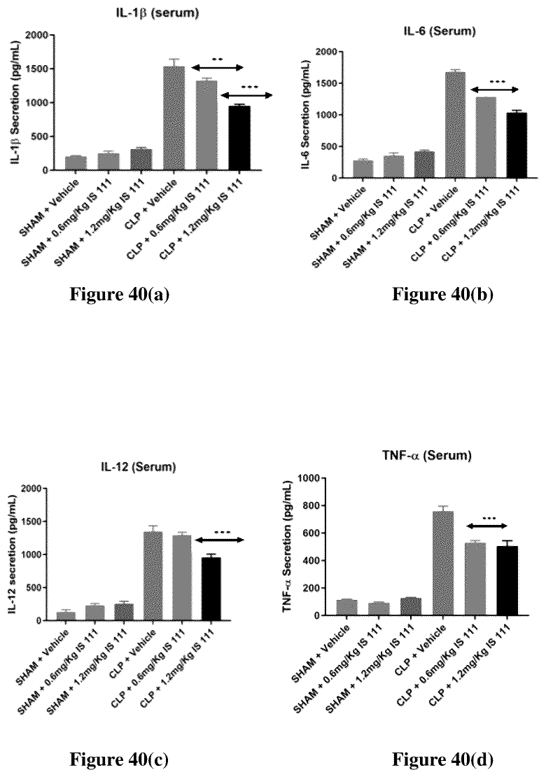

Organ injury observed in sepsis is due to the explosive release of cytokines into the serum/plasma. It was therefore sought to determine the serum levels of cytokines following CLP and their response to IS 111 treatments. The classic cytokines produced in the initial period of an inflammatory insult are TNF-α, IL-1β IL-6 and IL-12, which are followed by overproduction of IL-10. TNF-α and IL-1β levels were undetectable in the serum at 1 h post-CLP. Maximal levels of these cytokines were found at 6 and 12 h. The inflammatory response was not persistent, and it gradually declined, until at 24-48 h after CLP it was only minimal and so, in the present study, the samples were collected at 4 h and 18 h. [Wendy E. Walker, 2021] The results are as follows. TNF-α, IL-1β IL-6 and IL-12 were increased in the sepsis-control (CLP) group and significantly decreased in the IS 111 −1.2 mg/kg compared to the sepsis-control group (P<0.001) and the trend of TNF-α levels at 18 h in treatment groups (FIG. 40) intravenously. FIG. 40 shows the detection of cytokines (IL-1β, IL-6, IL-12 & TNF-α) in the serum sample of animals after CLP surgery, induction of sepsis and short synthetic peptide IS 111 treatment, after 18 hrs of polymicrobial sepsis.

Levels of proinflammatory cytokines IL-1β, IL-6, IL-12, TNF-α in the serum (a, b, c & d) in serum samples at 18 h after induction of polymicrobial sepsis and treatment with synthetic peptide IS 111 at dose of (0.6 & 1.2 mg/kg). Data are shown as mean±SD of six mice in each group, analyzed by one-way ANOVA and Tukey post hoc tests. *p<0.05 in relation to CLP+saline group.

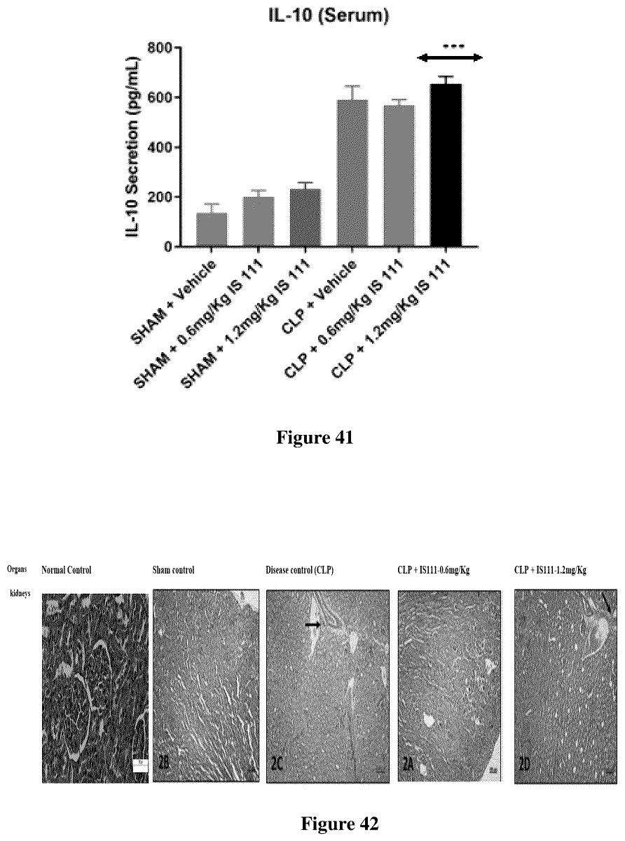

FIG. 41 shows detection of cytokines (IL-10) in the serum sample of animals after CLP surgery, induction of sepsis and short synthetic peptide IS 111 treatment, after 18 hrs of polymicrobial sepsis.

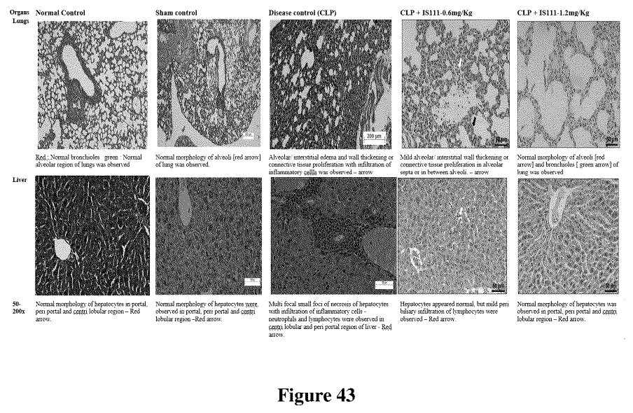

Levels of anti-inflammatory cytokine IL-10 in serum samples at 18 h after induction of polymicrobial sepsis and treatment with synthetic peptide IS 111 at dose of (0.6 & 1.2 mg/kg) (FIG. 43). Data are shown as mean±SD of six mice in each group, analyzed by one-way ANOVA and Tukey post hoc tests. *p<0.05 in relation to CLP+saline group.

### Histopathological Changes of Lung Kidney & Liver Tissues:

### IS 111 Treatment Ameliorated Organ Injury in CLP Mice:

Organ damage is a leading cause of death in patients with sepsis. Thus, whether the organ protection afforded by IS 111 in CLP mice was investigated. No significant changes were observed in the body weight of the animals and in relation to the organs weight, besides, no macro- or microscopic alteration was detected in the brain, heart, lungs, liver, kidney, and spleen. All the tissues from different experimental groups were harvested after 18 h, considering the early phase of immunosuppression and most of the animals of the CLP group are not survived while the other treated groups (IS 111 −0.6 mg/kg and IS 111 −1.2 mg/kg groups) lived longer, to assess lung and liver damage, histological examinations. The presence of congestion in the groups analyzed was due to the euthanasia procedure. Necrosis was not observed in any analyzed organs.

Briefly, lung and liver tissues were fixed in buffered 10% formaldehyde and then embedded in paraffin. The embedded tissue samples were sectioned (5 μm) and stained with haematoxylin and eosin to examine general histological features. These investigations showed that CLP-induced sepsis in mice caused hepatic inflammatory cellular infiltration, hepatic steatosis, and hepatic fibroplasia in the portal tract. A semi-quantitative scoring system was used. For liver tissue evaluation, hepatocyte degeneration and portal/lobular inflammation were scored (each 0-3), Lung injury scores were determined by assessing neutrophil infiltration, hemorrhage, necrosis, congestion and edema as previously described. The score of each tissue sample represented the mean score of ten different fields. The stained tissue sections were evaluated under a light microscope (Eclipse E200-LED; Nikon, Kawasaki, Japan) at ×200 magnification.

The general architectures of the lung, kidney and liver in the sham and normal control groups were of normal histological structure (FIG. 42-43). There was also no statistically significant difference between both the sham and Normal control groups (P<0.01). However, lung tissue in the CLP-control showed histopathological changes in the alveolar walls (FIG. 43). Also, interstitial edema, infiltration of polymorphnuclear leukocytes and monocytes, hemorrhage, vascular congestion, and cellular hyperplasia were observed, and the tissue damage was more prominent in the disease control group. Inflammatory cell types were generally neutrophils and macrophages. In the lungs, congestion and neutrophil infiltration were observed in both groups. Neutrophil infiltration into the alveolar space was not observed in any case, and no traces of pneumonia were noted in any of the lung samples. The morphologic study showed that the lungs of CLP mice were damaged. Severe oedema, wider interalveolar septa, severe alveolar haemorrhage, and extensive inflammatory cell infiltration was observed. But the lungs and livers of the IS 111 −0.6 mg/kg group and IS 111 −1.2 mg/kg group had normal histological structure, when compared with the CLP group (FIG. 43). Mild lung oedema, haemorrhage, and inflammatory cell infiltration were seen in the IS 111 −0.6 mg/kg treatment group. Furthermore, it was evident that IS 111 (1.2 mg/kg) reduced these injuries by reducing edema and macrophage infiltration. Histological evaluation of lung tissue revealed that IS 111 reduced macrophage infiltration and alleviated lung tissue damage (FIG. 43). In addition, compared with the blank control group, the renal tissue of the model group did not show obvious substantial lesions, only the changes of renal function were observed. Compared with the model group, a small part of the renal tubules had proteinuria in the kidney tissue section and rare tubeuria occurred after IS 111 administrations (FIG. 42). Normal morphology of glomerulus [red arrow] and tubules [green arrow] was observed in cortex region in control animals.

FIG. 42 shows photographs of representative sections of kidney sections were prepared and stained with H&E. visualized at 200× magnification are shown. Data shown in mean±SEM from 3-4 mice of all groups of CLP induced sepsis, treatments, and control animals-IV route-18 h.

FIG. 43 shows photographs of representative sections of liver (a) and lungs (b) sections were prepared and stained with H&E. visualized at 200× magnification are shown. Data shown in mean±SEM from 3-4 mice of all groups of CLP induced sepsis, treatments, and control animals—IV route—18 h.

CLP model induced polymicrobial infection (blood cultures positive for Escherichia coli, Streptococcus bovis, Proteus mirabilis, Enterococcus, and Bacteroides fragilis) and bacteremia (peritoneal cavity fluid positive for the above microbes as well as Streptococcus viridians and Clostridium sporogenes) and a 70% mortality rate [Konstantin Tsoyi, et al., 2009]. Mildly ill mice sacrificed 10 hours following CLP demonstrated the early hyperdynamic phase of sepsis (increased blood flow to organs, hyperinsulinemia, and hyperglycemia) [Burgelman, M et al., 2021; Hotchkiss, R. S., et al., 2013]. Thus, in the present study sera was collected after 18 h of CLP surgery to know the effect of IS 111 treatments on late hypodynamic phase of sepsis.

The pro-inflammatory response is also characterized by significant increases in cytokines TNFα and IL-6 and remain elevated over an 8-hour period [Schulte, W., J. Bernhagen et al., 2013; Matsukawa, A., 2003; KYUNG-JUN JANG et al., 2016 & Gil, M., 2016]. Several studies have demonstrated the importance of an early pro-inflammatory response in the progression of sepsis [Schuerholz et al. 2013; Singleton K, et al., 2003; Silva, O. N., 2016]. To screen the host response to CLP, as part of this the following estimations are done in sera and the results are mentioned as in the results section. [Stefan Wirtz et al., 2006; Yung-Yang Liul 2008]. As only a fraction of patients with severe sepsis and septic shock displays signs of DIC, this level of effectiveness is not surprising [Vincent, J. L. et al., 2005; Levi, M. 2010 & 1999]. In the present study a strategy of controlling bacterial multiplication along with inhibition of excessive proinflammatory cytokines by use of peptide IS 111 treatments might be more effective in controlling human sepsis.

Recent studies demonstrated that systemic levels of both the proinflammatory cytokines and IL-10 are correlated directly with severity of illness [Yona Kalechman, Uzi Gafter, et al., 2002]. However, during septic peritonitis induced by cecal ligation and puncture (CLP), 3 neutralizing IL-10 was associated with an increased mortality [Grace Y. 1999] In this study, it was shown that in mice subjected to cecal ligation and puncture (CLP), treatment with IS 111 2 h after CLP significantly increased survival of septic mice. This was associated with a significant decrease in serum IL-10 and in IL-10 secretion by peritoneal macrophages 24-48 h after CLP. At that time, the ability of these cells to secrete TNF-α, IL-6, IL-12 and IL-1β was restored in IS 111-treated mice for 5 days and this reflects in survival rate which observed for 10 days and also in tissue damage. Recent data of Song et al. suggest that at later time points after the onset of infection, IL-10 may have a net detrimental effect on host antimicrobial clearance mechanisms. In a cecal ligation model, when administered 2 h after CLP, this treatment was protective. This data indicates the critical importance of timing of manipulations that affect IL-10 activity in sepsis.

Moreover, IS 111 treatment ameliorated bacterial clearance in the peritoneum and blood and decreased severe multiple organ damage, as indicated by clinical chemistry. Furthermore, myeloperoxidase levels in the lung of IS 111-treated mice, an indirect means of determining the recruitment of neutrophils, were significantly decreased. It is suggested that non immunogenic peptide IS 111, with the capacity to inhibit IL-10 and stimulate macrophage functions, may have clinical potential in the treatment of sepsis, provided they are administered during the phase of sepsis characterized by immune suppression.

### Summary—In Vivo Studies—CLP Sepsis Induction Model:

From above in vivo study results, it was concluded that dose dependent response of the peptide was observed, and Peptide IS 111 demonstrates promoting properties of immune modulation by upregulating pro-inflammatory cytokines, IL-1β, and TNF-α expression and anti-inflammatory cytokines IL-10 levels. Considering the beneficial activity demonstrated by Peptide IS 111, in multiple steps in CLP induced sepsis in-vivo animal model, utilization of this drug for treatment of COVID-19 or sepsis is proposed.

### Discussion of the Present Study:

Sepsis is a condition described by systemic hyperinflammation induced because of excessive production of proinflammatory cytokines such as TNF-a, IL-1b, and IL-6. Its causes are uncontrolled bacteremia resulting from situations of pneumonia, peritonitis, and surgical procedures. Specifically, in the majority of cases, infection is caused by Gram-negative bacteria and LPS from the outer membrane of the bacteria overstimulates the host immune response. Treatment for sepsis consists of eradication of infection through early and aggressive treatment with appropriate antibacterials. However, despite advances in the development of powerful antibiotics, sepsis is still life-threatening. Therefore, there remains a need for development of novel antisepsis therapies combining antibiotic therapy with the application of a nonsteroidal anti-inflammatory regimen. In the present invention, the Peptide IS 111 is designed antimicrobial peptide (dAMP) with immunomodulatory activity designated as Innate Defense Regulator (IDR) peptides. In general, the anti-microbial activity of AMP's might be as follows, where the AMPs must interact with membranes as part of their direct antibacterial mechanism (or mechanisms) of action, leading to membrane perturbation, disruption of membrane associated physiological events such as cell wall biosynthesis or cell division, and/or translocation across the membrane to interact with cytoplasmic targets and destroy the cell by changing membrane conductance and altering intracellular function and Alterations in membrane structure results in the reorientation of peptide molecules in the membrane culminating in eventual pore formation and lysis of the target microbe.

Synthetic peptide analogs which are designed a short peptide with dual action of anti-microbial activity with Immunomodulatory action, which can also act as agents of eukaryotic cell proliferation. Amphipathic peptides that promote lysis of transformed cells will, at lower concentrations, promote cell proliferation in some cell types. This stimulatory activity is thought to depend on the channel forming capability of the amphipathic peptides, which somehow stimulates nutrient uptake, calcium influx or metabolite, release, thereby stimulating cell proliferation (Jaynes, J. M. Drug News & Perspectives 3:69 [1990]; and Reed, W. A. et al. Molecular Reproduction and Development 31:106 [1992]). Thus, at a given concentration, these peptides stimulate or create channels that can be beneficial to the normal mammalian cell in a benign environment where it is not important to exclude toxic compounds.

The Peptide IS 111 at a concentration equal to 5×MIC was rapidly bactericidal, achieving complete elimination of both test bacterial strains within 3 h. All of the time-kill data obtained with the test compound Peptide IS 111 showed its antibacterial activity to be time- and concentration-dependent. Peptide IS 111 displays rapid bactericidal activity and a low tendency for the development of resistance. Taking all the study results into account, it is believed that Peptide IS 111 has the potential to serve a as backbone molecule for the development of new anti-infective therapies. In follow-up studies, it will be investigated whether these in vitro time-kill data are predictive of in vivo efficacy.

In this study, septicemia was induced in mice by injection of very high doses (5×10⁸ CFU) of Gram-negative E. coli ATCC 8739 i.p IP (Intraperitoneal). Such high doses of bacteria were potent inducers of proinflammatory cytokines (TNF-a, IL-1b, and IL-6) and organ damage, which are hallmarks of septicemia. Also, apart from inducing a cytokine storm, high doses of E. coli ATCC 8739 resulted in 100% fatality by 40 h. Therefore, this is a robust model to study excessive inflammation and septicemia as has been reported previously. In this system the effect of Peptide IS 111 which comprises several immunomodulatory proteins was tested. It was found that Peptide IS 111 significantly reduces levels of TNF-α, IL-1b, and IL-6. Both IL-1b and IL-6 have been shown to be elevated during septicemia. However, the role of IL-6 in experimental sepsis models is controversial as IL-6 has both anti- and proinflammatory properties. Blockade of IL-6 has been shown to be beneficial in sepsis as well as other inflammatory diseases) indicating a positive correlation between elevated IL-6 levels and sepsis severity. Thus, Peptide IS 111 may reduce the inflammation and thereby decrease toxicity of sepsis by inhibiting TNF-α, IL-1b, and IL-6 production. Also, these mice were clinically healthier and most importantly, Peptide IS 111-treated septic mice had better survival rates.

In a separate mouse model of polymicrobial sepsis induced by CLP, Peptide IS 111 reduced TNF-α, attenuated liver and kidney damage, prevented sepsis-induced depletion of monocytes and lymphocytes, and ultimately increased survival. The protective effects of Peptide IS 111 were, therefore, demonstrated in two models of sepsis. Ability to reduce TNF-αand attenuate organ damage in CLP-induced sepsis correlates with increased survival as has been demonstrated previously. Peptide IS 111 was able to consistently reduce elevated levels of TNF-α in E. coli ATCC 8739-induced and CLP-induced sepsis when administered therapeutically.

The former is a model where LPS is likely to play a major role (73, 74) and the latter is a model of polymicrobial sepsis. Peptide IS 111 works efficiently to reduce mortality in both model systems. Timely administration of antibiotics controls bacterial replication, but cannot undo the damaging effects of the systemic cytokine storm. A strategy of controlling bacterial multiplication along with inhibition of excessive proinflammatory cytokines and DIC by use of agents such as Peptide IS 111 might be more effective in controlling human sepsis. Macrophages are versatile cells, their microbicidal function and their participation in the inflammatory response can have immense bearing on the outcome of septicemia. Use of Peptide IS 111 in a model of sepsis not only helped us to identify a potential candidate for sepsis therapy but also provided novel insights into the mechanism of action of a mycobacterial virulence factor. Results from our present invention show that Peptide IS 111 is a potent inhibitor of proinflammatory cytokines when given therapeutically in mouse models of E. coli ATCC 8739-induced and CLP-induced septicemia. Also, Peptide IS 111 was found to be a potent inducer of IL-10, markers for M2 macrophages.

A therapy which inhibits the production of inflammatory cytokines perhaps has a better chance of success along with antibiotics. Thus, its ability to dampen harmful responses and elevate protective responses in sepsis makes Peptide IS 111 a promising candidate for antisepsis therapy.

Although the present invention has been described in detail with specific features, those skilled in the art might well identify that this description is only for a preferred embodiment and does not limit the scope of the present invention. It will be obvious to the skilled person to make various amendments to the invention described here in the document. To the extent that these various changes, modifications and alterations do not depart from the scope of the present invention, they are intended to be encompassed therein.

In view of the examples provided herein (IL-1β, TNF-α, IL-12, IL-6, inhibition), the Peptide IS 111 can be used in following areas: for treating, preventing or reducing the severity of one or more symptoms of many diseases many diseases where infections and immune functions play a central role, such as Temporary acquired immune deficiencies, systemic lupus erythematosus (SLE), Rheumatoid arthritis (RA), Multiple sclerosis (MS), Severe combined immunodeficiency (SCID), chronic respiratory diseases, and among others particularly sepsis and COVID-19, in a subject in need thereof, comprising administering to the subject a therapeutically effective amount of said formulation, wherein said formulation is capable of altering level of cytokines and anti-microbial activity.

The present invention describes the short synthetic Peptide IS 111 demonstrates promoting properties in sepsis and COVID-19 by upregulating pro-inflammatory cytokines, IL-1β, and TNF-α expression.

Taken together, clinical and experimental evidence suggests that septic patients are not necessarily in an overactivated immunological state. Therefore, any immunomodulatory therapy should be based on measurable immune functions to determine which patients may benefit from such therapies. It is believed that the use of the nontoxic short synthetic Peptide IS 111, has potential in the clinical treatment of sepsis if given during the immune suppressive state of septic & covid-19 patients and in many diseases where immune functions play a central role.

### Highlights of the Study:

Effects of IS 111 on as anti-microbial agent and immune modulatory activity have been reported for first time.
It is concluded that IS 111 exerts anti-bacterial and anti-inflammatory effects in vitro and in vivo.
It is confirmed the anti-inflammatory activity of IS 111 in macrophage cell line and peritoneal macrophages.
Acute toxicity profile of IS 111 peptide was established.
IS 111 suppress or restores the pro inflammatory cytokines and increases anti-inflammatory cytokines in early immune suppression of lethal sepsis models: Cecal ligation and puncture and E. coli induced peritonitis sepsis models.
IS 111 reduces the organ damage and restores the morphology of the lungs, kidneys and liver of sepsis induced the organ damage in mice.

### Study Impression:

The current treatment for sepsis is the administration of antibiotics and supportive care measures for the patient. The present invention showed here that Peptide IS 111 treatments improved the local innate immunity, decreased bacterial growth and, concomitantly, avoided the systemic exacerbated inflammatory response usually observed in sepsis, and leading to a better prognosis.

### Summary of all the Studies & Conclusion

TABLE 35
The protective effects and mechanisms of IS 111peptide
in sepsis -related animal or cell model:

| In vivo/ | | Therapeutic | |
|---|---|---|---|
| vitro | Animal or cell model | dose | Effects and Mechanisms. |
| In vivo | CLP-induced sepsis in | 0.6 mg/kg/1.2 | ↑Survival rate; ↓TNF-α, IL-1β, IL- |
| | male BALB/c mice | mg/kg via., | 6 and IL-12, &↑IL-10 in serum; |
| | septic mouse model | Intravenous | ↓Lung damage and injury scores; |
| | (8-10 weeks) | route −18 h. | ↓injury of pulmonary |
| | | | permeability: ↓ TNCC, |
| | | | macrophages & neutrophils; |
| | | | ↓bacterial counts; ↓liver & kidney |
| | | | tissue injury and injury score. |
| In vivo | E. coli-induced | 0.6 mg/kg/1.2 | ↑Survival rate; ↓TNF-α, IL-1β, IL- |
| | peritonitis sepsis in | mg/kg via., | 6 and IL-12, &↑IL-10 in serum; |
| | male BALB/c mice | Intravenous | ↓Lung damage and injury scores; |
| | septic mouse model | route −18 h. | ↓injury of pulmonary |
| | (10-12 weeks) | | permeability: ↓ IL-6 and TNF-α in |
| | | | peritoneal fluid (PLF); ↓ TNCC, |
| | | | macrophages & neutrophils; |
| | | | ↓bacterial counts; ↓liver &kidney |
| | | | tissue injury and injury score. |
| In vivo | Acute toxicity | Single dose of 0.6/ | No mortality; no changes on body |
| | | 2.4/4.8 mg/kg | weight and organ weights in |
| | | via., Intravenous | treated groups. |
| | | route. | |
| In vitro | LPS -stimulated | 3.125/6.25/12.5 | ↓Cytotoxicity; ↓ NO production; ↓ |
| | RAW 246.7 cell lines | μg/mL. | IL-12, IL-β, IL-6, and TNF-α and |
| | of murine | | ↑ IL-10 levels;); ↓ mRNA |
| | macrophages | | expressions of IL-12, IL-β, IL-6, |
| | | | and TNF-α. block activation of |
| | | | MAPKs. |
| In vitro | LPS -stimulated | 1.56/3.12/ | ↓Cytotoxicity; ↓IL-6, IL-1β, and |
| | BALB/C mice | 6.25/12.5/25/ | TNF-α and ↑ IL-10 levels. |
| | peritoneal | 50/100 μg/mL. | |
| | macrophages | | |
| In vitro | Anti-microbial | 1.56/0.78/0.39/ | MIC; IS 111 has showed good |
| | studies: Minimum | 1.56 μg/mL. | antimicrobial activity at the |
| | inhibitory | | concentration of 1x MIC |
| | concentration (MIC) | | (1.56 μg/ml) against S. aureus, 5x |
| | & Time-killing assay | | MIC (0.78 μg/ml) against P. |
| | of peptide IS 111 | | aeruginosa, at 2 h, 1x MIC |
| | against S. aureus, P. | | (0.39 μg/ml) against E. coli at 3 h |
| | aeruginosa, E. coli & | | and 1x MIC (1.56 μg/ml) against |
| | K. Pneumoniae. | | K. pneumoniae and at 12 h showed |
| | | | the completed reduction of initial |
| | | | inoculum at 24 h incubation. |

### Dose Calculations of Test Peptide (IS 111) and Standard Drug Used in the In Vivo Animal Studies.

### Preparation and Administration of Test Peptide Drug Doses—In Vivo Studies:

The test compound is provided in a 10 mg quantity in a vial and prepare the stock solution.
Prepare the stock solution containing 2 mg/l ml (10 mg is diluted in 5 ml of saline) and name it as STOCK SOLUTION-I.
From the above stock solution, three working solutions to the required dose for mice as per group according to the body weight were calculated and prepared.
The acute toxicity and in vivo test dose preparation are as follows.

TABLE 36
The preparation IS 111 - test doses from the stock solution.

| Test dose | Preparation: |
|---|---|
| 0.6 mg/kg | 0.06 mg/mL was prepared form the stock solution |
| | (8.61 mg/mL) by diluted 0.035 μL of stock in 4.965 mL of |
| | 0.9% NaCl. |
| 1.2 mg/kg | 0.24 mg/mL was prepared form the stock solution |
| | (8.61 mg/mL) by diluted 139 μL of stock in 4.861 mL of |
| | 0.9% NaCl. |
| 4.8 mg/kg | 0.48 mg/mL was prepared form the stock solution |
| | (8.61 mg/mL) by diluted 279 μL of stock in 4.721 mL of |
| | 0.9% NaCl. |

### Preparation of Standard Drug Doses—In Vivo Studies:

The standard drugs dose is prepared by using the following formula: [Jang-Woo Shin et al., 2010 & Anroop B. Nair, et al., 2016]

Animal dose (mg/kg)=HED (mg/kg)×conversion factor.
HED: Human equivalent dose; Conversion factor from human to mouse is 12.33.

All the animals will receive the same volume of the test dose 0.1 mL by subcutaneously and intravenously. Repeat the procedure for preparation of test drug daily for fourteen days in acute study and in vivo efficacy studies the test drug was prepared freshly at the time of administration based on the requirement.

### Conclusion of the Study:

Sepsis, a life-threatening disease with a high mortality rate due to infection accompanied by systemic inflammation (Centers for Disease Control and Prevention, USA). The rapid increase in the incidence of multidrug-resistant infections today has led to enormous interest in antimicrobial peptides (AMPs) as new therapeutic strategies for developing unusual antibiotics with immune modulating activity. Thus, the development of immunomodulatory therapeutic approaches is therefore one of the principal areas emphasized in developing modern forms of treatment for sepsis. Host defense anti-microbial peptides are considered potent drug molecules as reported for their anti-microbial properties along with immunomodulatory activity.

In this study, Peptide IS 111, a HDP, which is a 7 amino acids and derivative of designed anti-microbial peptide (dAMPs), was selected as a purposeful molecule that could be used in controlling infection and further synthesized. The test peptide IS 111 exhibits potent antimicrobial and immunomodulatory properties both in vitro and in vivo. In vitro, the peptide effectively killed a panel of representative bacterial strains, includes S. aureus, E. coli and P. aeruginosa as well as Raw 246.7 mouse macrophages cell lines. Despite displaying clear in vitro antimicrobial activity toward gram-positive and -negative bacteria, IS 111 showed no cytotoxic activities against primary macrophages cells, and in acute toxicity tests, no adverse reaction was observed at any of the concentrations.

Moreover, this peptide was challenged here in an in vivo sepsis model, and the immune response was also analyzed. This peptide also reduced the mortality of mice infected with Gram-negative strain E. coli and CLP induced peritonitis/sepsis by 80% compared with that of diseased control animals (treated with normal saline [NS]); these data suggest that IS 111 prevents the start of sepsis and thereby reduces mortality. In addition, IS 111 peptide was capable of modulating innate immunity by stimulating leukocyte recruitment to the site of infection and repressing the levels of pro-inflammatory cytokines IL-12, IL-1β, IL-6 and TNF-α, while suppressing an excessive and potentially harmful inflammatory response by increasing synthesis of anti-inflammatory cytokines such as IL-10, both in peritoneal macrophages and serum. In addition, IS 111 restores liver and kidney enzymes and reduces organ injury. Moreover, short-term treatment (Single dose) of IS 111 peptide results in a suppression of pro-inflammatory cytokines expression, which can effectively protect mice from sepsis-related systemic inflammation and mortality. These data suggest that IS 111 is an HDP-dAMP, that directly kills bacteria and further helps resolve infections through its immune modulatory properties, includes its ability to dampen harmful immune responses and elevate protective responses in sepsis. Thus, IS 111 Peptide represents a new approach of anti-infective therapeutics and is a promising candidate for antisepsis therapy.

The foregoing is only a description of the preferred examples of the present disclosure and the applied technical principles. It should be appreciated by those skilled in the art that the protective scope of the present disclosure is not limited to the technical solutions formed by the particular combinations of the above technical features. The protective scope should also cover other technical solutions formed by any combinations of the above technical features or equivalent features thereof without departing from the concept of the present disclosure, such as, technical solutions formed by replacing the features as disclosed in the present disclosure with (but not limited to), technical features with similar functions.

It will be obvious to those skilled in the art to make various changes, modifications and alterations to the invention described herein. To the extent that these various changes, modifications and alteration do not depart from scope of the present invention, they are intended to be encompassed therein.

### REFERENCES

A Brauner, et al., Escherichia coli-induced expression of IL-1α, IL-1β, IL-6 and IL-8 in normal human renal tubular epithelial cells. Clin Exp Immunol. 2001; 124 (3): 423.
Abreu A. G., et al., The serine protease Pic from enteroaggregative Escherichia coli mediates immune evasion by the direct cleavage of complement proteins. J. Infect. Dis. 2015; 212:106.
Ajaz Ahmad, et al., Therapeutic potential of Rhododendron arboretum polysaccharides in an Animal Model of Lipopolysaccharide-Inflicted Oxidative Stress and Systemic Inflammation. Molecules 2020, 25, 6045.
Ali, T., Kaitha, S. et al., Clinical use of anti-TNF therapy and increased risk of infections. Drug Health. Pat. Saf. 2013; 5, 79.
A-ling Tang, et al., Prognostic role of elevated VEGF in sepsis: A systematic review and meta-analysis. Front Physiol. 2022; 13:941257.
Amisha Barochia. et al., Eritoran tetrasodium (E5564) treatment for sepsis: review of preclinical and clinical studies. Expert Opin. Drug Metab. Toxicol.2011; 7 (4): 479.
Amit Kumar et al., Evaluation of the immunomodulatory and anti-inflammatory activity of Bakuchiol using RAW 264.7 macrophage cell lines and in animal models stimulated by lipopolysaccharide (LPS) International Immunopharmacology. 2021; 91: 107264.
Amit Pant et al., Advances in sepsis diagnosis and management: a paradigm shift towards nanotechnology. Journal of Biomedical Science, 2021; 28:6.
An H, et al., Up-regulation of TLR9 gene expression by LPS in mouse macrophages via activation of NF-kappaB, ERK and p38 MAPK signal pathways. Immunol Lett 2002; 81:165.
Anasuya Patel, et al., Immunomodulatory Effect of Doxycycline Ameliorates Systemic and Pulmonary Inflammation in a Murine Polymicrobial Sepsis Model Inflammation, 2020; 43, 3.
Andaluz-Ojeda, et al., A combined score of pro- and anti-inflammatory interleukins improves mortality prediction in severe sepsis, Cytokine, 2012; 57, 3, 332.
Antonelli, M. Sepsis and septic shock: pro-inflammatory or anti-inflammatory state? J. Chemother. 1999; 11, 536-540.
Arash Karimi, et al., Therapeutic effects of curcumin on sepsis and mechanisms of action: A systematic review of preclinical studies. Phytotherapy Research. 2019; 1.
Archer, N. K. et al., Staphylococcus aureus biofilms: properties, regulation, and roles in human disease. Virulence 2, 2011; 445-459.
Asma Ahmed, et al., Mycobacterium tuberculosis PPE18 Protein Reduces Inflammation and Increases Survival in Animal Model of Sepsis. The Journal of Immunology, 2018, 200.
Bakhtiari, K et al., Prospective validation of the international society of thrombosis and haemostasis scoring system for disseminated intravascular coagulation. Crit. Care Med. 2004; 32, 2416-2421.
Banyer, et al., Cytokines in innate and adaptive immunity. Rev. Immunogenet. 2000; 2, 359-373.
Benjamin G. et. al., Cytokine storm and sepsis disease pathogenesis. Semin Immunopathol. 2017; 39:517.
Bharath Kumar, et al., Sepsis Epidemiology and Outcomes in Asia: Advancing the Needle. Am J Respir Crit Care Med 2022; 206 (9): 1059.
Bhattacharyya S, et al., Macrophage glucocorticoid receptors regulate toll-like receptor 4-mediated inflammatory responses by selective inhibition of p38 MAP kinase. Blood 2007; 109:4313.
Bin Li a, et al., Antimalarial artesunate protects sepsis model mice against heat-killed Escherichia coli challenge by decreasing TLR4, TLR9 mRNA expressions and transcription factor NF-κB activation. International Immunopharmacology. 2008; 8, 379.
Bingging yang. et al., Targeted Therapeutic Strategies in the Battle Against Pathogenic Bacteria. Front Pharmacol. 2021; 12:673239.
Blanco J, et al., Incidence, organ dysfunction and mortality in severe sepsis: a Spanish multicentre study. Crit Care 2008; 12: R158.
Bone, R. C. et al., Definitions for sepsis and organ failure and guidelines for the use of innovative therapies in sepsis, the ACCP/SCCM Consensus Conference Committee. American College of Chest Physicians/Society of Critical Care Medicine. Chest, 1992; 101, 1644-1655.
Boomer J S, et al., The changing immune system in sepsis: is individualized immunomodulatory therapy the answer? Virulence. 2014.
Boorn K L, et al., antimicrobial activity of honey from the stingless bee Trigona carbonaria determined by agar diffusion, agar dilution, broth microdilution and time-kill methodology. J Appl Microbiol. 2010 May; 108 (5): 1534-43.
Braff M H, et al., Structure-function relationships among human cathelicidin peptides: dissociation of antimicrobial properties from host immunostimulatory activities. J Immunol 2005; 174:4271.
Burgelman, M., et al., Extracellular Vesicles: A Double-Edged Sword in Sepsis. Pharmaceuticals 2021, 14, 829.
C. A. Dinarello, et al., Infection, fever, and exogenous and endogenous pyrogens: some concepts have changed, Journal of Endotoxin Research, 2004; 10, 4, 201.
Cadena J, et al., Clindamycin-resistant methicillin-resistant Staphylococcus aureus: epidemiology and molecular characteristics and associated clinical factors. Diagn Microbiol Infect Dis. 2012 September; 74 (1): 16.
Cai J, et al., A Novel Anti-Infective Peptide BCCY-1 With Immunomodulatory Activities. Front. Immunol. 2021; 12:713960.
Carly J. Paoli, et. al., Epidemiology and Costs of Sepsis in the United States—An Analysis Based on Timing of Diagnosis and Severity Level. Crit Care Med. 2018 December; 46 (12): 1889.
Carolin Fleischmann et. al., Global incidence and mortality of neonatal sepsis: a systematic review and meta-analysis. Arch Dis Child. 2021; 106:745-752.
Centers for Disease Control and Prevention-Sepsis. Aug. 9, 2022. Available at: https://www.cdc.gov/sepsis/index.html.
Centers for Disease Control and Prevention. Staphylococcus aureus resistant to vancomycin—United States. MMWR Morb Mortal Wkly Rep. 2002; 51:565.
Charalambos A. et. al., Pro-versus Anti-inflammatory Cytokine Profile in Patients with Severe Sepsis: A Marker for Prognosis and Future Therapeutic Options. The Journal of Infectious Diseases 2000; 181:176.
Chen H, et al., Early lactate measurement is associated with better outcomes in septic patients with an elevated serum lactate level. Crit Care. 2019; 23 (1): 351.
Chen H H, et al., Inhibitory effects of artesunate on angiogenesis and on expressions of vascular endothelial growth factor and VEGF receptor KDR/flk-1. Pharmacology 2004; 71:1.
Chen L, et al. Inflammatory responses and inflammation-associated diseases in organs. Oncotarget. Impact Journals, LLC. 2018; 9 (6): 7204.
Cherkasov A, et al. Use of artificial intelligence in the design of small peptide antibiotics effective against a broad spectrum of highly antibiotic-resistant superbugs. ACS Chem Biol 2009; 4:65.
Chia-Jen Shih Yueh-Lin Wu, et al., Association between Use of Oral Anti-Diabetic Drugs and the Risk of Sepsis: A Nested Case-Control Study. Sci Rep. 2015; 5:15260.
Christina Nedeva et. al., Sepsis: Inflammation Is a Necessary Evil. Frontiers in Cell and Developmental Biology, 2019; 7 (108).
Claire N. et al., Prevalence, Clinical Characteristics, and Outcomes of Sepsis Caused by Severe Acute Respiratory Syndrome Coronavirus 2 Versus Other Pathogens in Hospitalized Patients With COVID-19. Crit Care Explor. 2022; 4 (5): e0703.
Dalhoff A. Global Fluoroquinolone Resistance Epidemiology and Implications for Clinical Use. Interdisciplinary Perspectives on Infectious Diseases. 2012.
Danai P A, et al., Seasonal variation in the epidemiology of sepsis. Crit Care Med 2007; 35:410.
Dejager L, et al., Cecal ligation and puncture: the gold standard model for polymicrobial sepsis? Trends Microbiol, 2011; 19:198.
Dellinger R P, et al., Surviving Sepsis Campaign: international guidelines for management of severe sepsis and septic shock: 2008. Crit Care Med 2008; 36:296.
Di Giandomenico A et al., The “genomic storm” induced by bacterial endotoxin is calmed by a nuclear transport modifier that attenuates localized and systemic inflammation. PLoS ONE 2014; 9: e110183.
Donghong Yan, et al., Anti-IL-10 Therapeutic Strategy Using the Immunomodulator AS101 in Protecting Mice from Sepsis-Induced Death: Dependence on Timing of Immunomodulating Intervention. J Immunol 2002; 169:384.
Du B, et al., Extended-spectrum beta-lactamase-producing Escherichia coli and Klebsiella pneumoniae bloodstream infection: risk factors and clinical outcome. Intensive Care Med. 2002. PMID: 12447513.
E. Choy, Understanding the dynamics: pathways involved in the pathogenesis of rheumatoid arthritis, Rheumatology, 51, supplement 2012; 5:3.
E. Jones, et al., Procalcitonin test in the diagnosis of bacteremia: a metaanalysis, Annals of Emergency Medicine, 2007; 50, 1, 34.
E. L. Tsalik, et al., Discriminative value of inflammatory biomarkers for suspected sepsis, Journal of Emergency Medicine, 2012; 43, 1, 97.
Eicher S D, McMunn K A, Hammon H M, Donkin S S. Toll-like receptors 2 and 4, and acute phase cytokine gene expression in dexamethasone and growth hormone treated dairy calves. Vet Immunol Immunopathol 2004; 98:115.
Engel, et al., An analysis of interleukin-8, interleukin-6 and C-reactive protein serum concentrations to predict fever, gram-negative bacteremia and complicated infection in neutropenic cancer patients, Infection, 1998; 26, 4, 213.
Eun Jung Park et al., Dehydrocostuslactone inhibits LPS-induced inflammation by p38MAPK-dependent induction of hemeoxygenase-1 in vitro and improves survival of mice in CLP-induced sepsis in vivo. International Immunopharmacology 2014; 22:332.
European Centre for Disease Prevention and Control. Point prevalence survey of healthcare-associated infections and antimicrobial use in European acute care hospitals. ECDC, Stockholm; 2013.
European Commission Directive on the Classification, Packaging and Labelling of Dangerous Substances in the European Union, January 1997. Directive 67/548/EEC; Annex V. Revision. European Commission, 1997.
Evelien Gerits et al., Elucidation of the Mode of Action of a New Antibacterial Compound Active against Staphylococcus aureus and Pseudomonas aeruginosa, PLoS One 2016; 11.
Fang Liu et. al., Acetate Ringer's solution versus 0.9% saline for septic patients: study protocol for a multi-center parallel controlled trial. Trials (2021) 22:89.
Fang-Yin Kuo et. al., Affinity capture using peptide-functionalized magnetic nanoparticles to target Staphylococcus aureus Nanoscale, 2016; 8 (17).
FDA Drug Shortages. Accessed: August 2016.
Fletcher T C, et al., Extended antiinflammatory action of a degradation-resistant mutant of cell-penetrating suppressor of cytokine signaling 3. The Journal of Biological Chemistry. 2010; 285:18727-36.
Florea Lupu et al., Crosstalk between the coagulation and complement systems in sepsis. Thrombosis Research 133 (2014) S28.
Fourrier, et al., Septic shock, multiple organ failure, and disseminated intravascular coagulation. Compared patterns of antithrombin III, protein C, and protein S deficiencies. Chest 1992; 101:816.
G. Trinchieri, Interleukin-12 and the regulation of innate resistance and adaptive immunity, Nature Reviews Immunology, 2003; 3, no. 2, 133.
Gasnik L B, et al. Risk factors for and impact of infection or colonization with aztreonam-resistant Pseudomonas aeruginosa. Infect Control Hosp Epidemiol. 2007 October; 28:1175.
Gil, M, et al., Naringin decreases TNF-a and HMGB1 release from LPS-stimulated macrophages and improves survival in a CLP-induced sepsis in mice. PLoS One, 2016; 11: e0164186.
Global Data. Sepsis—Opportunity Analysis and Forecasts to 2021.
Grace Y. Song, et al., What is the role of interleukin 10 in polymicrobial sepsis: Anti-inflammatory agent or immunosuppressant? Surgery 1999; 126:378.
Guidance Document on Using In vitro Data to Estimate In vivo Starting Doses for Acute Toxicity: Interagency Coordinating Committee on the Validation of Alternative Methods (ICCVAM), 2001. Report of the International Workshop on In vitro Methods for Assessing Acute Systemic Toxicity. NIH Publication No. 01-4499/4500. National Institute of Environmental Health Sciences, Research Triangle Park, N C.
Guido Marcello M et al., In vitro diagnosis of sepsis: a review. Pathology and Laboratory Medicine International, 2016; 8.
Gums J G. Assessing the impact of antimicrobial resistance. Am J Health Syst Pharm; 2002; 59 (8 Suppl.3): S4.
Gupta A K, et al., Analgesic and Anti-Inflammatory Properties of Gelsolin in Acetic Acid Induced Writhing, Tail Immersion and Carrageenan Induced Paw Edema in Mice. PLoS ONE, 2015; 10 (8): e0135558.
Gustavo Matute-Bello, et al., An Official American Thoracic Society Workshop Report: Features and Measurements of Experimental Acute Lung Injury in Animals. Am J Respir Cell Mol Biol. 2011; 44. 725.
Hall-Stoodley et al., Bacterial biofilms: from the natural environment to infectious diseases. Nat. Rev. Microbiol., 2004; 2, 95.
Hancock R E, et al., Role of membranes in the activities of antimicrobial cationic peptides. FEMS Microbiol Lett 2002; 206:143.
Hancock R E, et al., Antimicrobial and host-defense peptides as new anti-infective therapeutic strategies. Nat Biotechnol 2006; 24:1551.
Hancock R E W, et al., The role of cationic antimicrobial peptides in innate host defences. Trends in Microbiology 2000; 8:402.
Hellman J, et al, Part III: minimum quality threshold in preclinical sepsis studies (MQTiPSS) for fluid resuscitation and antimicrobial therapy endpoints. Shock, 2019; 51:33.
Heming N, et al., Emerging drugs for the treatment of sepsis. Expert Opin Emerg Drugs. 2016.
Henrique G, Colac, o et al., Tetracycline Antibiotics Induce Host-Dependent Disease Tolerance to Infection. Immunity, 2020; 53, 1.
Hina Chaudhry et al., role of Cytokines as a Double-edged Sword in Sepsis. In vivo. 2013; 27 (6): 669.
Hojo, K. et al., Bacterial interactions in dental biofilm development. J. Dent. Res., 2009; 88, 982.
Hojo, K. Park et al., The Role of Antimicrobial Peptides in Preventing Multidrug-Resistant Bacterial Infections and Biofilm Formation, Int. J. Mol. Sci., 2009; 12, 5971.
Hong Z, et al. Chloroquine protects mice from challenge with CpG ODN and LPS by decreasing proinflammatory cytokine release. Int Immunopharmacol 2004; 4:223.
Hong Zhu. et. al., Efficacy and safety of antimicrobial de-escalation of treatment for sepsis. Medicine (Baltimore). 2020; 99 (49): e23385.
Hosny, Mohamed et. al., Impact of oral omega-3 fatty acids supplementation in early sepsis on clinical outcome and immunomodulation. The Egyptian Journal of Critical Care Medicine, 2013; 1 (3); 119
Hotchkiss, R. S., G. Monneret, and D. Payen. Sepsis-induced immunosuppression: from cellular dysfunctions to immunotherapy. Nat. Rev. Immunol. 2013; 13:862.
Hubbard, et al., Cecal ligation and puncture. Shock 2005; 24 (Suppl. 1): 52.
Huh K. et al., Risk factors and treatment outcomes of bloodstream infection caused by extended-spectrum cephalosporin-resistant Enterobacter species in adults with cancer. Diagn Microbiol Infect Dis. 2014 February; 78 (2): 172.
Iberio S A, et al., antimicrobial activity against oral pathogens and immunomodulatory effects and toxicity of geopropolis produced by the stingless bee Melipona fasciculata Smith. BMC Complement Altern Med. 2011; 11:108.
Ignacio Rubio, et al., Current gaps in sepsis immunology: new opportunities for translational research, The Lancet Infectious Diseases.2019, 19 (12), 422.
Syed Murthuza, et al., In vitro and in vivo evaluation of anti-inflammatory potency of Mesua ferrea, Saraca asoca, Viscum album & Anthocephalus cadamba in murine macrophages raw 264.7 cell lines and Wistar albino rats Beni-Suef University Journal of Basic and Applied Sciences 2018; 7,719.
Isabella F. S. Figueiredo, et al., Cinnamaldehyde Increases the Survival of Mice Submitted to Sepsis Induced by Extraintestinal Pathogenic Escherichia coli. Antibiotics 2022, 11, 364.
Itaynara L. Dutra, et al., Pic-Producing Escherichia coli Induces High Production of Proinflammatory Mediators by the Host Leading to Death by Sepsis. Int J Mol Sci. 2020; 21 (6): 2068.
J. Cohen, The immunopathogenesis of sepsis, Nature, 2002; 420, 6917, 885.
J. Deen, L. et al., Community-acquired bacterial bloodstream infections in developing countries in south and southeast Asia: a systematic review, The Lancet Infectious Diseases, 2012; 12, 6:480.
J. Hawiger et al., New paradigms in sepsis: from prevention to protection of failing microcirculation. Journal of Thrombosis and Haemostasis, 2015; 13:1743.
J. M. Rubio-Perez et al., A review: inflammatory process in Alzheimer's disease, role of cytokines, The Scientific World Journal, 2012, Article I D 756357.
J. Cohen. Non-antibiotic strategies for sepsis. Clinical Microbiology and Infection, 2009; 15, 4, 302.
James D. Faix. Biomarkers of sepsis. Critical Reviews in Clinical Laboratory. Sciences. 2013; 50 (1) IDrugs 2007; 10 (4): 264.
Jean A Nemzek et al., Modeling Sepsis in the Laboratory: Merging Sound Science with Animal Well-Being. Comp Med. 2008; 58 (2): 120.
Jean-Marc Cavaillon, et al., Sepsis therapies: learning from 30 years of failure of translational research to propose new leads. EMBO Mol Med, 2020; e10128.
Jeremy Weinberger et al., A Critical Analysis of the Literature on Time-to-Antibiotics in Suspected Sepsis. The Journal of Infectious Diseases, 2020; 222 (S2): S110-8.
Jesse M. Jaynes et al., “GENOPEP”, a Topical Cream in the Treatment of Burn Wounds. ACS symposium series e books, 2012.
Jiang Z, et al. A synthetic peptide derived from bactericidal/permeability-increasing protein neutralizes endotoxin in vitro and in vivo. Int Immunopharmacol 2004; 4:527.
Jinbao Li et al., Neutralisation of Peritoneal IL-17A Markedly Improves the Prognosis of Severe Septic Mice by Decreasing Neutrophil Infiltration and Proinflammatory Cytokines. Article in PLoS ONE, 2012.
Jo D, et al., Hawiger J. Intracellular protein therapy with SOCS3 inhibits inflammation and apoptosis. Nat Med 2005; 11:892.
Jonathan Cohen et al., The immunopathogenesis of sepsis. Nature 2002; 420 (19/26).
Jou J. Jawien, New insights into immunological aspects of atherosclerosis, Polskie ArchiwumMedycyny Wewnetrznej, 2008; 118 (3) 127-131.
Julia Suhorutsenko et. al., Cell-Penetrating Peptides, Pep Fects, Show No Evidence of Toxicity and Immunogenicity In vitro and In vivo. Bioconjugate Chem. 2011; 22, 2255.
K A Kirkebøen et al., The role of nitric oxide in sepsis—an overview. Acta Anaesthesiol Sc, 1999; 43 (3): 275.
K. Kofoed, et al., Use of plasma C-reactive protein, procalcitonin, neutrophils, macrophage migration inhibitory factor, soluble urokinase-type plasminogen activator receptor, and soluble triggering receptor expressed on myeloid cells-1 in combination to diagnose infections: a prospective study, Critical Care, 2007; 11.
K. Reinhart, M. et al., new Approaches to sepsis: molecular diagnostics and biomarkers, journal of Clinical Microbiology, 2012; 25, 4, 609.
Kaneider N C, et al., Role reversal for the receptor PARI in sepsis-induced vascular damage. Nat Immunol 2007; 8:1303.
Katsuhiro Osajima et al., Safety Evaluation of a Peptide Product Derived from Sardine Protein Hydrolysates (Valtyron). Journal of Toxicology, 2009; 28 (5) 341.
Kengo Tomita & Yuna Saito et. al., Vascular endothelial growth factor contributes to lung vascular hyperpermeability in sepsis-associated acute lung injury, Naunyn-Schmiedeberg's Archives of Pharmacology. 2020; 393:2365.
Kent Doi, M. D. et al., pre-existing Renal Disease Promotes Sepsis-induced Acute Kidney Injury and Worsens Sepsis Outcome via Multiple Pathways. Kidney Int. 2008; 74 (8): 1017
Key Role for VEGF in Onset of Sepsis, by Beth Israel Lahey Health. Journal of Experimental Medicine (2006).
Kiichiro Yano, et al., Vascular endothelial growth factor is an important determinant of sepsis morbidity and mortality. J Exp Med.2006; 203 (6): 1447.
Kim, Y. K., et al., Tussilagone inhibits the inflammatory response and improves survival in CLP-Induced septic mice. Int. J. Mol. Sci. 2017; 18:2744.
Kishimoto, T. IL-6: from its discovery to clinical applications. Int. Immunol. 2010; 22: 347.
Konstantin Tsoyi, et al., HO-1 inducers and CO-releasing molecule inhibit LPS-induced HMGB 1 release in vitro and improves survival of mice in LPS- and CLP-induced sepsis model in vivo. Molecular Pharmacology, 2009.
Kristina E Rudd et. al., Global, regional, and national sepsis incidence and mortality, 1990-2017: analysis for the Global Burden of Disease Study. Lancet 2020; 395:200.
Kyung-jun jang et al., Anti-inflammatory potential of total saponins derived from the roots of Panax ginseng in lipopolysaccharide-activated RAW 264.7 macrophages, Experimental and therapeutic medicine 2016; 11:1109.
L. Simon, et al., “Serum procalcitonin and C-reactive protein levels as markers of bacterial infection: a systematic review and meta-analysis,” Clinical Infectious Diseases, 2004; 39, 2, 206.
Labby K J. et al., Strategies to overcome the action of aminoglycoside-modifying enzymes for treating resistant bacterial infections. Future Med Chem. 2013 July; 5 (11): 1285.
Lars Steinstraesser et al., Host defense peptides as effector molecules of the innate immune response: a sledgehammer for drug resistance? Int J Mol Sci.2009; 10 (9): 395.
Levi, M, et al., Disseminated intravascular coagulation. N. Engl. J. Med. 1999; 341: 586.
Levi, M, et al., Inflammation and coagulation. Crit. Care Med. 2010; 38 (2 Suppl.): 26.
Liao J C, et al., Anti-inflammatory activities of Cinnamomum cassia constituents in vitro and in vivo. Evid Based Complement Alternat Med 2012:429320, 2012.
Libert C, et al., Part II: minimum quality threshold in pre-clinical sepsis studies (MQTiPSS) for types of infections and organ dysfunction endpoints. Shock2019; 51:23
Liu C et al., Clinical practice guidelines by the infectious diseases society of America for the treatment of methicillin-resistant Staphylococcus aureus infections in adults and children. Clin Infect Dis. 2011; 52:18.
Liu Y, et al., Scutellarin suppresses NLRP3 inflammasome activation in macrophages and protects mice against bacterial Sepsis. Front Pharmacol 2017; 8:975.
London N R, et al., Targeting Robo4-dependent Slit signaling to survive the cytokine storm in sepsis and influenza. Sci Transl Med. 2010; 2: 23ra19.
Lowy F D. Staphylococcus aureus infections. N Engl J Med. 1998; 8:520.
Lucineia Gainski et al., NLRP3 Activation Contributes to Acute Brain Damage Leading to Memory Impairment in Sepsis-Surviving Rats. Molecular Neurobiology. Molecular Neurobiology, 2020.
M. Barati, et al., Comparison of WBC, ESR, CRP and PCT serum levels in septic and non-septic burn cases, Burns, 2008; 34, 6,770.
M. H. V. Van Regenmor et al., Antigenicity and Immunogenicity of Synthetic Peptides. Biologicals 2001; 29, 209.
M. Limper, et al., The diagnostic role of Procalcitonin and other biomarkers in discriminating infectious from non-infectious fever, Journal of Infection, 2010; 60, 6, 409-416.
M. Rincon, Interleukin-6: from an inflammatory marker to a target for inflammatory diseases, Trends in Immunology, 2012; 33, 11:571.
M. Schouten, et al., Poll, Inflammation, endothelium, and coagulation in sepsis. J Leukoc Biol. 2008; 83 (3): 536.
M. Seitz, et al., Interleukin-10 differentially regulates cytokine inhibitor and chemokine release from blood mononuclear cells and fibroblasts, European Journal of Immunology, 1995; 25, no. 4, 1129.
M. W. Petersen et al., The anti-inflammatory effect of exercise, Journal of Applied Physiology, 2005; 98 (4): 1154.
Magudumana O, et al. Serial interleukin 6 measurements in the early diagnosis of neonatal sepsis. J Trop Pediatr. 2000; 46:267.
Mah, T. F. et al., Mechanisms of biofilm resistance to antimicrobial agents. Trends in microbiology, 2001; 9, 34.
Manasi Nandi, Rethinking animal models of sepsis-working towards improved clinical translation whilst integrating the 3Rs. Clinical Science, 2020; 134 1715.
Marcin F. Osuchowski et al., Minimum quality threshold in pre-clinical sepsis studies (mqtipss): an international expert consensus initiative for improvement of animal modeling in sepsis: International Expert Consensus for Pre-Clinical Sepsis Studies. shock, 2018; 50 (4) 377.
María Luisa Martínez et. al., An approach to antibiotic treatment in patients with sepsis J Thorac Dis 2020; 12 (3): 1007.
Mateus Curioa, et al., Acute effect of Copaifera reticulata Ducke copaiba oil in rats tested in the elevated plus-maze: an ethological analysis. JPP 2009, 61:1105.
Matsukawa, A., et al., Aberrant inflammation and lethality to septic peritonitis in mice lacking STAT3 in macrophages and neutrophils. J. Immunol. 2003; 171:6198.
McCoy S L, et al., Activation of RAW264.7 macrophages by bacterial DNA and lipopolysaccharide increases cell surface DNA binding and internalization. J Biol Chem 2004; 279:17217.
Min Jee Kim, Aged Black Garlic Exerts Anti-Inflammatory Effects by Decreasing NO and Proinflammatory Cytokine Production with Less Cytoxicity in LPS-Stimulated RAW 264.7 Macrophages and LPS-Induced Septicemia Mice., J Med Food, 2014; 17 (10), 1057.
Mohamed F. et al, Evaluation of short synthetic antimicrobial peptides for treatment of drug-resistant and intracellular Staphylococcus aureus, Scientific Reports 2016; 6:29707. DOI: 10.1038/srep29707, 1.
Mohammad, H. et al., antimicrobial peptides and peptidomimetics-potent therapeutic allies for staphylococcal infections. Current pharmaceutical design 2015; 21, 2073.
Monroe D. Looking for chinks in the armor of bacterial biofilms. PLoS Biol., 2007; 5, e307.
Mookherjee N, et al., Cationic host defence peptides: Innate immune regulatory peptides as a novel approach for treating infections. Cellular and Molecular Life Sciences 2007; 64:922.
Mounyr Balouiri et al., Methods for in vitro evaluating antimicrobial activity: A review, Journal of Pharmaceutical Analysis, 2016; 6:71.
N. Chaudhary, et al., Significance of interleukin-6 (IL-6) and C-reactive protein (CRP) in children and young adults with febrile neutropenia during chemotherapy for cancer: a prospective study, Journal of Pediatric Hematology/Oncology, 2012; 34, 8, 617.
Neal R. Chamberlain, lecture on Sepsis and Septic Shock, 2018. Available at: https://www.atsu.edu/faculty/chamberlain/website/lectures/lecture/sepsis2007.htm.
NEWS AND OPINIONS 2022; Sweden: Pernilla's 8-day old daughter died from sepsis—caused by resistant bacteria Klebsiella.
Nguyen H B et al., Severe sepsis and septic shock: review of the literature and emergency department management guidelines. Ann Emerg Med. 2006 July; 48 (1): 28-54.
Nickel, J. C. et al., Bacterial biofilms: influence on the pathogenesis, diagnosis, and treatment of urinary tract infections. J. Antimicrob. Chemother. 1994, 33, 31.
Nicole J. Afacan, Amy T. Y. Yeung, Olga M. Pena and Robert E. W. Hancock. Therapeutic Potential of Host Defense Peptides in Antibiotic-resistant Infections. Current Pharmaceutical Design, 2012, 18, 807-819 807.
Niels C Riedemann et. al., Novel strategies for the treatment of sepsis. Nat Med, 2003; 9 (5): 517.
Nijnik A, et al., Synthetic cationic peptide IDR-1002 provides protection against bacterial infections through chemokine induction and enhanced leukocyte recruitment. J Immunol. 184:2539.
OECD Guidelines for the Testing of Chemicals, 1987. OECD 401. Acute Oral Toxicity. Organisation for Economic Cooperation and Development, Paris.
OECD Guidelines for the Testing of Chemicals, 2001. OECD 420. Acute Oral Toxicity—Fixed Dose Procedure. Organisation for Economic Cooperation and Development, Paris.
OECD Guidelines for the Testing of Chemicals, 2001. OECD 423. Acute Oral Toxicity—Acute Toxic Class Method. Organisation for Economic Cooperation and Development, Paris.
OECD Guidelines for the Testing of Chemicals, 2001. OECD 425. Acute Oral Toxicity—Modified Up and Down Procedure. Organisation for Economic Cooperation and Development, Paris.
Olufunmiso O Olajuyigbe et al., In vitro Synergy and Time-kill Assessment of Interaction between Kanamycin and Metronidazole against Resistant Bacteria, Tropical Journal of Pharmaceutical Research; 2105; 14 (5): 837.
Opal S M. New perspectives on immunomodulatory therapy for bacteraemia and sepsis. International Journal of Antimicrobial Agents 2010; 36:70.
Oren Z, et al., Structure and organization of the human antimicrobial peptide LL-37 in phospholipid membranes: relevance to the molecular basis for its non-cell-selective activity. Biochem J. 1999; 341 (Pt 3): 501.
Osmar N. Silva et al., Clavanin A Improves Outcome of Complications from Different Bacterial Infections. Antimicrob Agents Chemother. 2015; 59 (3): 1620-1626.
P. Puccetti, et al., Effects of IL-12 and IL-23 on antigen-presenting cells at the interface between innate and adaptive immunity, Critical Reviews in Immunology, 2002; 22, 5-6:373-390.
P. A. Botham. Acute systemic toxicity-prospects for tiered testing strategies Toxicology, in vitro 18 (2004) 227-230.
Peters N K et al., The research agenda of the National Institute of Allergy and Infectious Diseases for Antimicrobial Resistance. J Infect Dis, 2008; 197 (8): 1087.
Pradhan, S., et al., Evaluation of phytochemical, antimicrobial and time-killing assay of Camellia species. Vegetos 2020; 33, 759.
Qiao H, et al., The adaptor CRADD/RAIDD controls activation of endothelial cells by proinflammatory stimuli. J Biol Chem 2014; 289:21973-83.
R. Berner, et al., “Plasma levels and gene expression of granulocyte colony-stimulating factor, tumor necrosis factor-α, interleukin (IL)-1β, IL-6, IL-8, and soluble intercellular adhesion molecule-1 in neonatal early onset sepsis,” Pediatric Research, 1998; 44, 4, 469.
Rangel-Frausto M S et al., The natural history of the systemic inflammatory response syndrome (SIRS). A prospective study. JAMA 1995; 273:117.
Rashad A. et al., Cytotoxicity and Anti-Inflammatory Activity of Methylsulfanyl-triazoloquinazolines, Molecules. 2013; 18 (2): 1434.
Rauch S, et al., Vaccine protection of leukopenic mice against Staphylococcus aureus bloodstream infection. Infect Immun 2014; 82:4889-98.
Reinhart K, et al., Recognizing sepsis as a global health priority—a WHO resolution. New Engl J Med 2017; 377:414-17.
Renckens R, et al., Endogenous tissue-type plasminogen activator is protective during Escherichia coli-induced abdominal sepsis in mice. J Immunol. 2006 Jul. 15; 177 (2): 1189.
Rhodes A, et al. Surviving sepsis campaign: International Guidelines for Management of Sepsis and Septic Shock: 2016. Intensive Care Med 2017; 43:304.
Riedemann, et al., Protective effects of IL-6 blockade in sepsis are linked to reduced C5a receptor expression. J. Immunol. 2003; 170:503.
Rina Barouch et al., Nerve growth factor regulates TNF-a production in mouse macrophages via MAP kinase activation. Journal of Leukocyte Biology 2001; 69.
Rittirsch D, et al., Immuno design of experimental sepsis by cecal ligation and puncture. Nat Protoc, 2009; 4:31.
Robert G. Strickley, Solubilizing Excipients in Oral and Injectable Formulations: Review article. Pharmaceutical Research, 2004; 21, 2.
Rodrigo de Almeida et al., Evaluation of the immunogenicity and in vivo toxicity of the antimicrobial peptide P34. International Journal of Pharmaceutics 42, 2011; 94.
S Sriskandan, et. al., Early cytokines nitric oxide. The Journal of Pathology, 2008; 214, 2 21 (1-223).
S. Bhattacharya, Blood culture in India: a proposal for a national programme for early detection of sepsis,” Indian Journal of Medical Microbiology, 2005; 23, 4. 220-226.
S. Calfee, et al., Plasma interleukin-8 is not an effective risk stratification tool for adults with vasopressor dependent septic shock, Critical Care Medicine, 2010; 38, no. 6, pp. 1436.
S. Gibot, N. C. et al., Combination biomarkers to diagnose sepsis in the critically ill patient, The American Journal of Respiratory and Critical Care Medicine, 2012; 186, 65.
S. Kumar and M. Rizvi, “Serum tumor necrosis factor alpha and C-reactive protein in pediatric patients with sepsis and its correlation with microbiologic findings,” Indian Journal of Pathology and Microbiology, 2010; 53, 3, 494.
S. Q. Latifi, et al., Interleukin-10 controls the onset of irreversible septic shock, Infection and Immunity, 2002; 70, 8:4441.
Safiah Mai, et al., Book chapter: Experimental Sepsis Models, 2020 on behalf of the Canadian Critical Care Translational Biology Group (CCCTBG).
Sari Karlsson et al., Vascular endothelial growth factor in severe sepsis and septic shock. Anesth Analg. 2008; 106 (6): 1820.
Sartelli M, et al., Raising concerns about the Sepsis-3 definitions. World J Emerg Surg 2018; 13:6.
Savitri Kibe et al., Diagnostic, and prognostic biomarkers of sepsis in critical care. J. Antimicrob Chemother 2011; 66 Suppl 2:33-40.
SCHEDULE Y, DCGI-CDSCO document The Drugs & Cosmetics Act, 1940.
Schmalz G, et al., Toll-like receptors, LPS, and dental monomers. Adv Dent Res 2011; 23:302-306.
Schulte, W., et al., Cytokines in sepsis: potent immunoregulators and potential therapeutic targets—an updated view. Mediators Inflamm. 2013:165974.
Schwab U et al., In vitro activities of designed antimicrobial peptides against multidrug resistant cystic fibrosis pathogens. Antimicrob Agents Chemother; 1999; 43:1435.
Schwartz S, et al., Pharmacokinetics, disposition and metabolism of 546C88 (L-N(G)-methylarginine hydrochloride) in rat and dog. Xenobiotica. 1997; 27 (12): 1259.
Sepsis Alliance. Definition of sepsis.
Available at: http://www.sepsisalliance.org/sepsis/definition. Accessed Jun. 4, 2015.
Şerife Kurul, et al., Association of inflammatory biomarkers with subsequent clinical course in suspected late onset sepsis in preterm neonates. Crit Care. 2021; 25:12.
Shankar-Hari M, et al., Do we need a new definition of sepsis? Intensive Care Med. 2015; 41:909.
Sharawy N. Vasoplegia in septic shock: do we really fight the right enemy? J Crit Care 2014; 29:83-7.
Sharma Rakesh, et al., Anti-Inflammatory Activity of Moringa Oleifera Leaf and Pod Extracts Against Carrageenan Induced Paw Edema in Albino Mice. Pharmacology online 2011; 1:140.
Shayne C. Gad, et al., Nonclinical Vehicle Use in Studies by Multiple Routes in Multiple Species. International Journal of Toxicology, 2006; 25:499.
Shrum B, et al, A robust scoring system to evaluate sepsis severity in an animal model. BMC Res Notes 2014; 7:233.
Silva, O. N., et al. An anti-infective synthetic peptide with dual antimicrobial and immunomodulatory activities. Scientific Reports, 2016; 6 1): 35465.
Singleton K, et al., Distance of cecum ligated influences mortality, tumor necrosis factor-alpha and interleukin-6 expression following cecal ligation and puncture in the rat. Eur Surg Res, 2003; 35:486.
Skibsted S, et al., NI. Biomarkers of endothelial cell activation in early sepsis. Shock. 2013; 39:427.
Sligl W I, et al. Nosocomial Gram-negative bacteremia in intensive care: epidemiology, antimicrobial susceptibilities, and outcomes. Int J Infect Dis. 2015 August; 37:129.
Soerya Dewi Marliyana et al., Time-Kill Assay of 4-Hydroxypanduratin A Isolated from Kaempferia pandurata Against Foodborne Pathogens. Molekul, 2017; Vol. 12. No. 2:166.
Solomkin J S, et al. Diagnosis and management of complicated intra-abdominal infection in adults and children: guidelines by the Surgical Infection Society and the Infectious Diseases Society of America. Clin Infect Dis 2010; 11:79.
Srijan Tandukar, M D et al., Continuous Renal Replacement Therapy Who, When, Why, and How. Chest. 2019; 155 (3): 626.
Stefan Wirtz et al., Protection from lethal septic peritonitis by neutralizing the biological function of interleukin 27, the journal of experimental medicine. 2006; 203, No. 82, 1875.
Stephen B. Pruett, et al., Innate Immunity and Inflammation in Sepsis: Mechanisms of Suppressed Host Resistance in Mice Treated with Ethanol in a Binge-Drinking Model, Toxicological Sciences, 2010; 117, 2, 314.
Stevens R D, et al., A framework for diagnosing and classifying intensive care unit-acquired weakness. Crit Care Med. 2009 October; 37 (10 Suppl): S299-308.
Stewart, P. S. et al., Antibiotic resistance of bacteria in biofilms. Lancet, 2001; 358, 135.
Sudhir Verma, Laboratory Animal Models to Mimic Human Sepsis: A Review. Research & Reviews: Journal of Zoological Sciences RRJZS, 2016; 4 (2) 34.
Sugitharini, V et al., Inflammatory mediators of systemic inflammation in neonatal sepsis. Inflamm. Res. 2013; 62, 1025.
Sutthiwan Thammawat et al., Time-kill profiles and cell-surface morphological effects of crude Polycephalomyces nipponicus Cod-MK1201 mycelial extract against antibiotic-sensitive and -resistant Staphylococcus aureus, Tropical Journal of Pharmaceutical Research; 2015; 16 (2): 407.
Thomas Marichal et al., Role of Neutrophils in inflammatory diseases. Frontiers in Immunology, 2020; 11.
Timothy W. Evans, et. al., Mechanisms of Organ Dysfunction in Critical Illness Springer Science & Business Media, 21 Jan. 2002—Medical—410 pages.
Tjabringaa G S, et al., Human Cathelicidin LL-37 Is a Chemoattractant for Eosinophils and Neutrophils That Acts via Formyl-Peptide Receptors. Int Arch Allergy Immunol 2006; 140:103-12.
Tobias Schuerholz, et al. The anti-inflammatory effect of the synthetic antimicrobial peptide 19-2.5 in a murine sepsis model: a prospective randomized study. Critical Care 2013, 17: R3.
V. Kumar et al., Toll-like receptors in sepsis-associated cytokine storm and their endogenous negative regulators as future immunomodulatory targets. International Immunopharmacology.2020; 89:107087.
Veach R A, et al., Lethality in a murine model of pulmonary anthrax is reduced by combining nuclear transport modifier with antimicrobial therapy. PLoS ONE 2012; 7: e30527.
Vinsonneau C, et al., Continuous venovenous hemodiafiltration versus intermittent haemodialysis for acute renal failure in patients with multiple-organ dysfunction syndrome: a multicentre randomised trial. Lancet. 2006; 368 (9533): 379.
Wang and Wang et al., Identification of risk factors for in-hospital death of COVID-19 pneumonia—lesions from the early outbreak. BMC Infectious Diseases 2021; 21:113.
Wang J, et al. The antimalarial artemisinin synergizes with antibiotics to protect against lethal live Escherichia coli challenge by decreasing proinflammatory cytokine release. Antimicrob Agents Chemother 2006; 50:2420-7.
Wegiel B, et al, Piperine metabolically regulates peritoneal resident macrophages to potentiate their functions against bacterial infection. Oncotarget 2015; 6:32468-32483.
Wendy E. Walker (ed.), Sepsis: Methods and Protocols, Methods in Molecular Biology, 2021; 2321.
WHO Coronavirus Disease (COVID-19) Dashboard. Available from: https://covid19.who.int. Accessed 24 Oct. 2020.
Wibke Schulte et al., Cytokines in Sepsis: Potent Immunoregulators and Potential Therapeutic Targets—An Updated View. Mediators Inflamm. 2013; 2013:165974.
William C. Aird et al., The role of the endothelium in severe sepsis and multiple organ dysfunction syndrome. Blood, 2003; 101 (10), 3765.
Wilson S E, et al., Comparative costs of ertapenem and cefotetan as prophylaxis for elective colorectal surgery. Surg Infect. 2008; 9:349.
Wong G, et al., Backs against the wall: novel and existing strategies used during the 2014-2015 Ebola virus outbreak. Clin Microbiol Rev 2015; 28:593.
World Health Organization. Antimicrobial resistance: global report on surveillance. WHO, Geneva; 2014.
Wu M, Maier E, et al., Mechanism of interaction of different classes of cationic antimicrobial peptides with planar bilayers and with the cytoplasmic membrane of Escherichia coli. Biochemistry. 1999; 38:7235.
Xia, Z., et al., Opposing effects of ERK and JNK-p38 MAP kinases on apoptosis. Science. 1995; 270, 1326.
Xiang Zhou, et. al., Rules of anti-infection therapy for sepsis and septic shock, Chinese Medical Journal 2019; 132 (5).
Xiaodan Yang, et al., Preclinical Pharmacokinetics, Biodistribution, and Acute Toxicity Evaluation of Caerin 1.9 Peptide in Sprague Dawley Rats. Evidence-Based Complementary and Alternative Medicine 2022, Article ID 9869293.
Xiaofeng Niu et al., Isofraxidin exhibited anti-inflammatory effects in vivo and inhibited TNF-α production in LPS-induced mouse peritoneal macrophages in vitro via the MAPK pathway International Immunopharmacology 14, 2012; 164-171.
Xiaofeng Niu, et al., Esculin exhibited anti-inflammatory activities in vivo and regulated TNF-α and IL-6 production in LPS-stimulated mouse peritoneal macrophages in vitro through MAPK pathway, International Immunopharmacology, 2015.
Yali Zhang, et al., Anti-inflammatory effects of novel curcumin analogs in experimental acute lung injury Respiratory Research (2015) 16:43.
Yang D, et al., The role of mammalian antimicrobial peptides and proteins in awakening of innate host defenses and adaptive immunity. Cellular and Molecular Life Sciences 2001; 58:978.
Yea-Chen Liu, et al., Extract and Its Bioactive Compounds Exert Anti-inflammatory Activities and VEGF Production Through M2-Macrophagic Polarization and STAT6 Activation. J Med Food 2018, 1.
Yona Kalechman, et al., Immunomodulator AS101 in Protecting Mic from Sepsis-Induced Death: Dependence on Timing of Immunomodulating Intervention, J Immunol 2002; 169:384.
Yoon Ju Chol, et al., Kinetics of proinflammatory cytokines after intraperitoneal injection of tribromoethanol and a tribromoethanol/xylazine combination in ICR mice. Lab Anim Res 2011: 27 (3), 197.
Yung-Yang Liu, et al., High-molecular-weight hyaluronan—a possible new treatment for sepsis-induced lung injury: a preclinical study in mechanically ventilated rats. Critical Care 2008, 12: R102.
Zhengwen Xiao, et al., Inflammatory mediators in intra-abdominal sepsis or injury—a scoping review. Critical Care, 2015; 19, (373).
Zingarelli B, et al, Part I: minimum quality threshold in preclinical sepsis studies (MQTiPSS) for study design and humane modeling endpoints. Shock 2019; 51:10.

1. A peptide with anti-inflammatory and anti-microbial activity, the peptide selected from the group consisting of one having an amino acid sequence of SEQ ID NO: 1 or variant thereof, and one having the chemical formula C42H66N10O8.

2. (canceled)

3. The peptide as claimed in claim 1, wherein the peptide inhibits Interferon gamma (IFN-y), thymus and activation regulated chemokine, Interleukin-8, thymic stromal lymphopoietin secretion, IL-β, IL-1β, IL-6, IL-10, IL-12, TNF-α, and CCL2 (MCP1).

4. A formulation comprising a peptide of SEQ ID NO: 1 or a variant thereof or a peptide of chemical formula C42H66N10O8 and one or more suitable pharmaceutically acceptable excipients for treating, preventing, alleviating and/or ameliorating inflammatory and/or microbial diseases or one or more symptoms associated thereof.

5. The formulation as claimed in claim 4, wherein said one or more suitable pharmaceutically acceptable excipients are selected from the group consisting of suitable carriers, starch, povidone, cellulose, lactose, magnesium stearate, microcrystalline cellulose, Poloxamer, Polysorbate 20, Sodium chloride, Sodium starch glycolate, anti-adherent, Calcium phosphate, stearic acid, colloidal silicon dioxide, colorants, sodium croscarmellose, diluents, crospovidone, glidant, mannitol, vehicles, disintegrant, swelling agent, antioxidant, buffer, bacteriostatic agent, emollient, emulsifier, plasticizer, penetration enhancer, preservative, cryoprotectant, neutralizer, fragrance additives, dispersants, surfactants, binders and lubricants.

6. The formulation as claimed in claim 4, wherein the peptide or variant thereof is present in an amount of 0.01 μg/mL to 1000 μg/mL.

7. The peptide as claimed in claim 1, for treatment, amelioration, alleviation and/or prevention of inflammatory and/or microbial disease and/or one or more symptoms associated thereof.

8. A method of synthesizing peptide of SEQ ID NO: 1 or a variant thereof comprising:
(a) preparing peptydil resin containing the peptide of SEQ ID NO: 1 (IS111) or variant thereof with resin;
(b) performing cleavage of the peptydil resin cocktail obtained in step (b) to obtain crude peptide; and
(c) optionally purifying the crude peptide obtained in step (b) to obtain pure peptide.

9. The method as claimed in claim 8, wherein the step a) for preparing peptydil resin containing the peptide of SEQ ID NO: 1 (IS111) or variant thereof with resin comprises the following steps of:
(i) placing resin in a reaction vessel and swelling with at least one suitable solvent;
(ii) washing the resin with at least one suitable solvent;
(iii) deprotecting by adding piperdine in at least one suitable solvent to the resin followed by stirring and draining;
(iv) obtaining a solution of Fluorenylmethyloxycarbonyl tyrosine (Fmoc Tyr) and Hydroxybenzotriazole (HOBT) in at least one suitable solvent followed by adding N,N′-Diisopropylcarbodiimide (DIC) to the solution and adding solution to the resin;
(v) mixing and checking for free amino acid by Ninhydrine test;
(vi) repeating steps (i) to (v) with next amino acid to obtain peptydil resin cocktail.

10. The method as claimed in claim 9, wherein said suitable solvent in step a) is Dimethylformamide (DMF), acetonitrile, methanol, methyl ethyl ketone, l-butanol, t-butanol, tert-butyl methyl ether, trimethylamine, toluene or any combination thereof, preferably DMF.

11. A method of synthesising peptide of SEQ ID NO: 1 or a variant thereof comprising:
(a) preparing peptydil resin containing the peptide of SEQ ID NO: 1 (IS111) or variant thereof with resin comprising the steps of
(i) placing resin in a reaction vessel of a synthesizer and swelling with Dimethylformamide (DMF);
(ii) washing the resin with DMF;
(iii) deprotecting by adding piperdine in DMF to the resin followed by stirring and draining;
(iv) obtaining a solution of Fluorenylmethyloxycarbonyl tyrosine (Fmoc Tyr) and Hydroxybenzotriazole (HOBT) in DMF followed by adding N,N′-Diisopropylcarbodiimide (DIC) to the solution and adding solution to the resin;
(v) mixing and checking for free amino acid by Ninhydrine test;
(vi) repeating steps (i) to (v) with next amino acid to obtain peptydil resin cocktail;
(b) performing cleavage of the peptydil resin cocktail obtained in step (b) to obtain crude peptide; and
(c) optionally purifying the crude peptide obtained in step (b) to obtain pure peptide.

12. A method of treating a subject infected with inflammatory or microbial diseases for treating, preventing, alleviating and/or ameliorating said inflammatory or microbial diseases or one or more symptoms associated thereof comprising administering a therapeutically effective amount of a formulation comprising a therapeutically effective amount of said peptide as claimed in claim 1, or variant thereof.

13. The method as claimed in claim 12, wherein said formulation is capable of inhibiting one or more of Interferon gamma (IFN-y), thymus and activation regulated chemokine, Interleukin-8, thymic stromal lymphopoietin secretion, IL-β, IL-1β, IL-6, IL-10, IL-12, TNF-α, and CCL2 (MCP1).

14. The method as claimed in claim 12, wherein said administration is in dosage form selected from oral, sub-cutaneous, topical, intra-peritoneal, intra-venous or combination thereof.

15. The method as claimed in claim 12, wherein said therapeutically effective amount of the peptide or variant thereof in said formulation is 0.01 μg/ml to 1000 μg/ml.

16. The method as claimed in claim 12, wherein said administration is at a dosage of about 0.01 mg/kg to 1000 mg/kg.

17. (canceled)

18. The formulation as claimed in claim 4, for treatment, amelioration, alleviation and/or prevention of inflammatory and/or microbial disease and/or one or more symptoms associated thereof.

19. A method of treating a subject infected with inflammatory or microbial diseases for treating, preventing, alleviating and/or ameliorating said inflammatory or microbial diseases or one or more symptoms associated thereof comprising administering a therapeutically effective amount of the peptide as claimed in claim 1, or variant thereof.


SEQUENCE LISTING (11 sequences)

SEQ ID NO: 1 - protein, 7 aa, synthetic construct
  REGION 1..7: note = Synthetic peptide IS 111
FAKKFAK

SEQ ID NO: 2 - DNA, 21 bp, synthetic construct
  misc_feature 1..21: note = Mouse -TNF-alpha Forward primer sequence
  source 1..21: mol_type = other DNA
aagcctgtagcccacgtcgta

SEQ ID NO: 3 - DNA, 24 bp, synthetic construct
  misc_feature 1..24: note = Mouse -TNF-alpha Reverse primer sequence
  source 1..24: mol_type = other DNA
ggcaccactagttggttgtctttg

SEQ ID NO: 4 - DNA, 20 bp, synthetic construct
  misc_feature 1..20: note = Mouse-IL-1 beta Forward primer sequence
  source 1..20: mol_type = other DNA
ccaagcaacgacaaaatacc

SEQ ID NO: 5 - DNA, 22 bp, synthetic construct
  misc_feature 1..22: note = Mouse-IL-1 beta Reverse primer sequence
  source 1..22: mol_type = other DNA
gttgaagacaaaccgtttttcc

SEQ ID NO: 6 - DNA, 21 bp, synthetic construct
  misc_feature 1..21: note = Mouse- IL-6 Forward primer sequence
  source 1..21: mol_type = other DNA
gaacaacgatgatgcacttgc

SEQ ID NO: 7 - DNA, 26 bp, synthetic construct
  misc_feature 1..26: note = Mouse- IL-6 Reverse primer sequence
  source 1..26: mol_type = other DNA
cttcatgtactccaggtagctatggt

SEQ ID NO: 8 - DNA, 22 bp, synthetic construct
  misc_feature 1..22: note = Mouse-CCL2 Forward primer sequence
  source 1..22: mol_type = other DNA
gctacaagaggatcaccagcag

SEQ ID NO: 9 - DNA, 22 bp, synthetic construct
  misc_feature 1..22: note = Mouse-CCL2 Reverse primer sequence
  source 1..22: mol_type = other DNA
gtctggacccattccttcttgg

SEQ ID NO: 10 - DNA, 20 bp, synthetic construct
  misc_feature 1..20: note = GAPDH Forward primer sequence
  source 1..20: mol_type = other DNA
aggccggtgctgagtatgtc

SEQ ID NO: 11 - DNA, 20 bp, synthetic construct
  misc_feature 1..20: note = GAPDH Reverse primer sequence
  source 1..20: mol_type = other DNA
tgcctgcttcaccaccttct